(19) United States
(12) Patent Application Publication
(10) Pub. No.: US 2026/0042840 A1
(43) Pub. Date: Feb. 12, 2026

(54) METHODS OF TREATING TUMOR

(71) Applicants: Bristol-Myers Squibb Company, Princeton, NJ (US); Celgene Corporation, Summit, NJ (US)

(72) Inventors: Ming LEI, Pennington, NJ (US); Xuya WANG, Princeton, NJ (US); Elaine JOSEPH, Summit, NJ (US); Ke XU, Princeton, NJ (US); Danielle Marie GREENAWALT, Princeton, NJ (US); Peter M. SZABO, Princeton, NJ (US); Alice M. WALSH, Princeton, NJ (US); Michael CARLETON, Princeton, NJ (US); Qu ZHANG, Princeton, NJ (US); Jin YAO, Princeton, NJ (US)

(73) Assignees: Bristol-Myers Squibb Company, Princeton, NJ (US); Celgene Corporation, Summit, NJ (US)

(21) Appl. No.: 18/854,920

(22) PCT Filed: Apr. 7, 2023
(86) PCT No.: PCT/US2023/065542

§ 371 Date: Oct. 7, 2024

Related U.S. Application Data
(60) Provisional application No. 63/328,715, filed on Apr. 7, 2022.

(51) Int. Cl. C07K 16/28; A61K 39/00; A61K 39/395; A61P 35/00; C12Q 1/6886

(52) U.S. Cl. CPC C07K 16/2818; A61K 39/39558; A61P 35/00; C12Q 1/6886; A61K 2039/505; A61K 2039/507; A61K 2039/545; C12Q 2600/158

(57) ABSTRACT

The disclosure provides a method for treating a subject afflicted with a tumor comprising administering to the subject a therapeutically effective amount of an anti-PD-1 antibody or antigen-binding portion thereof or an anti-PD-L1 antibody or antigen-binding portion thereof, wherein the subject is identified as having a low stromal gene signature score. In some aspects, the low stromal gene signature score is determined by measuring the expression of a panel of stromal genes in a tumor sample obtained from the subject, wherein the stromal gene panel comprises at least four genes selected from CDH1, CDH2, MMP1, MMP2, ITGA1, ITGA2, ITGA3, ITGA5, ITGA7, ITGA11, TGFB1, and TGFB1; at least four genes selected from TGFB1, TGFBR2, ACTA2, COL4A1, TAGLN, SH3PXD2A, TWIST1, ZEB1, and ZEB2; or MMP2 and MMP9.

### CROSS REFERENCE TO RELATED APPLICATIONS

This PCT application claims the priority benefit of U.S. Provisional Application No. 63/328,715, filed Apr. 7, 2022, which is incorporated by reference herein in its entirety.

### FIELD OF THE DISCLOSURE

The present disclosure provides a method for treating a subject afflicted with a tumor using an immunotherapy.

### BACKGROUND OF THE DISCLOSURE

Human cancers harbor numerous genetic and epigenetic alterations, generating neoantigens potentially recognizable by the immune system (Sjoblom et al., Science (2006) 314(5797):268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibodies such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014). Nivolumab has shown activity in a variety of advanced solid tumors, including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

The immune system and response to immuno-therapy are complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

Some aspects of the present disclosure are directed to a method for treating a human subject afflicted with a tumor comprising (i) identifying a subject exhibiting a low stromal signature score; and (ii) administering to the subject an anti-PD-1 antibody or an anti-PD-L1 antibody; wherein the stromal signature score is determined by measuring the expression of a panel of stroma-related genes (“stromal gene panel”) in a tumor sample obtained from the subject; and wherein the stromal gene panel comprises at least four genes selected from CDH1, CDH2, AMIP1, MMP2, ITGA1, ITGA2, ITGA3, ITGA5, ITGA7, ITGA11, TGFB1, and TGFB1.

Some aspects of the present disclosure are directed to a method for treating a human subject afflicted with a tumor comprising administering an anti-PD-1 antibody or an anti-PD-L1 antibody to the subject, wherein the subject is identified as exhibiting a low stromal signature score prior to the administration; wherein the stromal signature score is determined by measuring the expression of a panel of stroma-related genes (“stromal gene panel”) in a tumor sample obtained from the subject; and wherein the stromal gene panel comprises at least four genes selected from CDH1, CDH2, MMP1, MMP2, ITGA1, ITGA2, ITGA3, ITGA5, ITGA7, ITGA11, TGFB1, and TGFB1.

Some aspects of the present disclosure are directed to a method for identifying a human subject afflicted with a tumor suitable for an anti-PD-1 antibody or an anti-PD-L1 antibody treatment comprising (i) measuring a stromal signature score in a tumor sample obtained from the subject and (ii) administering to the subject an anti-PD-1 antibody or an anti-PD-L1 antibody if the subject exhibits a low stromal signature score; wherein the stromal signature score is determined by measuring the expression of a panel of stroma-related genes (“stromal gene panel”) in the tumor sample obtained from the subject; and wherein the stromal gene panel comprises at least four genes selected from CDH1, CDH2, MMP1, MMP2, ITGA1, ITGA2, ITGA3, ITGA5, ITGA7, ITGA11, TGFB1, and TGFB1.

In some aspects, the stromal gene panel comprises CDH1, CDH2, MMP1, MMP2, ITGA1, ITGA2, ITGA3, ITGA5, ITGA7, ITGA11, TGFB1, and TGFB1 In some aspects, the stromal gene panel consists essentially of (i) CDH1, CDH2, MMP1, MMP2, ITGA1, ITGA2, ITGA3, ITGA5, ITGA7, ITGA11, TGFB1, and TGFB1; and (ii) 1 additional stromal gene, 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, or 15 additional stromal genes. In some aspects, the additional stromal gene is selected from TGFBR2, ACTA2, COL4A1, TAGLN, SH3PXD2A, TWIST1, ZEB1, ZEB2, and MMP9, and any combination thereof. In some aspects, the stromal gene panel consists essentially of CDH1, CDH2, MMP1, MMP2, ITGA1, ITGA2, ITGA3, ITGA5, ITGA7, ITGA11, TGFB1, and TGFB1. In some aspects, the stromal gene panel consists of CDH1, CDH2, MMP1, MMP2, ITGA1, ITGA2, ITGA3, ITGA5, ITGA7, ITGA11, TGFB1, and TGFB1.

Some aspects of the present disclosure are directed to a method for treating a human subject afflicted with a tumor comprising (i) identifying a subject exhibiting a low stromal signature score; and (ii) administering to the subject an anti-PD-1 antibody or an anti-PD-L1 antibody; wherein the stromal signature score is determined by measuring the expression of a panel of stroma-related genes (“stromal gene panel”) in a tumor sample obtained from the subject; and wherein the stromal gene panel comprises at least four genes selected from TGFB1, TGFBR2, ACTA2, COL4A1, TAGLN, SH3PXD2A, TWIST1, ZEB1, and ZEB2.

Some aspects of the present disclosure are directed to a method for treating a human subject afflicted with a tumor comprising administering an anti-PD-1 antibody or an anti-PD-L1 antibody to the subject, wherein the subject is identified as exhibiting a low stromal signature score prior to the administration; wherein the stromal signature score is determined by measuring the expression of a panel of stroma-related genes (“stromal gene panel”) in a tumor sample obtained from the subject; and wherein the stromal gene panel comprises at least four genes selected from TGFB1, TGFBR2, ACTA2, COL4A1, TAGLN, SH3PXD2A, TWIST1, ZEB1, and ZEB2.

Some aspects of the present disclosure are directed to a method for identifying a human subject afflicted with a tumor suitable for an anti-PD-1 antibody or an anti-PD-L1 antibody treatment comprising (i) measuring a stromal signature score in a tumor sample obtained from the subject and (ii) administering to the subject an anti-PD-1 antibody or an anti-PD-L1 antibody if the subject exhibits a low stromal signature score; wherein the stromal signature score is determined by measuring the expression of a panel of stroma-related genes (“stromal gene panel”) in the tumor sample obtained from the subject; and wherein the stromal gene panel comprises at least four genes selected from TGFB1, TGFBR2, ACTA2, COL4A1, TAGLN, SH3PXD2A, TWIST1, ZEB1, and ZEB2. In some aspects, the stromal gene panel comprises TGFB1, TGFBR2, ACTA2, COL4A1, TAGLN, SH3PXD2A, TWIST1, ZEB1, and ZEB2

In some aspects, wherein the stromal gene panel consists essentially of (i) TGFB1, TGFBR2, ACTA2, COL4A1, TAGLN, SH3PXD2A, TWIST1, ZEB1, and ZEB2; and (ii) 1 additional stromal gene, 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, or 15 additional stromal genes. In some aspects, the additional stromal gene is selected from the group consisting of CDH1, CDH2, MMP1, MMP2, ITGA1, ITGA2, ITGA3, ITGA5, ITGA7, ITGA11, TGFB1, and MMP9, and any combination thereof. In some aspects, the stromal gene panel consists essentially of TGFB1, TGFBR2, ACTA2, COL4A1, TAGLN, SH3PXD2A, TWIST1, ZEB1, and ZEB2. In some aspects, the stromal gene panel consists of TGFB1, TGFBR2, ACTA2, COL4A1, TAGLN SH3PXD2A, TWIST1, ZEB1, and ZEB2.

Some aspects of the present disclosure are directed to a method for treating a human subject afflicted with a tumor comprising (i) identifying a subject exhibiting a low stromal signature score; and (ii) administering to the subject an anti-PD-1 antibody or an anti-PD-L1 antibody; wherein the stromal signature score is determined by measuring the expression of a panel of stroma-related genes (“stromal gene panel”) in a tumor sample obtained from the subject; and wherein the stromal gene panel comprises MMP2 and MMP9.

Some aspects of the present disclosure are directed to a method for treating a human subject afflicted with a tumor comprising administering an anti-PD-1 antibody or an anti-PD-L1 antibody to the subject, wherein the subject is identified as exhibiting a low stromal signature score prior to the administration; wherein the stromal signature score is determined by measuring the expression of a panel of stroma-related genes (“stromal gene panel”) in a tumor sample obtained from the subject; and wherein the stromal gene panel comprises MMP2 and MMP9.

Some aspects of the present disclosure are directed to a method for identifying a human subject afflicted with a tumor suitable for an anti-PD-1 antibody or an anti-PD-L1 antibody treatment comprising (i) measuring a stromal signature score in a tumor sample obtained from the subject and (ii) administering to the subject an anti-PD-1 antibody or an anti-PD-L1 antibody if the subject exhibits a low stromal signature score; wherein the stromal signature score is determined by measuring the expression of a panel of stroma-related genes (“stromal gene panel”) in the tumor sample obtained from the subject; and wherein the stromal gene panel comprises MMP2 and MMP9.

In some aspects, the stromal gene panel consists essentially of MMP2 and MMP9, and (ii) 1 additional stromal gene, 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, or 15 additional stromal genes. In some aspects, the additional stromal gene is selected from the group consisting of CDH1, CDH2, MMP1, ITGA1, ITGA2, ITGA3, ITGA5, ITGA7, ITGA11, TGFB1, TGFB1, TGFBR2, ACTA2, COL4A1, TAGLN, SH3PXD2A, TWIST1, ZEB1, and ZEB2, and any combination thereof.

In some aspects, the stromal gene panel consists essentially of MMP2 and MMP9. In some aspects, the stromal gene panel consists of MMP2 and MMP9.

In some aspects, the stromal gene panel consists of less than about 20, less than about 18, less than about 15, less than about 13, less than about 10, less than about 9, less than about 8, less than about 7, less than about 6, or less than about 5 stromal genes.

In some aspects, the low stromal signature score is characterized by a stromal signature score that is lower than an average stromal signature score, wherein the average stromal signature score is determined by averaging or computationally deriving from the stroma signature scores in tumor samples obtained from a population of subjects afflicted with the tumor. In some aspects, the average stromal signature score is determined by averaging or computationally deriving from the stroma signature scores in tumor samples obtained from the population of subjects. In some aspects, the low stromal signature score is characterized by a stromal signature score that is less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, less than about 1% that of the average stromal signature score.

In some aspects, the low stromal signature score is characterized by a stromal signature score that is less than about 75% that of the average stromal signature score. In some aspects, the low stromal signature score is characterized by a stromal signature score that is less than about 50% that of the average stromal signature score.

In some aspects, the tumor sample is a tumor tissue biopsy. In some aspects, the tumor sample is a formalin-fixed, paraffin-embedded tumor tissue or a fresh-frozen tumor tissue.

In some aspects, the expression of the stromal genes in the stromal gene panel is determined by detecting the presence of stromal gene mRNA, the presence of a protein encoded by the stromal gene, or both. In some aspects, the presence of stromal gene mRNA is determined using reverse transcriptase PCR or other PCR technologies, RNA sequencing or other next generation sequencing technologies, or any combination thereof. In some aspects, the presence of the protein encoded by the stromal gene is determined using an IHC assay. In some aspects, the IHC assay is an automated IHC assay.

In some aspects, the anti-PD-1 antibody cross-competes with nivolumab for binding to human PD-1. In some aspects, the anti-PD-1 antibody binds to the same epitope as nivolumab. In some aspects, the anti-PD-1 antibody is a chimeric, humanized or human monoclonal antibody or a portion thereof. In some aspects, the anti-PD-1 antibody comprises a heavy chain constant region which is of a human IgG1 or IgG4 isotype. In some aspects, the anti-PD-1 antibody comprises nivolumab. In some aspects, the anti-PD-1 antibody comprises pembrolizumab.

In some aspects, the anti-PD-1 antibody is administered at a dose ranging from at least about 0.1 mg/kg to at least about 10.0 mg/kg body weight once about every 1, 2 or 3 weeks. In some aspects, the anti-PD-1 antibody is administered at a dose of at least about 3 mg/kg body weight once about every 2 weeks.

In some aspects, the anti-PD-1 antibody or antigen-binding portion thereof is administered at a flat dose. In some aspects, the anti-PD-1 antibody or antigen-binding portion thereof is administered at a flat dose of at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or at least about 550 mg. In some aspects, the anti-PD-1 antibody or antigen-binding portion thereof is administered at a flat dose of about 240 mg. In some aspects, the anti-PD-1 antibody or antigen-binding portion thereof is administered at a flat dose of about 480 mg. In some aspects, the anti-PD-1 antibody or antigen-binding portion thereof is administered at a flat dose about once every 1, 2, 3 or 4 weeks. In some aspects, the anti-PD-1 antibody or antigen-binding portion thereof is administered at a flat dose or about 240 mg once about every two weeks. In some aspects, the anti-PD-1 antibody or antigen-binding portion thereof is administered at a flat dose of about 480 mg once about every four weeks.

In some aspects, the anti-PD-1 antibody is administered for as long as clinical benefit is observed or until unmanageable toxicity or disease progression occurs. In some aspects, the anti-PD-1 antibody is formulated for intravenous administration. In some aspects, the anti-PD-1 antibody is administered at a subtherapeutic dose.

In some aspects, the anti-PD-L1 antibody cross-competes with durvalumab, avelumab, or atezolizumab for binding to human PD-1. In some aspects, the anti-PD-L1 antibody binds to the same epitope as durvalumab, avelumab, or atezolizumab. In some aspects, the anti-PD-L1 antibody comprises durvalumab. In some aspects, the anti-PD-L1 antibody comprises avelumab. In some aspects, the anti-PD-L1 antibody comprises atezolizumab.

In some aspects, the anti-PD-L1 antibody is administered at a dose ranging from 0.1 mg/kg to E20.0 mg/kg body weight once every 2, 3, or 4 weeks. In some aspects, the anti-PD-L1 antibody is administered at a dose of 15 mg/kg body weight once every 3 weeks. In some aspects, the anti-PD-L1 antibody is administered at a dose of 10 mg/kg body weight once every 2 weeks.

In some aspects, the anti-PD-L1 antibody is administered at a flat dose. In some aspects, the anti-PD-L1 antibody is administered at a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg. In some aspects, the anti-PD-L1 antibody is administered as a flat dose about once every 1, 2, 3, or 4 weeks. In some aspects, the anti-PD-L1 antibody is administered as a flat dose of about 1200 mg once every 3 weeks. In some aspects, the anti-PD-L1 antibody is administered as a flat dose of about 800 mg once every 2 weeks.

In some aspects, the method further comprises administering an antibody or an antigen binding fragment thereof that binds specifically to cytotoxic T-lymphocyte-associated protein 4 (CTLA-4) (“an anti-CTLA-4 antibody”). In some aspects, the anti-CTLA-4 antibody cross-competes with ipilimumab or tremelimumab for binding to human CTLA-4. In some aspects, the anti-CTLA-4 antibody binds to the same epitope as ipilimumab or tremelimumab. In some aspects, the anti-CTLA-4 antibody is ipilimumab. In some aspects, the anti-CTLA-4 antibody is tremelimumab. In some aspects, the anti-CTLA-4 antibody is administered at a dose ranging from 0.1 mg/kg to 20.0 mg/kg body weight once every 2, 3, 4, 5, 6, 7, or 8 weeks. In some aspects, the anti-CTLA-4 antibody is administered at a dose of 1 mg/kg body weight once every 6 weeks. In some aspects, the anti-CTLA-4 antibody is administered at a dose of 1 mg/kg body weight once every 4 weeks. In some aspects, the anti-CTLA-4 antibody is administered at a flat dose. In some aspects, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some aspects, the anti-CTLA-4 antibody is administered as a flat dose about once every 2, 3, 4, 5, 6, 7, or 8 weeks.

In some aspects, the tumor is derived from a cancer selected from the group consisting of hepatocellular cancer, gastroesophageal cancer, melanoma, bladder cancer, lung cancer, kidney cancer, head and neck cancer, colon cancer, rectal cancer, and any combination thereof. In some aspects, the tumor is derived from a hepatocellular cancer. In some aspects, the tumor is derived from a gastroesophageal cancer. In some aspects, the tumor is derived from a melanoma. In some aspects, the tumor is relapsed. In some aspects, the tumor is refractory.

In some aspects, the tumor is refractory following at least one prior therapy comprising administration of at least one anticancer agent. In some aspects, the at least one anticancer agent comprises a standard of care therapy. In some aspects, the at least one anticancer agent comprises an immunotherapy.

In some aspects, the tumor is resectable. In some aspects, the tumor is locally advanced. In some aspects, the tumor is metastatic.

In some aspects, the administering treats the tumor. In some aspects, the administering reduces the size of the tumor. In some aspects, the size of the tumor is reduced by at least about 10%, about 20%, about 30%, about 40%, or about 50% compared to the tumor size prior to the administration. In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration. In some aspects, the subject exhibits stable disease after the administration. In some aspects, the subject exhibits a partial response after the administration. In some aspects, the subject exhibits a complete response after the administration.

In some aspects, the method further comprises measuring the TMB status of a biological sample obtained from the subject prior to the administering. In some aspects, the subject is identified as having a TMB status of at least about 10 mutations per megabase examined.

In some aspects, the TMB status is determined by sequencing nucleic acids in the tumor and identifying a genomic alteration in the sequenced nucleic acids. In some aspects, the genomic alteration comprises one or more somatic mutations. In some aspects, the genomic alteration comprises one or more nonsynonymous mutations. In some aspects, the genomic alteration comprises one or more missense mutations.

In some aspects, the genomic alteration comprises one or more alterations selected from the group consisting of a base pair substitution, a base pair insertion, a base pair deletion, a copy number alteration (CNAs), a gene rearrangement, and any combination thereof.

In some aspects, the TMB status of the tumor comprises at least 10 mutations, at least about 11 mutations, at least about 12 mutations, at least about 13 mutations, at least about 14 mutations, at least about 15 mutations, at least about 16 mutations, at least about 17 mutations, at least about 18 mutations, at least about 19 mutations, at least about 20 mutations, at least about 21 mutations, at least about 22 mutations, at least about 23 mutations, at least about 24 mutations, at least about 25 mutations, at least about 26 mutations, at least about 27 mutations, at least about 28 mutations, at least about 29 mutations, or at least about 30 mutations per megabase of genome examined as measured by a FOUNDATIONONE® CDX™ assay.

In some aspects, the TMB status is determined by genome sequencing. In some aspects, the TMB status is determined by exome sequencing. In some aspects, the TMB status is determined by genomic profiling.

In some aspects, the genomic profile comprises at least about 20 genes, at least about 30 genes, at least about 40 genes, at least about 50 genes, at least about 60 genes, at least about 70 genes, at least about 80 genes, at least about 90 genes, at least about 100 genes, at least about 110 genes, at least about 120 genes, at least about 130 genes, at least about 140 genes, at least about 150 genes, at least about 160 genes, at least about 170 genes, at least about 180 genes, at least about 190 genes, at least about 200 genes, at least about 210 genes, at least about 220 genes, at least about 230 genes, at least about 240 genes, at least about 250 genes, at least about 260 genes, at least about 270 genes, at least about 280 genes, at least about 290 genes, at least about 300 genes, at least about 305 genes, at least about 310 genes, at least about 315 genes, at least about 320 genes, at least about 325 genes, at least about 330 genes, at least about 335 genes, at least about 340 genes, at least about 345 genes, at least about 350 genes, at least about 355 genes, at least about 360 genes, at least about 365 genes, at least about 370 genes, at least about 375 genes, at least about 380 genes, at least about 385 genes, at least about 390 genes, at least about 395 genes, or at least about 400 genes. In some aspects, the genomic profile comprises at least about 265 genes. In some aspects, the genomic profile comprises at least about 315 genes. In some aspects, the genomic profile comprises at least about 354 genes.

In some aspects, the genomic profile comprises one or more genes selected from the group consisting of ABL1, BRAF, CHEK1, FANCC, GATA3, JAK2, MITF, PDCD1LG2 (PD-L2), RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf 39), KAT6A (MYST 3), MRE 11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf 30 (EMSY), CTCF, FAT1, GNAQ, KDR, MUTYH, PIK3CG, RUNX1T1, TERT (Promoter only), APC, CARD11, CTNNA1, FBXW7, GNAS, KEAP1, MYC, PIK3R1, SDHA, TET2, AR, CBFB, CTNN B1, FGF10, GPR124, KEL, MYCL (MYC L1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274 (PD-L1), DNMT3A, FGF6, HNF1A, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1 (MEK1), NRAS, PTCH1, SNCAIP, WISP3, AXL, CDK6, EPHB1, FLT1, IGF2, MAP2K2 (MEK2), NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRF11, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof.

In some aspects, the TMB status is measured by a FOUNDATIONONE® CDX™ assay.

In some aspects, the method further comprises identifying a genomic alteration in one or more of ETV4, TMPRSS2, ETV5, BCR, ETV1, ETV6, and MYB. In some aspects, the tumor has a high neoantigen load. In some aspects, the subject has an increased T-cell repertoire.

Some aspects of the present disclosure are directed to a kit for treating a subject afflicted with a tumor, the kit comprising: (a) a dosage ranging from about 4 mg to about 500 mg of an anti-PD-1 antibody; and (b) instructions for using the anti-PD-1 antibody a method disclosed herein. In some aspects, the kit further comprises an anti-CTLA-4 antibody. In some aspects, the kit further comprises an anti-PD-L1 antibody.

Other features and advantages of the instant disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS

FIG. 1 is a schematic representation of the CheckMate 649 study design for exploratory endpoint biomarker assessments of efficacy of NIVO in patients with advanced or metastatic gastric cancer/gastroesophageal junction cancer/esophageal adenocarcinoma in Clinical Trial NCT02872116. CheckMate 649 is a randomized, open-label, global phase 3 study. (ClinicalTrials.gov. NCT02872116). At data cutoff, the minimum follow-up time from concurrent randomization of the last patient to clinical data cutoff was 24.0 months in the NIVO+chemotherapy arm. (Janjigian et al., Lancet 398(10294):27-40 (2021)).

FIG. 2 is a table showing the baseline characteristics of the randomized patients, which were consistent amongst all patient groups.

FIG. 3 is a plot showing the correlation of various biomarkers to each other. CPS and the 4-gene inflammatory GES have moderate correlation. Stroma-related signatures have relatively high correlation with each other. Angiogenesis-related signatures have relatively high correlation with each other. Stroma-related signatures have moderate correlation with angiogenesis-related signatures.

FIG. 4 is a table showing the number of subjects in each biomarker evaluable cohort.

FIG. 5 is a plot showing the association with TMB Score and PD-L1 CPS level. TMB in general was observed to be higher in the CPS>=5 subgroup.

FIGS. 6A-6B are KM curves showing the OS benefit of NIVO+Chemo regardless of TMB status in all randomized subjects, although a trend of higher magnitude of benefit in the TMB>=199 subgroup was observed. 8.3% (57/685) of TMB evaluable subjects were TMB-high (>=199 mutations/exome). In the TMB>=199 mutations/exome subgroup, which only accounted for a very small portion of the population, more than half of the subjects (33/57) were MSI-H.

FIG. 7 is a table showing overall survival benefit by TMB for all TMB-evaluable patients.

FIGS. 8A-8B are KM curves showing the OS benefit of NIVO+Chemo in subjects with a CPS>=5. 9.5% (43/451) of CPS>=5 & TMB evaluable subjects were TMB-high (>=199 mutations/exome). In the CPS>=5 & TMB>=199 mutations/exome subgroup, more than half of the subjects (25/43) were MSI-H.

FIGS. 9A-9B are KM curves showing the OS benefit of NIVO+Chemo in subjects with a CPS<5. 6.1% (14/230) of CPS<5 & TMB evaluable subjects were TMB-high (>=199 mutations/exome). In the CPS<5 & TMB>=199 mutations/exome subgroup, more than half of the subjects (8/14) were MSI-H.

FIG. 10 is a table showing overall survival benefit by TMB for all TMB-evaluable patients with PD-L1 CPS ≥5 and <5.

FIGS. 11A-11B are KM curves showing that the 4-gene inflammatory GES appears to be a prognostic biomarker.

FIGS. 12A-12C are KM curves showing that OS benefit was observed regardless of the 4-gene inflammatory GES in all randomized subjects.

FIGS. 13A-13C are KM curves showing that OS benefit was observed regardless of the 4-gene inflammatory GES in the CPS>=5 subgroup.

FIGS. 14A-14C are KM curves showing OS benefit by the 4-gene inflammatory GES in the CPS<5 subgroup.

FIG. 15 is a table showing overall survival benefit by 4-gene inflammatory GES and PD-L1 CPS for all GES-evaluable patients.

FIGS. 16A-16C are KM curves showing that the 12-EMT signature was found to be associated with better OS benefits.

FIGS. 17A-17C are KM curves showing that the 9-TGF-β signature was found to be associated with better OS benefits.

FIGS. 18A-18C are KM curves showing that the 51-Stroma signature was found to be associated with better OS benefits.

FIGS. 19A-19C are KM curves showing that the 2-MMP signature was found to be associated with better OS benefits.

FIGS. 20A-20C are KM curves showing that the 16-Angio signature was found to be associated with better OS benefits.

FIGS. 21A-21C are KM curves showing that the 6-Angio signature was found to be associated with better OS benefits.

FIG. 22 is a table showing overall survival benefit by stroma-related and angiogenesis GES for all GES-evaluable patients.

FIG. 23 is a table showing overall survival benefit by stroma-related and angiogenesis GES for all GES-evaluable patients with PD-L1 CPS ≥5. PD-L1 CPS was not available/not evaluable/indeterminate for NIVO+chemo, n=1 and chemo, n=3.

FIG. 24 is a table showing overall survival benefit by stroma-related and angiogenesis GES for all GES-evaluable patients with PD-L1 CPS <5. PD-L1 CPS was not available/not evaluable/indeterminate for NIVO+chemo, n=1 and chemo, n=3.

FIGS. 25A-25D are graphical representations of the association between overall survival and TGFb signature for patients administered (i) a combination therapy comprising nivolumab and ipilimumab or (ii) chemotherapy. FIG. 25A provides the median overall survival values (months) for both treatment groups, stratified by 9-TGF-β signature. FIGS. 25B-25D are KM curves showing overall survival observed for each of the treatment groups stratified by high (FIG. 25B), medium (FIG. 25C), and low (FIG. 25D) 9-TGF-β signature.

FIGS. 26A-26D are graphical representations of the association between disease-free survival and TGFb signature for patients administered (i) nivolumab or (ii) placebo. FIG. 26A provides the median disease-free survival values (months) for both treatment groups, stratified by 9-TGF-β signature. FIGS. 26B-26D are KM curves showing disease-free survival observed for each of the treatment groups stratified by high (FIG. 26B), medium (FIG. 26C), and low (FIG. 26D) 9-TGF-β signature.

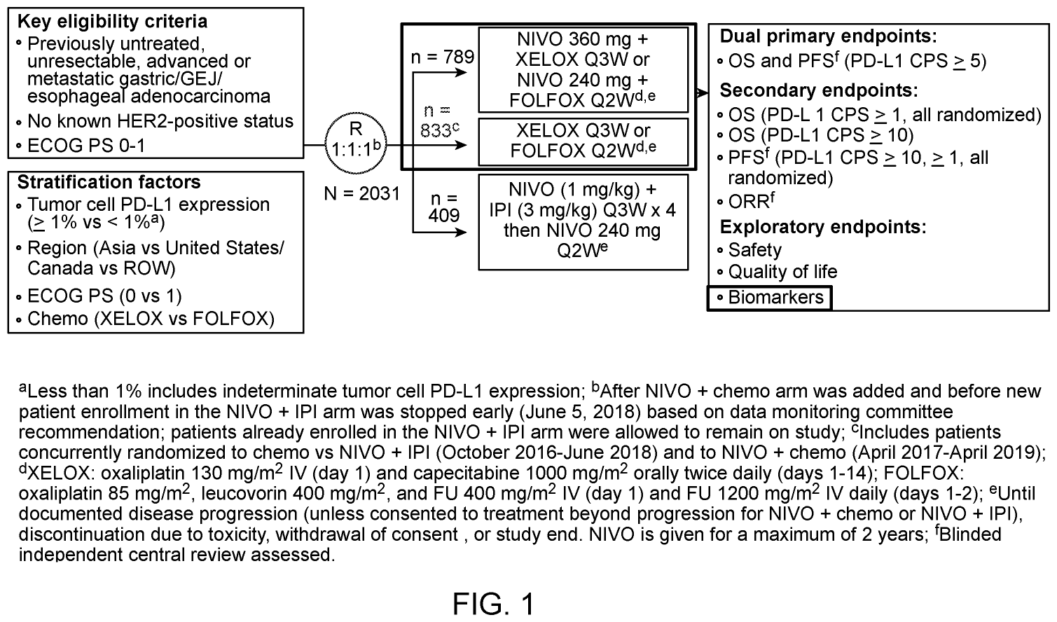

### DETAILED DESCRIPTION OF THE DISCLOSURE

Some aspects of the present disclosure provide a method for treating a human subject afflicted with a tumor comprising (i) identifying a subject displaying a low stromal signature score; and (ii) administering to the subject a PD-1 inhibitor, e.g., an anti-PD-1 antibody or an anti-PD-L1 antibody. The present disclosure also provides a method for treating a human subject afflicted with a tumor comprising administering a PD-1 inhibitor, e.g., an anti-PD-1 antibody or an anti-PD-L1 antibody, wherein the subject is identified as having a low stromal signature score prior to the administration. The present disclosure also provides a method for identifying a human subject afflicted with a tumor suitable for a PD-1 inhibitor, e.g., an anti-PD-1 antibody or an anti-PD-L1 antibody, treatment comprising (i) measuring a stromal signature score in a tumor sample obtained from the subject and (ii) administering to the subject a PD-1 inhibitor, e.g., an anti-PD-1 antibody or an anti-PD-L1 antibody if the subject has a low stromal signature score.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the immunotherapy, e.g., the anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, the term “anti-PD-1 antibody” includes a full antibody having two heavy chains and two light chains that specifically binds to PD-1 and antigen-binding portions of the full antibody. Non limiting examples of the antigen-binding portions are shown elsewhere herein.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1, an anti-PD-L1 antibody binds specifically to PD-L1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody or an anti-PD-L1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD-1 antibody).

The use of the term “fixed dose” with regard to a method of the disclosure means that two or more different antibodies in a single composition (e.g., anti-PD-1 antibody and anti-CTLA-4 antibody or an anti-PD-L1 antibody and an anti-CTLA-4 antibody) are present in the composition in particular (fixed) ratios with each other. In some aspects, the fixed dose is based on the weight (e.g., mg) of the antibodies. In certain aspects, the fixed dose is based on the concentration (e.g., mg/ml) of the antibodies. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody or an anti-PD-L1 antibody) to mg second antibody (e.g., anti-CTLA-4 antibody). For example, the 3:1 ratio of an anti-PD-1 antibody and an anti-CTLA-4 antibody can mean that a vial can contain about 240 mg of the anti-PD-1 antibody and 80 mg of the anti-CTLA-4 antibody or about 3 mg/ml of the anti-PD-1 antibody and 1 mg/ml of the anti-CTLA-4 antibody.

The term “weight-based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred aspects of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using an assay described herein. Alternatively, this property of a composition can be evaluated by examining the ability of the compound to inhibit cell growth, such inhibition can be measured in vitro by assays known to the skilled practitioner. In some aspects described herein, tumor regression can be observed and continue for a period of at least about 20 days, at least about 40 days, or at least about 60 days.

The term “tumor mutation burden” (TMB) as used herein refers to the number of somatic mutations in a tumor's genome and/or the number of somatic mutations per area of the tumor's genome. Germline (inherited) variants are excluded when determining TMB, because the immune system has a higher likelihood of recognizing these as self Tumor mutation burden (TMB) can also be used interchangeably with “tumor mutation load,” “tumor mutational burden,” or “tumor mutational load.”

TMB is a genetic analysis of a tumor's genome and, thus, can be measured by applying sequencing methods well known to those of skill in the art. The tumor DNA can be compared with DNA from patient-matched normal tissue to eliminate germline mutations or polymorphisms.

In some aspects, TMB is determined by sequencing tumor DNA using a high-throughput sequence technique, e.g., next-generation sequencing (NGS) or an NGS-based method. In some aspects, the NGS-based method is selected from whole genome sequencing (WGS), whole exome sequencing (WES), or comprehensive genomic profiling (CGP) of cancer gene panels such as FOUNDATIONONE CDX™ and MSK-IMPACT clinical tests. In some aspects, TMB, as used herein, refers to the number of somatic mutations per megabase (Mb) of DNA sequenced. In one aspect, TMB is measured using the total number of nonsynonymous mutations, e.g., missense mutation (i.e. changing a particular amino acid in the protein) and/or nonsense (causing premature termination and thus truncation of the protein sequence), identified by normalizing matched tumor with germline samples to exclude any inherited germline genetic alterations. In another aspect, TMB is measured using the total number of missense mutations in a tumor. In order to measure TMB, a sufficient amount of sample is required. In one aspect, tissue sample (for example, a minimum of 10 slides) is used for evaluation. In some aspects, TMB is expressed as NsMs per megabase (NsM/Mb). 1 megabase represents 1 million bases.

The TMB status can be a numerical value or a relative value, e.g., high, medium, or low; within the highest fractile, or within the top tertile, of a reference set.

The term “high TMB” as used herein refers to a number of somatic mutations in a tumor's genome that is above a number of somatic mutations that is normal or average. In some aspects, a TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500; in other aspects a high TMB has a score of at least at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250; and, in a particular aspect, a high TMB has a score of at least 243.

In other aspects, a “high TMB” refers to a TMB within the highest fractile of the reference TMB value. For example, all subject's with evaluable TMB data are grouped according to fractile distribution of TMB, i.e., subjects are rank ordered from highest to lowest number of genetic alterations and divided into a defined number of groups. In one aspect, all subjects with evaluable TMB data are ranked ordered and divided into thirds, and a “high TMB” is within the top tertile of the reference TMB value. In a particular aspect, the tertile boundaries are 0<100 genetic alterations; 100 to 243 genetic alterations; and >243 genetic alterations. It should be understood that, once rank ordered, subjects with evaluable TMB data can be divided into any number of groups, e.g., quartiles, quintiles, etc.

In some aspects, a “high TMB” refers to a TMB of at least about 20 mutations/tumor, at least about 25 mutations/tumor, at least about 30 mutations/tumor, at least about 35 mutations/tumor, at least about 40 mutations/tumor, at least about 45 mutations/tumor, at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, or at least about 100 mutations/tumor. In some aspects, a “high TMB” refers to a TMB of at least about 105 mutations/tumor, at least about 110 mutations/tumor, at least about 115 mutations/tumor, at least about 120 mutations/tumor, at least about 125 mutations/tumor, at least about 130 mutations/tumor, at least about 135 mutations/tumor, at least about 140 mutations/tumor, at least about 145 mutations/tumor, at least about 150 mutations/tumor, at least about 175 mutations/tumor, or at least about 200 mutations/tumor. In certain aspects, a tumor having a high TMB has at least about 100 mutations/tumor.

The “high TMB” can also be referred to as the number of mutations per megabase of tumor genome sequenced, e.g., as measured by a mutation assay, e.g., FOUNDATIONONE® CDX™ assay. In one aspect, the high TMB refers to at least about 9, at least about 10, at least about 11, at least 12, at least about 13, at least about 14, at least about 15, at least about 16, at least about 17, at least about 18, at least about 19, or at least about 20 mutations per megabase of genome as measured by a FOUNDATIONONE® CDX™ assay. In a particular aspect, the “high TMB” refers to at least 10 mutations per megabase of genome sequenced by a FOUNDATIONONE® CDX™ assay.

As used herein, the term “medium TMB” refers to a number of somatic mutations in a tumor's genome that is at or around a number of somatic mutations that is normal or average and the term “low TMB” refers to a number of somatic mutations in a tumor's genome that is below a number of somatic mutations that is normal or average. In a particular aspect, a “high TMB” has a score of at least 243, a “medium TMB” has a score of between 100 and 242, and a “low TMB” has a score of less than 100 (or between 0 and 100). The “medium or low TMB” refers to less than 9 mutations per megabase of genome sequenced, e.g., as measured by a FOUNDATIONONE® CDX™ assay.

The term “reference TMB value” as referred to herein can be the TMB value shown in Table 9.

In some aspects, TMB status can correlate with smoking status. In particular, subjects who currently or formerly smoke(d) often have more genetic alterations, e.g., missense mutations, than subjects who never smoke(d).

A tumor with a high TMB can also have a high neoantigen load. As used herein, the term “neoantigen” refers to a newly formed antigen that has not been previously recognized by the immune system. A neoantigen can be a protein or peptide that is recognized as foreign (or non-self) by the immune system. Transcription of a gene in the tumor genome harboring a somatic mutation results in mutated mRNA that, when translated, gives rise to a mutated protein, which is then processed and transported to the ER lumen and binds to MHC class I complex, facilitating T-cell recognition of the neoantigen. Neoantigen recognition can promote T-cell activation, clonal expansion, and differentiation into effector and memory T-cells. Neoantigen load can correlate with TMB. In some aspects, TMB is assessed as a surrogate for measuring tumor neoantigen load. The TMB status of a tumor, can be used as a factor, alone or in combination with other factors, in determining whether a patient is likely to benefit from a particular anti-cancer agent or type of treatment or therapy, e.g., a combination therapy comprising (a) an anti-PD-1 antibody or an anti-PD-L1 antibody and (b) an anti-CTLA-4 antibody. In one aspect, a high TMB status (or a high TMB) indicates an enhanced likelihood of benefit from immuno-oncology and, thus, can be used to identify patients more likely to benefit from therapy of a combination therapy comprising (a) an anti-PD-1 antibody or an anti-PD-L1 antibody and (b) an anti-CTLA-4 antibody. Similarly, tumors with high tumor neoantigen load and high TMB are more likely to be immunogenic than tumors with low neoantigen load and low TMB. In addition, high-neoantigen/high-TMB tumors are more likely to be recognized as non-self by the immune system, thus triggering an immune-mediated antitumor response. In one aspect, a high TMB status and a high neoantigen load indicate an enhanced likelihood of benefit from immuno-oncology, e.g., a combination therapy comprising (a) an anti-PD-1 antibody or an anti-PD-L1 antibody and (b) an anti-CTLA-4 antibody. As used herein, the term “benefit from therapy” refers to an improvement in one or more of overall survival, progression-free survival, partial response, complete response, and overall response rate and can also include a reduction in tumor growth or size, a decrease in severity of disease symptoms, an increase in frequency and duration of disease symptom-free periods, or a prevention of impairment or disability due to the disease affliction.

Other factors, e.g., environmental factors, can associate with TMB status. For example, smoking status of patients with NSCLC was correlated with TMB distribution, whereby current and former smokers had higher median TMB compared with those patients who had never smoked. See Peters et al., AACR, Apr. 1-5, 2017, Washington, D.C. The presence of a driver mutation in NSCLC tumors was associated with younger age, female sex, and non-smoker status. See Singal et al., ASCO, Jun. 1-5, 2017; Chicago, IL. A trend associating the presence of driver mutations, such as EGFR, ALK, or KRAS, with lower TMB was observed (P=0.06). Davis et al., AACR, Apr. 1-5, 2017, Washington, D.C.

The term “somatic mutation” as used herein refers to an acquired alteration in DNA that occurs after conception. Somatic mutations can occur in any of the cells of the body except the germ cells (sperm and egg) and therefore are not passed on to children. These alterations can, but do not always, cause cancer or other diseases. The term “germline mutation” refers to a gene change in a body's reproductive cell (egg or sperm) that becomes incorporated into the DNA of every cell in the body of the offspring. Germline mutations are passed on from parents to offspring. Also called a “hereditary mutation.” In the analysis of TMB, germline mutations are considered as a “baseline,” and are subtracted from the number of mutations found in the tumor biopsy to determine the TMB within the tumor. As germline mutations are found in every cell in the body, their presence can be determined via less invasive sample collections than tumor biopsies, such as blood or saliva. Germline mutations can increase the risk of developing certain cancers, and can play a role in the response to chemotherapy.

The term “measuring” or “measured” or “measurement” when referring to TMB status means determining a measurable quantity of somatic mutations in a biological sample of the subject. It will be appreciated that measuring can be performed by sequencing nucleic acids, e.g., cDNA, mRNA, exoRNA, ctDNA, and cfDNA, in the sample. The measuring is performed on a subject's sample and/or a reference sample or samples and can, for example, be detected de novo or correspond to a previous determination. The measuring can be performed, for example, using PCR methods, qPCR methods, Sanger sequencing methods, genomic profiling methods (including comprehensive gene panels), exome sequencing methods, genome sequencing methods, and/or any other method disclosed herein, as is known to a person of skill in the art. In some aspects, the measuring identifies a genomic alteration in the sequenced nucleic acids. The genomic (or gene) profiling methods can involve panels of a predetermined set of genes, e.g., 150-500 genes, and in some instances the genomic alterations evaluated in the panel of genes are correlated with total somatic mutations evaluated. As used herein when referring to sequencing, the term “gene” includes DNA coding regions (e.g., exons), DNA non-coding regions associated with a coding region (e.g., introns and promoters), and mRNA transcripts.

The term “genomic alteration” as used herein refers to a change (or mutation) in the nucleotide sequence of the genome of a tumor, which change is not present in the germline nucleotide sequence, and which in some aspects is a nonsynonymous mutation including, but not limited to, a base pair substitution, a base pair insertion, a base pair deletion, a copy number alteration (CNA), a gene rearrangement, and any combination thereof. In a particular aspect, the genomic alterations measured in the biological sample are missense mutations.

The term “whole genome sequencing” or “WGS,” as used herein, refers to a method of sequencing the entire genome. The term “whole exome sequencing” or “WES,” as used herein, refers to a method of sequencing all the protein-coding regions (exons) of the genome.

A “cancer gene panel,” “hereditary cancer panel,” “comprehensive cancer panel,” or “multigene cancer panel,” as used herein, refers to a method of sequencing a subset of targeted cancer genes, including coding regions, introns, promoters, and/or mRNA transcripts. In some aspects, the CGP comprises sequencing at least about 15, at least about 20, at least about 25, at least about 30, at least about 35, at least about 40, at least about 45, or at least about 50 targeted cancer genes.

The term “genomic profiling assay,” “comprehensive genomic profiling,” or “CGP” refers to an assay that analyzes a panel of genes and select introns for in vitro diagnosis. CGP is a combination of NGS and targeted bioinformatics analysis to screen for mutations in known clinically relevant cancer genes. This method can be used to catch mutations that are missed by testing “hotspots” (e.g., BRCA1/BRCA2 mutations or microsatellite markers). In some aspects, the CGP further includes one or more mRNA transcript, non-coding RNA, and/or promoter region. In one aspect, the genes in the panel are cancer-related genes. In another aspect, a genomic profiling assay is a FOUNDATIONONE© assay.

The term “harmonization” refers to a study conducted to determine the comparability between two or more measures and/or diagnostic tests. Harmonization studies provide a systematic approach to address questions of how diagnostic tests compare with each other, as well as their interchangeability when used to determine the biomarker status of a patient's tumor. In general, at least one well-characterized measure and/or diagnostic test is used as a standard for comparison with others. Concordance assessment is often utilized in harmonization studies.

The term “concordance,” as used herein, refers to a degree of agreement between two measurements and/or diagnostic tests. Concordance can be established using both qualitative and quantitative methods. Quantitative methods to assess concordance differ based on the type of measurement. A particular measurement can be expressed either as 1) a categorical/dichotomized variable or 2) a continuous variable. A “categorical/dichotomized variable” (e.g., above or below TMB cut-off) may use percent agreements, such as overall percent agreement (OPA), positive percent agreement (PPA), or negative percent agreement (NPA), to assess concordance. A “continuous variable” (e.g., TMB by WES) uses Spearman's rank correlation or Pearson's correlation coefficient (r), which takes on values −1≤r≤+1, to assess concordance across a spectrum of values (Note r=+1 or −1 means that each of the variables is perfectly correlated). The term “analytical concordance” refers to the degree of agreement in the performance (e.g., identification of biomarkers, genomic alteration types, and genomic signatures, and assessment of test reproducibility) of two assays or diagnostic tests to support clinical use. The term “clinical concordance” refers to the degree of agreement in how the two assays or diagnostic tests correlate with clinical outcome.

The term “microsatellite instability” or “MSI” refers to a change that occurs in the DNA of certain cells (such as tumor cells) in which the number of repeats of microsatellites (short, repeated sequences of DNA) is different than the number of repeats that was in the DNA when it was inherited. MSI can be high microsatellite instability (MSI-H) or low microsatellite instability (MSI-L). Microsatellites are short tandem DNA repeat sequences of 1-6 bases. These are prone to DNA replication errors, which are repaired by mismatch repair (MMR). Hence microsatellites are good indicators of genome instability, especially deficient mismatch repair (dMMR). MSI is usually diagnosed by screening 5 microsatellite markers (BAT-25, BAT-26, NR21, NR24, and NR27). MSI-H represents the presence of at least 2 unstable markers among 5 microsatellite markers analyzed (or ≥30% of the markers if a larger panel is used). MSI-L means instability of 1 MSI marker (or 10%-30% of markers in larger panels). MSS means the absence of an unstable microsatellite marker.

The term “biological sample” as used herein refers to biological material isolated from a subject. The biological sample can contain any biological material suitable for determining target gene expression or TMB, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one aspect, the sample is a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As used herein, the terms “2-MMP” and “2-gene matrix metallopeptidase” are used interchangeably and refer to a gene panel comprising MMP2 and MMP9. See Table 16.

As used herein, the terms “6-Angio” and “6-gene angiogenesis” are used interchangeably and refer to a gene panel comprising VEGFA, KDR, ESM1, PECAM1, ANGPTL4, and CD34. See Table 16. As used herein, the terms “16-Angio” and “16-gene angiogenesis” are used interchangeably and refer to a gene panel comprising VEGFA, CD34, ANGPTL4, KDR, TEK, NDUFA4L2, ANGPT2, ESM1, CXCR7, SEMA5B, FLT1, TIE1, CDH6, DLL4, FLT4, and ENPEP. See Table 16.

As used herein, the terms “12-EMT” and “12-gene epithelial-to-mesenchymal transition” are used interchangeably and refer to a gene panel comprising CDH1, CDH2, MIP1, MMP2, ITGA1, ITGA2, ITGA3, ITGA5, ITGA7, ITGA11, TGFB1, and TGFB1. See Table 16.

As used herein, the terms “9-TGF-β” and “9-gene transforming growth factor beta” are used interchangeably and refer to a gene panel comprising TGFB1, TGFBR2, ACTA2, COL4A1, TAGLN, SH3PXD2A, TWIST1, ZEB1, and ZEB2. See Table 16.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Abbreviations used herein are defined throughout the present disclosure. A list of additional abbreviations is provided in Table 1.

TABLE 1
List of Abbreviations

| Term | Definition |
|---|---|
| 2-MMP | 2-gene matrix metallopeptidase |
| 6-Angio | 6-gene angiogenesis |
| 9-TGF-β | 9-gene transforming growth factor beta |
| 12-EMT | 12-gene epithelial-to-mesenchymal transition |
| 16-Angio | 16-gene angiogenesis |
| 51-Stroma | 51-gene stroma/epithelial-to-mesenchymal transition/ |
| | transforming growth factor beta |
| 1L | first line |
| ALK | anaplastic lymphoma kinase |
| AUC | area under the concentration-time curve |
| BSA | body surface area |
| cfDNA | cell-free DNA |
| chemo | chemotherapy |
| CI | confidence interval |
| CPS | combined positive score |
| CR | complete response |
| ctDNA | circulating tumor DNA |
| EAC | esophageal adenocarcinoma |
| ECOG | Eastern Cooperative Oncology Group |
| ECOG PS | Eastern Oncology Cooperative Group performance status |
| EGFR | epidermal growth factor receptor |
| ELISA | enzyme-linked immunosorbent assay |
| exoRNA | exosomal RNA |
| FOLFOX | oxaliplatin + leucovorin + FU |
| FU | fluorouracil |
| GC | gastric cancer |
| GEJ | gastroesophageal junction |
| GEJC | gastroesophageal junction cancer |
| GES | gene expression signature |
| HER2 | human epidermal growth factor receptor 2 |
| HR | hazard ratio |
| IPI | ipilimumab |
| IV | intravenous |
| MSI | microsatellite instability |
| MSI-H | microsatellite instability high |
| MSS | microsatellite stable |
| N | number of subjects or observations |
| NCCN | National Comprehensive Cancer Network |
| NIVO | nivolumab |
| NSCLC | non-small cell lung cancer |
| ORR | overall response rate |
| OS | overall survival |
| PD-1 | programmed death-1 |
| PD-L1 | programmed death ligand 1 |
| PFS | progression-free survival |
| Q2W | every 2 weeks |
| Q3W | every 3 weeks |
| R | randomization |
| RECIST | response evaluation criteria in solid tumors |
| ROW | rest of world |
| TMB | tumor mutational burden |
| XELOX | oxaliplatin + capecitabine |

Various aspects of the disclosure are described in further detail in the following subsections.

### II. Methods of the Disclosure

Some aspects of the present disclosure are directed to methods of treating a human subject afflicted with a tumor comprising (i) identifying a subject exhibiting a low stromal signature score; and (ii) administering to the subject an anti-PD-1 antibody or an anti-PD-L1 antibody. Some aspects of the present disclosure are directed to methods of treating a human subject afflicted with a tumor comprising administering an anti-PD-1 antibody or an anti-PD-L1 antibody to the subject, wherein the subject is identified as exhibiting a low stromal signature score prior to the administration. Some aspects of the present disclosure are directed to methods of treating a tumor in a human subject, comprising administering to the subject a PD-1 inhibitor, e.g., an anti-PD-1 antibody or anti-PD-L1 antibody, wherein the tumor exhibits a low stromal signature score prior to the administration. Some aspects of the present disclosure are directed to methods for identifying a human subject afflicted with a tumor suitable for an anti-PD-1 antibody or an anti-PD-L1 antibody treatment comprising (i) measuring a stromal signature score in a tumor sample obtained from the subject and (ii) administering to the subject an anti-PD-1 antibody or an anti-PD-L1 antibody if the subject exhibits a low stromal signature score.

In some aspects, the stromal signature score is determined by measuring the expression of a panel of stroma-related genes (“stromal gene panel”) in a tumor sample obtained from the subject.

In some aspects, the stromal gene panel comprises at least one, at least two, at least three, at least four, at least five, at least six, at least seven, at least eight, at least ten, at least eleven, or at least twelve genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least two genes selected from CDH1, CDH2, MIP1, MMP2, ITGA1, ITGA2, ITGA3, ITGA5, ITGA7, ITGA11, TGFB1, and TGFB1. In some aspects, the stromal gene panel comprises at least three genes selected from CDH1, CDH2, MIP1, MMP2, ITGA1, ITGA2, ITGA3, ITGA5, ITGA7, ITGA11, TGFB1, and TGFB1. In some aspects, the stromal gene panel comprises at least four genes selected from CDH1, CDH2, MIP1, MIP2, ITGA1, ITGA2, ITGA3, ITGA5, ITGA7, ITGA11, TGFB1, and TGFB1. In some aspects, the stromal gene panel comprises at least five genes selected from CDH1, CDH2, MMP1, MIP2, ITGA1, ITGA2, ITGA3, ITGA5, ITGA7, ITGA11, TGFB1, and TGFB1. In some aspects, the stromal gene panel comprises at least six genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least seven genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least eight genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least nine genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least ten genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least eleven genes selected from CDH1, CDH2, MMP1, MMP2, ITGA1, ITGA2, ITGA3, ITGA5, ITGA7, ITGA11, TGFB1, and TGFB1. In some aspects, the stromal gene panel comprises at least twelve genes selected from CDH1, CDH2, MMP1, MMP2, ITGA1, ITGA2, ITGA3, ITGA5, ITGA7, ITGA11, TGFB1, and TGFB1.

In some aspects, the stromal gene panel comprises at least one, at least two, at least three, at least four, at least five, at least six, at least seven, at least eight genes selected from TGFB1, TGFBR2, ACTA2, COL4A1, TAGLN, SH3PXD2A, TWIST1, ZEB1, and ZEB2. In some aspects, the stromal gene panel comprises at least two genes selected from TGFB1, TGFBR2, ACTA2, COL4A1, TAGLN, SH3PXD2A, TWIST1, ZEB1, and ZEB2. In some aspects, the stromal gene panel comprises at least three genes selected from TGFB1, TGFBR2, ACTA2, COL4A1, TAGLN, SH3PXD2A, TWIST1, ZEB1, and ZEB2. In some aspects, the stromal gene panel comprises at least four genes selected from TGFB1, TGFBR2, ACTA2, COL4A1, TAGLN, SH3PXD2A, TWIST1, ZEB1, and ZEB2. In some aspects, the stromal gene panel comprises at least five genes selected from TGFB1, TGFBR2, ACTA2, COL4A1, TAGLN, SH3PXD2A, TWIST1, ZEB1, and ZEB2. In some aspects, the stromal gene panel comprises at least six genes selected from TGFB, TGFBR2, ACTA2, COL4A1, TAGLN, SH3PXD2A, TWIST1, ZEB1, and ZEB2. In some aspects, the stromal gene panel comprises at least seven genes selected from TGFB1, TGFBR2, ACTA2, COL4A1, TAGLN, SH3PXD2A, TWIST1, ZEB1, and ZEB2. In some aspects, the stromal gene panel comprises at least eight genes selected from TGFB1, TGFBR2, ACTA2, COL4A1, TAGLN, SH3PXD2A, TWIST1, ZEB1, and ZEB2.

In some aspects, the stromal gene panel comprises MMP2 and MMP9.

In some aspects, the stromal gene panel consists of less about 20, less than about 19, less than about 18, less than about 17, less than about 16, less than about 15, less than about 14, less than about 13, less than about 12, less than about 11, less than about 10, less than about 9, less than about, less than about 8, less than about 7, less than about 6, or less than about 5 stromal genes. In some aspects, the stromal gene panel consists of less than 20 genes. In some aspects, the stromal gene panel consists of less than 19 genes. In some aspects, the stromal gene panel consists of less than 18 genes. In some aspects, the stromal gene panel consists of less than 17 genes. In some aspects, the stromal gene panel consists of less than 16 genes. In some aspects, the stromal gene panel consists of less than 15 genes. In some aspects, the stromal gene panel consists of less than 14 genes. In some aspects, the stromal gene panel consists of less than 13 genes. In some aspects, the stromal gene panel consists of less than 12 genes. In some aspects, the stromal gene panel consists of less than 11 genes. In some aspects, the stromal gene panel consists of less than 10 genes. In some aspects, the stromal gene panel consists of less than 9 genes. In some aspects, the stromal gene panel consists of less than 8 genes. In some aspects, the stromal gene panel consists of less than 7 genes. In some aspects, the stromal gene panel consists of less than 6 genes. In some aspects, the stromal gene panel consists of less than 5 genes. In certain aspects, the stromal gene panel consists of 4 genes.

In some aspects, the stromal gene panel consists essentially of CDH1, CDH2, MMP1, MIP2, ITGA1, ITGA2, ITGA3, ITGA5, ITGA7, ITGA11, TGFB1, and TGFB1. In some aspects, the stromal gene panel consists essentially of TGFB1, TGFBR2, ACTA2, COL4A1, TAGLN, SH3PXD2A, TWIST1, ZEB1, and ZEB2. In some aspects, the stromal gene panel consists essentially of MMP2 and MMP9.

In some aspects, the stromal gene panel consists of CDH1, CDH2, AMIP1, MIP2, ITGA1, ITGA2, ITGA3, ITGA5, ITGA7, ITGA11, TGFB1, and TGFB1. In some aspects, the stromal gene panel consists of TGFB1, TGFBR2, ACTA2, COL4A1, TAGLN, SH3PXD2A, TWIST1, ZEB1, and ZEB2. In some aspects, the stromal gene panel consists of MMP2 and MMP9.

In some aspects, the stromal gene panel consists essentially of (or consists of) (i) CDH1, CDH2, MMP1, MMP2, ITGA1, ITGA2, ITGA3, ITGA5, ITGA7, ITGA11, TGFB1, and TGFB1, and (ii) 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, 15 additional stromal genes, 16 additional stromal genes, or 17 additional stromal genes. In some aspects, the stromal gene panel consists essentially of (or consists of) (i) TGFB1, TGFBR2, ACTA2, COL4A1, TAGLN, SH3PXD2A, TWIST1, ZEB1, and ZEB2, and (ii) 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, 15 additional stromal genes, 16 additional stromal genes, or 17 additional stromal genes. In some aspects, the stromal gene panel consists essentially of (or consists of) (i) MMP2 and MMP9, and (ii) 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, 15 additional stromal genes, 16 additional stromal genes, or 17 additional stromal genes.

Various genes associated with stroma are known in the art and can be included in the stromal gene panel disclosed herein. For example, the additional stromal gene can be selected from the group consisting of CDH1, CDH2, MMP1, MMP2, ITGA1, ITGA2, ITGA3, ITGA5, ITGA7, ITGA11, TGFB1, TGFB1, TGFBR2, ACTA2, COL4A1, TAGLN, SH3PXD2A, TWIST1, ZEB1, ZEB2, and MMP9, and any combination thereof

### I.A. Stromal Signature Score

The stromal signature score, as used herein, is a measurement of the combined expression level of the genes present in the stromal gene panel, e.g., comprising, consisting essentially of, or consisting of (i) CDH1, CDH2, MMP1, MMP2, ITGA1, ITGA2, ITGA3, ITGA5, ITGA7, ITGA11, TGFB1, and TGFB1; (ii) TGFB1, TGFBR2, ACTA2, COL4A1, TAGLN, SH3PXD2A, TWIST1, ZEB1, and ZEB2; or (iii) MMP2 and MMP9, in a sample obtained from the subject. Any biological sample comprising one or more tumor cell can be used in the methods disclosed herein. In some aspects, the sample is selected from a tumor biopsy, a blood sample, a serum sample, or any combination thereof. In certain aspects, the sample is a tumor biopsy collected from the subject prior to administration of the anti-PD-1 antibody or the anti-PD-L1 antibody. In particular aspects, the sample obtained from the subject is a formalin-fixed tumor biopsy. In some aspects, the sample obtained from the subject is a paraffin-embedded tumor biopsy. In some aspects, the sample obtained from the subject is a fresh-frozen tumor biopsy.

Any method known in the art for measuring the expression of a particular gene or a panel of genes can be used in the methods of the present disclosure. In some aspects, the expression of one or more of the stromal genes in the stromal gene panel is determined by detecting the presence of mRNA transcribed from the stromal gene, the presence of a protein encoded by the stromal gene, or both.

In some aspects, the expression of one or more of the stromal genes is determined by measuring the level of stromal gene mRNA, e.g., by measuring the level of one or more of CDH1 mRNA, CDH2 mRNA, MMP1 mRNA, MMP2 mRNA, ITGA1 mRNA, ITGA2 mRNA, ITGA3 mRNA, ITGA5 mRNA, ITGA7 mRNA, ITGA1 1 mRNA, TGFB1 mRNA, TGFB1 mRNA, TGFBR2 mRNA, ACTA2 mRNA, COL4A1 mRNA, TAGLN mRNA, SH3PXD2A mRNA, TWIST1 mRNA, ZEB1 mRNA, ZEB2 mRNA, and MMP9 mRNA, in a sample obtained from the subject. In certain aspects, the stromal gene score is determined by measuring the level of CDH1 mRNA, CDH2 mRNA, MMP1 mRNA, MMP2 mRNA, ITGA1 mRNA, ITGA2 mRNA, ITGA3 mRNA, ITGA5 mRNA, ITGA7 mRNA, ITGA11 mRNA, TGFB1 mRNA, and TGFB1 mRNA; or TGFB1 mRNA, TGFBR2 mRNA, ACTA2 mRNA, COL4A1 mRNA, TAGLN mRNA, SH3PXD2A mRNA, TWIST1 mRNA, ZEB1 mRNA, and ZEB2 mRNA; or MMP2 mRNA and MMP9 mRNA in a sample obtained from the subject. Any method known in the art can be used to measure the level of the stromal gene mRNA. In some aspects, the stromal gene mRNA is measured using a PCR technology. In some aspects, the stromal gene mRNA is measured using reverse transcriptase PCR. In some aspects, the stromal gene mRNA is measured using RNA in situ hybridization. In some aspects, the stromal gene mRNA is measured using RNA sequencing. In some aspects, the stromal gene mRNA is measured using a next generation sequencing technology. In some aspects, the stromal gene mRNA is measured using RNA-fluorescence in situ hybridization. In some aspects, the stromal gene mRNA is measured using RNA in situ hybridization. In some aspects, the stromal gene mRNA is measured using nanostring. In some aspects, the stromal gene mRNA is measured using HTG EdgeSeq. In some aspects, the stromal gene mRNA is measured using a microarray.

In some aspects, the expression of one or more of the stromal genes is determined by measuring the level of stromal gene protein, e.g., by measuring the level of one or more of CDH1, CDH2, MMP1, MMP2, ITGA1, ITGA2, ITGA3, ITGA5, ITGA7, ITGA11, TGFB1, TGFB1, TGFBR2, ACTA2, COL4A1, TAGLN, SH3PXD2A, TWIST1, ZEB1, ZEB2, and MMP9, in a sample obtained from the subject. In certain aspects, the stromal gene score is determined by measuring the level of CDH1, CDH2, MMP1, MMP2, ITGA1, ITGA2, ITGA3, ITGA5, ITGA7, ITGA11, TGFB1, and TGFB1; or TGFB1, TGFBR2, ACTA2, COL4A1, TAGLN, SH3PXD2A, TWIST1, ZEB1, and ZEB2; or MMP2 and MMP9 in a sample obtained from the subject. Any method known in the art can be used to measure the level of the stromal gene protein. In some aspects, the stromal gene protein is measured using an immunohistochemistry (IHC) assay. In certain aspects, the IHC is an automated IHC.

In some aspects, the expression of one or more of the stromal genes of the stromal gene panel is normalized relative to the expression of one or more housekeeping genes. In some aspects, the one or more housekeeping genes are made up of genes that have relatively consistent expression across various tumor types in various subjects.

In some aspects, raw gene expression values are normalized following standard gene expression profiling (GEP) protocols. In these aspects, gene expression signature scores can be calculated as the median or average of the log 2-transformed normalized and scaled expression values across all of the target genes in the signature, and presented on a linear scale. In certain aspects, scores have positive or negative values, depending on whether gene expression is up- or down-regulated under a particular condition.

In certain aspects, a low stromal signature score is characterized by a stromal signature score that is lower than a reference stromal signature score. In some aspects, the reference stromal signature score is an average stromal signature score. In some aspects, the average stromal signature score is determined by computationally deriving from the stroma signature scores in tumor samples obtained from a population of subjects afflicted with the tumor. In some aspects, the average stromal signature score is determined by measuring the expression of the genes present in the stromal gene panel in tumor samples obtained from a population of subjects, and calculating the average for the population of subjects. In some aspects, each member of the population of subjects is afflicted with the same tumor type as the subject being administered the anti-PD-1 antibody, the anti-PD-L1 antibody, the anti-CTLA-4 antibody, or any combination thereof.

In some aspects, a low stromal score is characterized by a stromal signature score that is less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, less than about 1% that of the average stromal signature score. In certain aspects, a low stromal score is characterized by a stromal signature score that is less than about 95% that of an average stromal signature score. In certain aspects, a low stromal score is characterized by a stromal signature score that is less than about 90% that of an average stromal signature score. In certain aspects, a low stromal score is characterized by a stromal signature score that is less than about 85% that of an average stromal signature score. In certain aspects, a low stromal score is characterized by a stromal signature score that is less than about 80% that of an average stromal signature score. In certain aspects, a low stromal score is characterized by a stromal signature score that is less than about 75% that of an average stromal signature score. In certain aspects, a low stromal score is characterized by a stromal signature score that is less than about 70% that of an average stromal signature score. In certain aspects, a low stromal score is characterized by a stromal signature score that is less than about 65% that of an average stromal signature score. In certain aspects, a low stromal score is characterized by a stromal signature score that is less than about 60% that of an average stromal signature score. In certain aspects, a low stromal score is characterized by a stromal signature score that is less than about 55% that of an average stromal signature score. In certain aspects, a low stromal score is characterized by a stromal signature score that is less than about 50% that of an average stromal signature score. In certain aspects, a low stromal score is characterized by a stromal signature score that is less than about 45% that of an average stromal signature score. In certain aspects, a low stromal score is characterized by a stromal signature score that is less than about 40% that of an average stromal signature score. In certain aspects, a low stromal score is characterized by a stromal signature score that is less than about 35% that of an average stromal signature score. In certain aspects, a low stromal score is characterized by a stromal signature score that is less than about 30% that of an average stromal signature score. In certain aspects, a low stromal score is characterized by a stromal signature score that is less than about 25% that of an average stromal signature score. In certain aspects, a low stromal score is characterized by a stromal signature score that is less than about 20% that of an average stromal signature score. In certain aspects, a low stromal score is characterized by a stromal signature score that is less than about 15% that of an average stromal signature score. In certain aspects, a low stromal score is characterized by a stromal signature score that is less than about 10% that of an average stromal signature score. In certain aspects, a low stromal score is characterized by a stromal signature score that is less than about 9% that of an average stromal signature score. In certain aspects, a low stromal is characterized by a stromal signature score that is less than about 8% that of an average stromal signature score. In certain aspects, a low stromal score is characterized by a stromal signature score that is less than about 7% that of an average stromal signature score. In certain aspects, a low stromal score is characterized by a stromal signature score that is less than about 6% that of an average stromal signature score. In certain aspects, a low stromal score is characterized by a stromal signature score that is less than about 5% that of an average stromal signature score.

In some aspects, a low stromal score is characterized by a stromal signature score that is at least about 1.25-fold, at least about 1.30-fold, at least about 1.35-fold, at least about 1.40-fold, at least about 1.45-fold, at least about 1.50-fold, at least about 1.55-fold, at least about 1.60-fold, at least about 1.65-fold, at least about 1.70-fold, at least about 1.75-fold, at least about 1.80-fold, at least about 1.85-fold, at least about 1.90-fold, at least about 1.95-fold, at least about 2-fold, at least about 2.25-fold, at least about 2.50-fold, at least about 2.75-fold, at least about 3-fold, at least about 3.25-fold, at least about 3.50-fold, at least about 3.75-fold, or at least about 400-fold lower than an average stromal signature score. In certain aspects, a low stromal score is characterized by a stromal signature score that is at least about 1.25-fold lower than an average stromal signature score. In certain aspects, a low stromal score is characterized by a stromal signature score that is at least about 1.30-fold lower than an average stromal signature score. In certain aspects, a low stromal score is characterized by a stromal signature score that is at least about 1.35-fold lower than an average stromal signature score. In certain aspects, a low stromal score is characterized by a stromal signature score that is at least about 1.40-fold lower than an average stromal signature score. In certain aspects, a low stromal score is characterized by a stromal signature score that is at least about 1.45-fold lower than an average stromal signature score. In certain aspects, a low stromal score is characterized by a stromal signature score that is at least about 1.50-fold lower than an average stromal signature score. In certain aspects, a low stromal score is characterized by a stromal signature score that is at least about 1.55-fold lower than an average stromal signature score. In certain aspects, a low stromal score is characterized by a stromal signature score that is at least about 1.60-fold lower than an average stromal signature score. In certain aspects, a low stromal score is characterized by a stromal signature score that is at least about 1.65-fold lower than an average stromal signature score. In certain aspects, a low stromal score is characterized by a stromal signature score that is at least about 1.70-fold lower than an average stromal signature score. In certain aspects, a low stromal score is characterized by a stromal signature score that is at least about 1.75-fold lower than an average stromal signature score. In certain aspects, a low stromal score is characterized by a stromal signature score that is at least about 1.80-fold lower than an average stromal signature score. In certain aspects, a low stromal score is characterized by a stromal signature score that is at least about 1.85-fold lower than an average stromal signature score. In certain aspects, a low stromal score is characterized by a stromal signature score that is at least about 1.90-fold lower than an average stromal signature score. In certain aspects, a low stromal score is characterized by a stromal signature score that is at least about 1.95-fold lower than an average stromal signature score. In certain aspects, a low stromal score is characterized by a stromal signature score that is at least about 2-fold lower than an average stromal signature score. In certain aspects, a low stromal score is characterized by a stromal signature score that is at least about 2.25-fold lower than an average stromal signature score. In certain aspects, a low stromal score is characterized by a stromal signature score that is at least about 2.50-fold lower than an average stromal signature score. In certain aspects, a low stromal score is characterized by a stromal signature score that is at least about 2.75-fold lower than an average stromal signature score. In certain aspects, a low stromal score is characterized by a stromal signature score that is at least about 3-fold lower than an average stromal signature score. In certain aspects, a low stromal score is characterized by a stromal signature score that is at least about 3.25-fold lower than an average stromal signature score. In certain aspects, a low stromal score is characterized by a stromal signature score that is at least about 3.50-fold lower than an average stromal signature score. In certain aspects, a low stromal score is characterized by a stromal signature score that is at least about 3.75-fold lower than an average stromal signature score. In certain aspects, a low stromal score is characterized by a stromal signature score that is at least about 4-fold lower than an average stromal signature score.

In certain aspects, a low stromal signature score is characterized by a stromal signature score of at least about 0.5, wherein the stromal signature score is determined according to a method disclosed herein. In some aspects, a low stromal signature score is characterized by a stromal signature score of at least about 0.75, wherein the stromal signature score is determined according to a method disclosed herein. In some aspects, a low stromal signature score is characterized by a stromal signature score of at least about 1.0, wherein the stromal signature score is determined according to a method disclosed herein. In some aspects, a low stromal signature score is characterized by a stromal signature score of at least about 1.25, wherein the stromal signature score is determined according to a method disclosed herein. In some aspects, a low stromal signature score is characterized by a stromal signature score of at least about 1.50, wherein the stromal signature score is determined according to a method disclosed herein. In some aspects, a low stromal signature score is characterized by a stromal signature score of at least about 1.75, wherein the stromal signature score is determined according to a method disclosed herein. In some aspects, a low stromal signature score is characterized by a stromal signature score of at least about 2.0, wherein the stromal signature score is determined according to a method disclosed herein. In some aspects, a low stromal signature score is characterized by a stromal signature score of at least about 2.25, wherein the stromal signature score is determined according to a method disclosed herein. In some aspects, a low stromal signature score is characterized by a stromal signature score of at least about 2.5, wherein the stromal signature score is determined according to a method disclosed herein. In some aspects, a low stromal signature score is characterized by a stromal signature score of at least about 2.75, wherein the stromal signature score is determined according to a method disclosed herein. In some aspects, a low stromal signature score is characterized by a stromal signature score of at least about 3.0, wherein the stromal signature score is determined according to a method disclosed herein.

### II.B. Tumor Mutation Burden (TMB)

Certain aspects of the present disclosure are directed to a method for treating a human subject afflicted with a tumor comprising administering a PD-1 inhibitory, e.g., an anti-PD-1 antibody or an anti-PD-L1 antibody, to the subject, wherein the subject is identified as exhibiting (i) a low stromal signature score and (ii) a tumor mutation burden (TMB) status of at least about 10 mutations per megabase of genes examined prior to the administration. The disclosure is based on the fact that tumor immunogenicity is directly related to TMB and/or neoantigen load.

As a tumor grows, it accumulates somatic mutations not present in germline DNA. TMB refers to the number of somatic mutations in a tumor's genome and/or the number of somatic mutations per area of the tumor genome (after taking into account germline variant DNA). The acquisition of somatic mutations and, thus, a higher TMB can be influenced by distinct mechanisms, such as exogenous mutagen exposure (e.g., tobacco smoking) and DNA mismatch repair mutations (e.g., MSI in colorectal and esophageal cancers). In solid tumors, about 95% of mutations are single-base substitutions. (Vogelstein et al., Science (2013) 339:1546-1558.) A “nonsynonymous mutation” herein refers to a nucleotide mutation that alters the amino acid sequence of a protein. Missense mutations and nonsense mutations can be both nonsynonymous mutations. A “missense mutation” herein refers to a nonsynonymous point mutation in which a single nucleotide change results in a codon that codes for a different amino acid. A “nonsense mutation” herein refers to a nonsynonymous point mutation in which a codon is changed to a premature stop codon that leads to truncation of the resulting protein.

In some aspects, somatic mutations can be expressed at the RNA and/or protein level, resulting in neoantigens (also referred to as neoepitopes). Neoantigens can influence an immune-mediated anti-tumor response. For example, neoantigen recognition can promote T-cell activation, clonal expansion, and differentiation into effector and memory T-cells.

As a tumor develops, early clonal mutations (or “trunk mutations”) can be carried by most or all tumor cells, while late mutations (or “branch mutations”) can occur in only a subset of tumor cells or regions. (Yap et al., Sci Tranl Med (2012) 4:1-5; Jamai-Hanjani et al., (2015) Clin Cancer Res 21:1258-1266.) As a result, neoantigens derived from clonal “trunk” mutations are more widespread in the tumor genome than “branch” mutations and, thus, can lead to a high number of T cells reactive against the clonal neoantigen. (McGranahan et al., (2016) 351:1463-1469.) Generally, tumors with a high TMB can also have a high neoantigen load, which can lead to high tumor immunogenicity and increased T-cell reactivity and anti-tumor response. As such, cancers with a high TMB can respond well to treatment with immunotherapies, e.g., an anti-PD-1 antibody or anti-PD-L1 antibody.

Advances in sequencing technologies allow for evaluation of the tumor's genomic mutation landscape. Any sequencing methods known to those of skill in the art can be used to sequence nucleic acids from the tumor genome (e.g., obtained from a biological sample from a subject afflicted with a tumor). In one aspect, PCR or qPCR methods, Sanger sequencing methods, or next-generation sequencing (“NGS”) methods (such as genomic profiling, exome sequencing, or genome sequencing) can be used to measure TMB. In some aspects, the TMB status is measured using genomic profiling. Genomic profiling involves analyzing nucleic acids from tumor samples, including coding and non-coding regions, and can be performed using methods having integrated optimized nucleic acid selection, read alignment, and mutation calling. In some aspects, gene profiling provides next generation sequencing (NGS)-based analysis of tumors that can be optimized on a cancer-by-cancer, gene-by-gene, and/or site-by-site basis. Genome profiling can integrate the use of multiple, individually tuned, alignment methods or algorithms to optimize performance in sequencing methods, particularly in methods that rely on massively parallel sequencing of a large number of diverse genetic events in a large number of diverse genes. Genomic profiling provides for a comprehensive analysis of a subject's cancer genome, with clinical grade quality, and the output of the genetic analysis can be contextualized with relevant scientific and medical knowledge to increase the quality and efficiency of cancer therapy.

### II.B.1. Genomic Profiling

Genomic profiling involves a panel of a predefined set of genes comprising as few as five genes or as many as 1000 genes, about 25 genes to about 750 genes, about 100 genes to about 800 genes, about 150 genes to about 500 genes, about 200 genes to about 400 genes, about 250 genes to about 350 genes. In one aspect, the genomic profile comprises at least 300 genes, at least 305 genes, at least 310 genes, at least 315 genes, at least 320 genes, at least 325 genes, at least 330 genes, at least 335 genes, at least 340 genes, at least 345 genes, at least 350 genes, at least 355 genes, at least 360 genes, at least 365 genes, at least 370 genes, at least 375 genes, at least 380 genes, at least 385 genes, at least 390 genes, at least 395 genes, or at least 400 genes. In another aspect, the genomic profile comprises at least 325 genes. In a particular aspect, the genomic profile comprises at least 315 cancer-related genes and introns in 28 genes (FOUNDATIONONE®) or the complete DNA coding sequence of 406 genes, introns in 31 genes with rearrangements, and the RNA sequence (cDNA) of 265 genes (FOUNDATIONONE® Heme). In another aspect, the genomic profile comprises 26 genes and 1000 associated mutations (EXODX® Solid Tumor). In yet another aspect, the genomic profile comprises 76 genes (Guardant360). In yet another aspect, the genomic profile comprises 73 genes (Guardant360). In another aspect, the genomic profile comprises 354 genes and introns in 28 genes for rearrangements (FOUNDATIONONE® CDX™). In certain aspects, the genomic profile is FOUNDATIONONE® F1CDx. In another aspect, the genomic profile comprises 468 genes (MSKR-IMPACT™). One or more genes can be added to the genome profile as more genes are identified to be related to oncology.

II.B.1.a. FOUNDATIONONE® Assay

The FOUNDATIONONE® assay is comprehensive genomic profiling assay for solid tumors, including but not limited to solid tumors of the lung, colon, and breast, melanoma, and ovarian cancer. The FOUNDATIONONE® assay uses a hybrid-capture, next-generation sequencing test to identify genomic alterations (base substitutions, insertions and deletions, copy number alterations, and rearrangements) and select genomic signatures (e.g., TMB and microsatellite instability). The assay covers 322 unique genes, including the entire coding region of 315 cancer-related genes, and selected introns from 28 genes. The full list of FOUNDATIONONE® assay genes is provided in Tables 2 and 3. See FOUNDATIONONE: Technical Specifications, Foundation Medicine, Inc., available at FoundationMedicine.com, last visited Mar. 16, 2018, which is incorporated by reference herein in its entirety.

TABLE 2
List of genes wherein entire coding sequences are assayed in the FOUNDATIONONE ® assay.

| | | | | | | | | | |
|---|---|---|---|---|---|---|---|---|---|
| ABL1 | BRAF | CHEK1 | FANCC | GATA3 | JAK2 | MITF | PDCD1LG2 | RBM10 | STAT4 |
| | | | | | | | (PD-L2) | | |
| ABL2 | BRCA1 | CHEK2 | FANCD2 | GATA4 | JAK3 | MLH1 | PDGFRA | RET | STK11 |
| ACVR1B | BRCA2 | CIC | FANCE | GATA6 | JUN | MPL | PDGFRB | RICTOR | SUFU |
| AKT1 | BRD4 | CREBBP | FANCF | GID4 | KAT6A | MRE11A | PDK1 | RNF43 | SYK |
| | | | | (C17orf39) | (MYST3) | | | | |
| AKT2 | BRIP1 | CRKL | FANCG | GLl1 | KDM5A | MSH2 | PIK3C2B | ROS1 | TAF1 |
| AKT3 | BTG1 | CRLF2 | FANCL | GNA11 | KDM5C | MSH6 | PIK3CA | RPTOR | TBX3 |
| ALK | BTK | CSF1R | FAS | GNA13 | KDM6A | MTOR | PIK3CB | RUNX1 | TERC |
| AMER1 | C11orf30 | CTCF | FAT1 | GNAQ | KDR | MUTYH | PIK3CG | RUNX1T1 | TERT |
| (FAM123B) | (EMSY) | | | | | | | | (Promoter |
| | | | | | | | | | only) |
| APC | CARD11 | CTNNA1 | FBXW7 | GNAS | KEAP1 | MYC | PIK3R1 | SDHA | TET2 |
| AR | CBFB | CTNNB1 | FGF10 | GPR124 | KEL | MYCL | PIK3R2 | SDHB | TGFBR2 |
| | | | | | | (MYCL1) | | | |
| ARAF | CBL | CUL3 | FGF14 | GRIN2A | KIT | MYCN | PLCG2 | SDHC | TNFAIP3 |
| ARFRP1 | CCND1 | CYLD | FGF19 | GRM3 | KLHL6 | MYD88 | PMS2 | SDHD | TNFRSF14 |
| ARID1A | CCND2 | DAXX | FGF23 | GSK3B | KMT2A | NF1 | POLD1 | SETD2 | TOP1 |
| | | | | | (MLL) | | | | |
| ARID1B | CCND3 | DDR2 | FGF3 | H3F3A | KMT2C | NF2 | POLE | SF3B1 | TOP2A |
| | | | | | (MLL3) | | | | |
| ARID2 | CCNE1 | DICER1 | FGF4 | HGF | KMT2D | NFE2L2 | PPP2R1A | SLIT2 | TP53 |
| | | | | | (MLL2) | | | | |
| ASXL1 | CD274 | DNMT3A | FGF6 | HNF1A | KRAS | NFKBIA | PRDM1 | SMAD2 | TSC1 |
| | (PD-L1) | | | | | | | | |
| ATM | CD79A | DOT1L | FGFR1 | HRAS | LMO1 | NKX2-1 | PREX2 | SMAD3 | TSC2 |
| ATR | CD79B | EGFR | FGFR2 | HSD3B1 | LRP1B | NOTCH1 | PRKAR1A | SMAD4 | TSHR |
| ATRX | CDC73 | EP300 | FGFR3 | HSP90AA1 | LYN | NOTCH2 | PRKCI | SMARCA4 | U2AF1 |
| AURKA | CDH1 | EPHA3 | FGFR4 | IDH1 | LZTR1 | NOTCH3 | PRKDC | SMARCB1 | VEGFA |
| AURKB | CDK12 | EPHA5 | FH | IDH2 | MAGI2 | NPM1 | PRSS8 | SMO | VHL |
| AXIN1 | CDK4 | EPHA7 | FLCN | IGF1R | MAP2K1 | NRAS | PTCH1 | SNCAIP | WISP3 |
| | | | | | (MEK1) | | | | |
| AXL | CDK6 | EPHB1 | FLT1 | IGF2 | MAP2K2 | NSD1 | PTEN | SOCS1 | WT1 |
| | | | | | (MEK2) | | | | |
| BAP1 | CDK8 | ERBB2 | FLT3 | IKBKE | MAP2K4 | NTRK1 | PTPN11 | SOX10 | XPO1 |
| BARD1 | CDKN1A | ERBB3 | FLT4 | IKZF1 | MAP3K1 | NTRK2 | QKI | SOX2 | ZBTB2 |
| BCL2 | CDKN1B | ERBB4 | FOXL2 | IL7R | MCL1 | NTRK3 | RAC1 | SOX9 | ZNF217 |
| BCL2L1 | CDKN2A | ERG | FOXP1 | INHBA | MDM2 | NUP93 | RAD50 | SPEN | ZNF703 |
| BCL2L2 | CDKN2B | ERRFl1 | FRS2 | INPP4B | MDM4 | PAK3 | RAD51 | SPOP | |
| BCL6 | CDKN2C | ESR1 | FUBP1 | IRF2 | MED12 | PALB2 | RAF1 | SPTA1 | |
| BCOR | CEBPA | EZH2 | GABRA6 | IRF4 | MEF2B | PARK2 | RANBP2 | SRC | |
| BCORL1 | CHD2 | FAM46C | GATA1 | IRS2 | MEN1 | PAX5 | RARA | STAG2 | |
| BLM | CHD4 | FANCA | GATA2 | JAK1 | MET | PBRM1 | RB1 | STAT3 | |

TABLE 3
List of genes wherein selected introns are assayed
in the FOUNDATIONONE ® assay.

| | | | | | | |
|---|---|---|---|---|---|---|
| ALK | BRCA1 | ETV1 | FGFR1 | MSH2 | NTRK1 | RARA |
| BCL2 | BRCA2 | ETV4 | FGFR2 | MYB | NTRK2 | RET |
| BCR | BRD4 | ETV5 | FGFR3 | MYC | PDGFRA | ROS1 |
| BRAF | EGFR | ETV6 | KIT | NOTCH2 | RAF1 | TMPRSS2 |

II.B.1.b. EXODX® Solid Tumor Assay

In one aspect, TMB is measured using the EXODX® Solid Tumor assay. The EXODX® Solid Tumor assay is an exoRNA- and cfDNA-based assay, which detects actionable mutations in cancer pathways. The EXODX® Solid Tumor assay is a plasma-based assay that does not require a tissue sample. The EXODX® Solid Tumor assay covers 26 genes and 1000 mutations. The specific genes covered by the EXODX® Solid Tumor assay are shown in Table 4. See Plasma-Based Solid Tumor Mutation Panel Liquid Biopsy, Exosome Diagnostics, Inc., available at exosomedx.com, last accessed on Mar. 25, 2019.

TABLE 4
Genes covered by the EXODX ® Solid Tumor assay.

| | | | | | | | | |
|---|---|---|---|---|---|---|---|---|
| BRAF | MEK1 | KIT | ROS1 | ALK | PTEN | TP53 | FGFR3 | TSC2 |
| NRAS | KRAS | PDGFRA | RET | AKT1 | DH2 | NOTCH1 | NTRK1 | CDKN2A |
| PIK3CA | EGFR | EML4-ALK | HER-2/ | ARv7 | mTOR | Hedgehog | TSC1 | |
| | | | NEU; ERBB2 | | | | | |

II.B.1.c. Guardant360 Assay

In some aspects, TMB status is determined using the Guardant360 assay. The Guardant360 assay measures mutations in at least 73 genes (Table 5), 23 indels (Table 6), 18 CNVs (Table 7), and 6 fusion genes (Table 8). See GuardantHealth.com, last accessed on Mar. 25, 2019.

TABLE 5
Guardant360 assay genes.

| | | | | | | |
|---|---|---|---|---|---|---|
| AKT1 | CCND2 | EZH2 | IDH1 | MLH1 | PDGFRA | SMAD4 |
| ALK | CCNE1 | FBXW7 | IDH2 | MPL | PIK3CA | SMO |
| APC | CDH1 | FGFR1 | JAK2 | MTOR | PTEN | STK11 |
| AR | CDK4 | FGFR2 | JAK3 | MYC | PTPN11 | TERT |
| | | | | | | (including promoter) |
| ARAF | CDK6 | FGFR3 | KIT | NF1 | RAF1 | TP53 |
| ARID1A | CDKN2A | GATA3 | KRAS | NFE2L2 | RB1 | TSC1 |
| ATM | CTNNB1 | GNA11 | MAP2K1 | NOTCH1 | RET | VHL |
| BRAF | DDR2 | GNAQ | MAP2K2 | NPM1 | RHEB | |
| BRCA1 | EGFR | GNAS | MAPK1 | NRAS | RHOA | |
| BRCA2 | ERBB2 | HNF1A | MAPK3 | NTRK1 | RIT1 | |
| CCND1 | ESR1 | HRAS | MET | NTRK3 | ROS1 | |

TABLE 6
Guardant360 assay indels.

| | | | | | | | |
|---|---|---|---|---|---|---|---|
| APC | BRCA1 | CDKN2A | GATA3 | MLH1 | PDGFRA | SMAD4 | TSC1 |
| ARID1A | BRCA2 | EGFR | KIT | MTOR | PTEN | STK11 | VHL |
| ATM | CDH1 | ERBB2 | MET | NF1 | RB1 | TP53 | |

TABLE 7
Guardant360 assay amplifications (CNVs).

AR
BRAF
CCND1
CCND2
CCNE1
CDK4
CDK6
EGFR
ERBB2
FGFR1
FGFR2
KIT
KRAS
MET
MYC
PDGFRA
PIK3CA
RAF1

TABLE 8
Guardant360 assay fusions.

ALK
FGFR2
FGFR3
NTRK1
RET
ROS1

II.B.1.d. ILLUMINA® TruSight Assay

In some aspects, TMB is determined using the TruSight Tumor 170 assay (ILLUMINA). The TruSight Tumor 170 assay is a next-generation sequencing assay that covers 170 genes associated with common solid tumors, which simultaneously analyzes DNA and RNA. The TruSight Tumor 170 assay assesses fusions, splice variants, insertions/deletions, single nucleotide variants (SNVs), and amplifications. The TruSight Tumor 170 assay gene lists are shown in Tables 12-14.

TABLE 9
TruSight Tumor 170 assay genes (amplifications).

AKT2
ALK
AR
ATM
BRAF
BRCA1
BRCA2
CCND1
CCND3
CCNE1
CDK4
CDK6
CHEK1
CHEK2
EGFR
ERBB2
ERBB3
ERCC1
ERCC2
ESR1
FGF1
FGF10
FGF14
FGF19
FGF2
FGF23
FGF3
FGF4
FGF5
FGF6
FGF7
FGF8
FGF9
FGFR1
FGFR2
FGFR3
FGFR4
JAK2
KIT
KRAS
LAMP1
MDM2
MDM4
MET
MYC
MYCL1
MYCN
NRAS
NRG1
PDGFRA
PDGFRB
PIK3CA
PIK3CB
PTEN
RAF1
RET
RICTOR
RPS6KB1
TFRC

TABLE 10
TruSight Tumor 170 assay genes (fusions).

| | | | | | | | |
|---|---|---|---|---|---|---|---|
| ABL1 | BRCA1 | ERG | FGFR1 | JAK2 | MSH2 | NTRK2 | PPARG |
| AKT3 | BRCA2 | ESR1 | FGFR2 | KDR | MYC | NTRK3 | RAF1 |
| ALK | CDK4 | ETS1 | FGFR3 | KIF5B | NOTCH1 | PAX3 | RET |
| AR | CSF1R | ETV1 | FGFR4 | KIT | NOTCH2 | PAX7 | ROS1 |
| AXL | EGFR | ETV4 | FLI1 | KMT2A | NOTCH3 | PDGFRA | RPS6KB1 |
| | | | | (MLL) | | | |
| BCL2 | EML4 | ETV5 | FLT1 | MET | NRG1 | PDGFRB | TMPRSS2 |
| BRAF | ERBB2 | EWSR1 | FLT3 | MLLT3 | NTRK1 | PIK3CA | |

TABLE 11
TruSight Tumor 170 assay genes (small variants).

| | | | | | | | | | |
|---|---|---|---|---|---|---|---|---|---|
| AKT1 | BRCA2 | CHEK1 | ESR1 | FGF7 | HRAS | MET | NF1 | PMS2 | SLX4 |
| AKT2 | BRIP1 | CHEK2 | EZH2 | FGF8 | IDH1 | MLH1 | NOTCH1 | PPP2R2A | SMAD4 |
| AKT3 | BTK | CREBBP | FAM175A | FGF9 | IDH2 | MLLT3 | NOTCH2 | PTCH1 | SMARCB1 |
| ALK | CARD11 | CSF1R | FANCI | FGFR1 | INPP4B | MPL | NOTCH3 | PTEN | SMO |
| APC | CCND1 | CTNNB1 | FANCL | FGFR2 | JAK2 | MRE11A | NPM1 | PTPN11 | SRC |
| AR | CCND2 | DDR2 | FBXW7 | FGFR3 | JAK3 | MSH2 | NRAS | RAD51 | STK11 |
| ARID1A | CCNE1 | DNMT3A | FGF1 | FGFR4 | KDR | MSH3 | NRG1 | RAD51B | TERT |
| ATM | CD79A | EGFR | FGF10 | FLT1 | KIT | MSH6 | PALB2 | RAD51C | TET2 |
| ATR | CD79B | EP300 | FGF14 | FLT3 | KMT2A | MTOR | PDGFRA | RAD51D | TP53 |
| | | | | | (MLL) | | | | |
| BAP1 | CDH1 | ERBB2 | FGF2 | FOXL2 | KRAS | MUTYH | PDGFRB | RAD54L | TSC1 |
| BARD1 | CDK12 | ERBB3 | FGF23 | GEN1 | MAP2K1 | MYC | PIK3CA | RB1 | TSC2 |
| BCL2 | CDK4 | ERBB4 | FGF3 | GNA11 | MAP2K2 | MYCL1 | PIK3CB | RET | VHL |
| BCL6 | CDK6 | ERCC1 | FGF4 | GNAQ | MCL1 | MYCN | PIK3CD | RICTOR | XRCC2 |
| BRAF | CDKN2A | ERCC2 | FGF5 | GNAS | MDM2 | MYD88 | PIK3CG | ROS1 | |
| BRCA1 | CEBPA | ERG | FGF6 | HNF1A | MDM4 | NBN | PIK3R1 | RPS6KB1 | |

ILB.1.e. FOUNDATIONONE® FICDx Assay

FOUNDATIONONE® CDX™ (“F1CDx”) is a next generation sequencing based in vitro diagnostic device for detection of substitutions, insertion and deletion alterations (indels), and copy number alterations (CNAs) in 324 genes and select gene rearrangements, as well as genomic signatures including microsatellite instability (MSI) and tumor mutation burden (TIVB) using DNA isolated from formalin-fixed paraffin embedded (FFPE) tumor tissue specimens. F1CDx is approved by the United States Food and Drug Administration (FDA) for several tumor indications, including NSCLC, melanoma, breast cancer, colorectal cancer, and ovarian cancer.

The F1CDx assay employs a single DNA extraction method from routine FFPE biopsy or surgical resection specimens, 50-1000 ng of which will undergo whole-genome shotgun library construction and hybridization-based capture of all coding exons from 309 cancer-related genes, one promoter region, one non-coding (ncRNA), and selected intronic regions from 34 commonly rearranged genes, 21 of which also include the coding exons. Tables 12 and 13 provide the complete list of genes included in F1CDx. In total, the assay detects alterations in a total of 324 genes. Using the ILLUMIN4A® HiSeq 4000 platform, hybrid capture-selected libraries are sequenced to high uniform depth (targeting >500× median coverage with >9900 of exons at coverage >100×). Sequence data is then processed using a customized analysis pipeline designed to detect all classes of genomic alterations, including base substitutions, indels, copy number alterations (amplifications and homozygous gene deletions), and selected genomic rearrangements (e.g., gene fusions). Additionally, genomic signatures including microsatellite instability (MSI) and tumor mutation burden (TMB) are reported.

TABLE 12
Genes with full coding exonic regions included in FOUNDATIONONE ® CDX ™
for the detection of substitutions, insertions and deletions (indels), and copy number alterations (CNAs).

| | | | | | | | | | |
|---|---|---|---|---|---|---|---|---|---|
| ABL1 | BRCA2 | CDKN2C | ERCC4 | GATA3 | KDM5C | MRE11A | PARP2 | RAD51 | SOX9 |
| ACVR1B | BRD4 | CEBPA | ERG | GATA4 | KDM6A | MSH2 | PARP3 | RAD51B | SPEN |
| AKT1 | BRIP1 | CHEK1 | ERRFI1 | GATA6 | KDR | MSH3 | PAX5 | RAD51C | SPOP |
| AKT2 | BTG1 | CHEK2 | ESR1 | GID4 | KEAP1 | MSH6 | PBRM1 | RAD51D | SRC |
| | | | | (C17orf39) | | | | | |
| AKT3 | BTG2 | CIC | EZH2 | GNA11 | KEL | MST1R | PDCD1 | RAD52 | STAG2 |
| ALK | BTK | CREBBP | FAM46C | GNA13 | KIT | MTAP | PDCD1LG2 | RAD54L | STAT3 |
| ALOX12B | C11orf30 | CRKL | FANCA | GNAQ | KLHL6 | MTOR | PDGFRA | RAF1 | STK11 |
| AMER1 | CALR | CSF1R | FANCC | GNAS | KMT2A | MUTYH | PDGFRB | RARA | SUFU |
| | | | | | (MLL) | | | | |
| APC | CARD11 | CSF3R | FANCG | GRM3 | KMT2D | MYC | PDK1 | RB1 | SYK |
| | | | | | (MLL2) | | | | |
| AR | CASP8 | CTCF | FANCL | GSK3B | KRAS | MYCL | PIK3C2B | RBM10 | TBX3 |
| ARAF | CBFB | CTNNA1 | FAS | H3F3A | LTK | MYCN | PIK3C2G | REL | TEK |
| ARFRP1 | CBL | CTNNB1 | FBXW7 | HDAC1 | LYN | MYD88 | PIK3CA | RET | TET2 |
| ARLD1A | CCND1 | CUL3 | FGF10 | HGF | MAF | NBN | PIK3CB | RICTOR | TGFBR2 |
| ASXL1 | CCND2 | CUL4A | FGF12 | HNF1A | MAP2K1 | NF1 | PIK3R1 | RNF43 | TIPARP |
| ATM | CCND3 | CXCR4 | FGF14 | HRAS | MAP2K2 | NF2 | PIM1 | ROS1 | TNFAIP3 |
| ATR | CCNE1 | CYP17A1 | FGF19 | HSD3B1 | MAP2K4 | NFE2L2 | PMS2 | RPTOR | TNFRSF14 |
| ATRX | CD22 | DAXX | FGF23 | ID3 | MAP3K1 | NFKBIA | POLD1 | SDHA | TP53 |
| AURKA | CD274 | DDR1 | FGF3 | IDH1 | MAP3K13 | NKX2-1 | POLE | SDHB | TSC1 |
| AURKB | CD70 | DDR2 | FGF4 | IDH2 | MAPK1 | NOTCH1 | PPARG | SDHC | TSC2 |
| AXIN1 | CD79A | DIS3 | FGF6 | IGF1R | MCL1 | NOTCH2 | PPP2R1A | SDHD | TYRO3 |
| AXL | CD79B | DNMT3A | FGFR1 | IKBKE | MDM2 | NOTCH3 | PPP2R2A | SETD2 | U2AF1 |
| BAP1 | CDC73 | DOT1L | FGFR2 | IKZF1 | MDM4 | NPM1 | PRDM1 | SF3B1 | VEGFA |
| BARD1 | CDH1 | EED | FGFR3 | INPP4B | MED12 | NRAS | PRKAR1A | SGK1 | VHL |
| BCL2 | CDK12 | EGFR | FGFR4 | IRF2 | MEF2B | NT5C2 | PRKCI | SMAD2 | WHSC1 |
| BCL2L1 | CDK4 | EP300 | FH | IRF4 | MEN1 | NTRK1 | PTCH1 | SMAD4 | WHSC1L1 |
| BCL2L2 | CDK6 | EPHA3 | FLCN | IRS2 | MERTK | NTRK2 | PTEN | SMARCA4 | WT1 |
| BCL6 | CDK8 | EPHB1 | FLT1 | JAK1 | MET | NTRK3 | PTPN11 | SMARCB1 | XPO1 |
| BCOR | CDKN1A | EPHB4 | FLT3 | JAK2 | MITF | P2RY8 | PTPRO | SMO | XRCC2 |
| BCORL1 | CDKN1B | ERBB2 | FOXL2 | JAK3 | MKNK1 | PALB2 | QKI | SNCAIP | ZNF217 |
| BRAF | CDKN2A | ERBB3 | FUBP1 | JUN | MLH1 | PARK2 | RAC1 | SOCS1 | ZNF703 |
| BRCA1 | CDKN2B | ERBB4 | GABRA6 | KDM5A | MPL | PARP1 | RAD21 | SOX2 | |

TABLE 13
Genes with selected intronic regions for the detection of gene rearrangements,
one with 3′UTR, one gene with a promoter region and one ncRNA gene.

| ALK | BRCA1 | ETV4 | EZR | KIT | MYC | NUTM1 | RET | SLC34A2 |
|---|---|---|---|---|---|---|---|---|
| introns 18, | introns 2, | introns 5, | introns 9-11 | intron 16 | intron 1 | intron 1 | introns | intron 4 |
| 19 | 7, 8, 12, | 6 | | | | | 7-11 | |
| | 16, 19, 20 | | | | | | | |
| BCL2 | BRCA2 | ETV5 | FGFR1 | KMT2A (MLL) | NOTCH2 | PDGFRA | ROS1 | TERC |
| 3′UTR | intron 2 | introns 6, | intron 1, | introns | intron 26 | introns 7, | introns | ncRNA |
| | | 7 | 5, 17 | 6-11 | | 9, 11 | 31-35 | |
| BCR | CD74 | ETV6 | FGFR2 | MSH2 | NTRK1 | RAF1 | RSPO2 | TERT |
| introns 8, | introns | introns 5, | intron 1, | intron 5 | introns | introns | intron 1 | Promoter |
| 13, 14 | 6-8 | 6 | 17 | | 8-10 | 4-8 | | |
| BRAF | EGFR | EWSR1 | FGFR3 | MYB | NTRK2 | RARA | SDC4 | TMPRSS2 |
| introns | introns 7, | introns | intron 17 | intron 14 | Intron 12 | intron 2 | intron 2 | introns 1-3 |
| 7-10 | 15, 24-27 | 7-13 | | | | | | |

The F1CDx assay identifies various alterations in the gene and/or intron sequences, including substitutions, insertions/deletions, and CNAs. The F1CDx assay was previously identifies as having concordance with an externally validated NGS assay and the FOUNDATIONONE® (F1 LDT) assay. See FOUNDATIONONE® CDX™: Technical Information, Foundation Medicine, Inc., available at FoundationMedicine.com, last visited Mar. 25, 2019, which is incorporated by reference herein in its entirety.

II.B.1.f. MSK-IMPACT™

In some aspects, TMB status is assessed using the MSK-IMPACT™ assay. The MSK-IMPACT™ assay uses next-generation sequencing to analyze the mutation status of 468 genes. Target genes are captured and sequenced on an ILLUMINA HISEQ™ instrument. The MSK-IMPACT™ assay is approved by the US FDA for detection of somatic mutations and microsatellite instability in solid malignant neoplasms. The full list of 468 genes analyzed by the MSK-IMPACT™ assay is shown in Table 14. See Evaluation of Automatic Class III Designation for MSK-IMPACT (Integrated Mutation Profiling of Actionable Cancer Targets): Decision Summary, United States Food and Drug Administration, Nov. 15, 2017, available at accessdata.fda.gov.

TABLE 14
Genes analyzed by the MSK-IMPACT ™ assay.

| | | | | | | | | | |
|---|---|---|---|---|---|---|---|---|---|
| ABL1 | CALR | DDR2 | FGF19 | HIST3H3 | LYN | NKX2-1 | PPARG | RPTOR | STK19 |
| ACVR1 | CARD11 | DICER1 | FGF3 | HLA-A | MALT1 | NKX3-1 | PPM1D | RRAGC | STK40 |
| AGO2 | CARM1 | DIS3 | FGF4 | HLA-B | MAP2K1 | NOTCH1 | PPP2R1A | RRAS | SUFU |
| AKT1 | CASP8 | DNAJB1 | FGFR1 | HNF1A | MAP2K2 | NOTCH2 | PPP4R2 | RRAS2 | SUZ12 |
| AKT2 | CBFB | DNMT1 | FGFR2 | HOXB13 | MAP2K4 | NOTCH3 | PPP6C | RTEL1 | SYK |
| AKT3 | CBL | DNMT3A | FGFR3 | HRAS | MAP3K1 | NOTCH4 | PRDM1 | RUNX1 | TAP1 |
| ALK | CCND1 | DNMT3B | FGFR4 | ICOSLG | MAP3K13 | NPM1 | PRDM14 | RXRA | TAP2 |
| ALOX12B | CCND2 | DOT1L | FH | ID3 | MAP3K14 | NRAS | PREX2 | RYBP | TBX3 |
| AMER1 | CCND3 | DROSHA | FLCN | IDH1 | MAPK1 | NSD1 | PRKAR1A | SDHA | TCEB1 |
| ANKRD11 | CCNE1 | DUSP4 | FLT1 | IDH2 | MAPK3 | NTHL1 | PRKCI | SDHAF2 | TCF3 |
| APC | CD274 | E2F3 | FLT3 | IFNGR1 | MAPKAP1 | NTRK1 | PRKD1 | SDHB | TCF7L2 |
| AR | CD276 | EED | FLT4 | IGF1 | MAX | NTRK2 | PTCH1 | SDHC | TEK |
| ARAF | CD79A | EGFL7 | FOXA1 | IGF1R | MCL1 | NTRK3 | PTEN | SDHD | TERT |
| ARID1A | CD79B | EGFR | FOXL2 | IGF2 | MDC1 | NUF2 | PTP4A1 | SESN1 | TET1 |
| ARID1B | CDC42 | EIF1AX | FOXO1 | IKBKE | MDM2 | NUP93 | PTPN11 | SESN2 | TET2 |
| ARID2 | CDC73 | EIF4A2 | FOXP1 | IKZF1 | MDM4 | PAK1 | PTPRD | SESN3 | TGFBR1 |
| ARID5B | CDH1 | EIF4E | FUBP1 | IL10 | MED12 | PAK7 | PTPRS | SETD2 | TGFBR2 |
| ASXL1 | CDK12 | ELF3 | FYN | IL7R | MEF2B | PALB2 | PTPRT | SETD8 | TMEM127 |
| ASXL2 | CDK4 | EP300 | GATA1 | INHA | MEN1 | PARK2 | RAB35 | SF3B1 | TMPRSS2 |
| ATM | CDK6 | EPAS1 | GATA2 | INHBA | MET | PARP1 | RAC1 | SH2B3 | TNFAIP3 |
| ATR | CDK8 | EPCAM | GATA3 | INPP4A | MGA | PAX5 | RAC2 | SH2D1A | TNFRSF14 |
| ATRX | CDKN1A | EPHA3 | GLI1 | INPP4B | MITF | PBRM1 | RAD21 | SHOC2 | TOP1 |
| AURKA | CDKN1B | EPHA5 | GNA11 | INPPL1 | MLH1 | PDCD1 | RAD50 | SHQ1 | TP53 |
| AURKB | CDKN2A | EPHA7 | GNAQ | INSR | MPL | PDCD1LG2 | RAD51 | SLX4 | TP53BP1 |
| | p14ARF | | | | | | | | |
| AXIN1 | CDKN2A | EPHB1 | GNAS | IRF4 | MRE11A | PDGFRA | RAD51B | SMAD2 | TP63 |
| | p16INK4A | | | | | | | | |
| AXIN2 | CDKN2B | ERBB2 | GPS2 | IRS1 | MSH2 | PDGFRB | RAD51C | SMAD3 | TRAF2 |
| AXL | CDKN2C | ERBB3 | GREM1 | IRS2 | MSH3 | PDPK1 | RAD51D | SMAD4 | TRAF7 |
| B2M | CEBPA | ERBB4 | GRIN2A | JAK1 | MSH6 | PGR | RAD52 | SMARCA4 | TSC1 |
| BABAM1 | CENPA | ERCC2 | GSK3B | JAK2 | MSI1 | PHOX2B | RAD54L | SMARCB1 | TSC2 |
| BAP1 | CHEK1 | ERCC3 | H3F3A | JAK3 | MSI2 | PIK3C2G | RAF1 | SMARCD1 | TSHR |
| BARD1 | CHEK2 | ERCC4 | H3F3B | JUN | MST1 | PIK3C3 | RARA | SMO | U2AF1 |
| BBC3 | CIC | ERCC5 | H3F3C | KDM5A | MST1R | PIK3CA | RASA1 | SMYD3 | UPF1 |
| BCL10 | CREBBP | ERF | HGF | KDM5C | MTOR | PIK3CB | RB1 | SOCS1 | VEGFA |
| BCL2 | CRKL | ERG | HIST1H1C | KDM6A | MUTYH | PIK3CD | RBM10 | SOS1 | VHL |
| BCL2L1 | CRLF2 | ERRFI1 | HIST1H2BD | KDR | MYC | PIK3CG | RECQL | SOX17 | VTCN1 |
| BCL2L11 | CSDE1 | ESR1 | HIST1H3A | KEAP1 | MYCL1 | PIK3R1 | RECQL4 | SOX2 | WHSC1 |
| BCL6 | CSF1R | ETV1 | HIST1H3B | KIT | MYCN | PIK3R2 | REL | SOX9 | WHSC1L1 |
| BCOR | CSF3R | ETV6 | HIST1H3C | KLF4 | MYD88 | PIK3R3 | RET | SPEN | WT1 |
| BIRC3 | CTCF | EZH1 | HIST1H3D | KMT2A | MYOD1 | PIM1 | RFWD2 | SPOP | WWTR1 |
| BLM | CTLA-4 | EZH2 | HIST1H3E | KMT2B | NBN | PLCG2 | RHEB | SPRED1 | XIAP |
| BMPR1A | CTNNB1 | FAM175A | HIST1H3F | KMT2C | NCOA3 | PLK2 | RHOA | SRC | XPO1 |
| BRAF | CUL3 | FAM46C | HIST1H3G | KMT2D | NCOR1 | PMAIP1 | RICTOR | SRSF2 | XRCC2 |
| BRCA1 | CXCR4 | FAM58A | HIST1H3H | KNSTRN | NEGR1 | PMS1 | RIT1 | STAG2 | YAP1 |
| BRCA2 | CYLD | FANCA | HIST1H3I | KRAS | NF1 | PMS2 | RNF43 | STAT3 | YES1 |
| BRD4 | CYSLTR2 | FANCC | HIST1H3J | LATS1 | NF2 | PNRC1 | ROS1 | STAT5A | ZFHX3 |
| BRIP1 | DAXX | FAT1 | HIST2H3C | LATS2 | NFE2L2 | POLD1 | RPS6KA4 | STAT5B | |
| BTK | DCUN1D1 | FBXW7 | HIST2H3D | LMO1 | NFKBIA | POLE | RPS6KB2 | STK11 | |
| ABL1 | CALR | DDR2 | FGF19 | HIST3H3 | LYN | NKX2-1 | PPARG | RPTOR | STK19 |

II.B.1.g. NEOGENOMICS® NEOTYPE™ Assays

In some aspects, TMB is determined using a NEOGENOMICS® NEOTYOPE™ assay. In some aspects, the TMB is determined using a NEOTYPE™ Discovery Profile. In some aspects, the TMB is determined using a NEOTYPE Solid Tumor Profile. The NEOGENOMICS assays measure the number of non-synonymous DNA coding sequence changes per megabase of sequenced DNA.

II.B.1.h. ONCOMINE™ Tumor Mutation Load Assay

In some aspects, TMB is determined using a THERMOFISHER SCIENTIFIC® ONCOMINE™ Tumor Mutation assay. In some aspects, TMB is determined using a THERMOFISHER SCIENTIFIC® ION TORRENT™ ONCOMINE™ Tumor Mutation assay. The ION TORRENT™ ONCOMINE™ Tumor Mutation assay is a targeted NGS assay that quantitates somatic mutations to determine tumor mutation load. The assay covers 1.7 Mb of DNA. The full list of 408 genes analyzed by the THERMOFISHER SCIENTIFIC® ION TORRENT™ ONCOMINE™ Tumor Mutation assay is shown in Table 15 (see Iontorrent, Oncomine Tumor Mutation Load Assay Flyer, available at assets.thermofisher.com/TFS-Assets/CSD/Flyers/oncomine-tumor-mutation-load-assay-flyer.pdf, last visited Mar. 25, 2019).

TABLE 15
Genes analyzed by the THERMOFISHER SCIENTIFIC ® ION TORRENT ™ ONCOMINE ™ Tumor Mutation assay.

| | | | | | | | | | |
|---|---|---|---|---|---|---|---|---|---|
| 0082 | ATR | CSF1R | FAM123B | HRAS | LRP18 | MY8 | PDGFRB | RHOH | TCF7L1 |
| SEPT9 | ATRX | CSMD3 | FANCA | HSP90A81 | LTF | MYCLI | PER1 | RNASEL | TCF7L2 |
| 81RC2 | AURK8 | CTNNA1 | FANCC | HSP90AA1 | LTK | MYCN | PGAP3 | RNF2 | TCL1A |
| 81RC3 | AURKA | CTNNB1 | FANCD2 | ICK | M8D1 | MYD88 | PHOX28 | RNF213 | TET1 |
| 81RC5 | AURKC | CYLD | FANCF | IDH1 | MAF | MYH11 | PIK3C28 | ROS1 | TET2 |
| 8AI3 | AXL | CYP2C19 | FANCG | IDH2 | MAF8 | MYH9 | PIK3CA | RPS6KA2 | TFE3 |
| 8CL10 | BAP1 | CYP2D6 | FANCJ | IGF1R | MAGEA1 | NBN | PIK3CB | RRM1 | TGF8R2 |
| 8CL118 | BRAF | DAXX | FAS | IGF2 | MAGl1 | NCOAI | PIK3CD | RUNX1 | TGM7 |
| 8CL11A | CARD11 | DCC | FBXW7 | IGF2R | MALT1 | NCOA2 | PIK3CG | RUNXIT1 | TH8S1 |
| 8CL2 | CASCS | DDIT3 | FGFR1 | IKBKB | MAML2 | NCOA4 | PIK3R1 | SAMD9 | TIMP3 |
| 8CL2L1 | CBL | DDR2 | FGFR2 | IKBKE | MAP2K1 | NF1 | PIK3R2 | SBDS | TLR4 |
| 8CL2L2 | CCND1 | DEK | FGFR3 | IKZF1 | MAP2K2 | NF2 | PIM1 | SDHA | TLX1 |
| 8CL3 | CCND2 | DICER1 | FGFR4 | IL2 | MAP2K4 | NFE2L2 | PKHD1 | SDHB | TNFAIP3 |
| 8CL6 | CCNE1 | DNMT3A | FH | IL21R | MAP3K7 | NFK81 | PLAG1 | SDHD | TNFRSF14 |
| 8CL9 | CD79A | DPYD | FLCN | IL6ST | MAPK1 | NFK82 | PLCG1 | SETD2 | TNK2 |
| 8CR | CD79B | DST | FLl1 | IL7R | MAPK8 | NIN | PLEKHGS | SF3B1 | TOP1 |
| 8LM | CDC73 | EGFR | FLT1 | ING4 | MARK1 | NKX2-1 | PML | SGK1 | TP53 |
| 8LNK | CDH1 | EML4 | FLT3 | IRF4 | MARK4 | NLRP1 | PMS1 | SH2D1A | TPR |
| 8MPR1A | CDH11 | EP300 | FLT4 | IRS2 | MCL1 | NOTCH1 | PMS2 | SMAD2 | TRIM24 |
| 8RD3 | CDH2 | EP400 | FN1 | ITGA10 | MDM2 | NOTCH2 | POT1 | SMAD4 | TRIM33 |
| 8TK | CDH20 | EPHA3 | FOX01 | ITGA9 | MDM4 | NOTCH4 | POU5F1 | SMARCA4 | TRIP11 |
| 8U818 | CDH5 | EPHA7 | FOX03 | ITGB2 | MEN1 | NPM1 | PPARG | SMARCB1 | TRRAP |
| A8L2 | CDK12 | EPHB1 | FOXL2 | ITGB3 | MET | NRAS | PPP2R1A | SMO | TSC1 |
| ABL1 | CDK4 | EPHB4 | FOXP1 | JAK1 | MITF | NSD1 | PRDM1 | SMUG1 | TSC2 |
| ACVR2A | CDK6 | EPHB6 | FOXP4 | JAK2 | MLH1 | NTRK1 | PRKAR1A | SOCS1 | TSHR |
| ADAMTS2 | CDK8 | ERBB2 | FZR1 | JAK3 | MLL | NTRK3 | PRKDC | SOHO | U8R5 |
| AFF1 | CDKN2A | ERBB3 | G6PD | JUN | MLL2 | NUMA1 | PSIP1 | SOX11 | UGT1A1 |
| AFF3 | CDKN2B | ERBB4 | GATAI | KAT6A | MLL3 | NUP214 | PTCH1 | SOX2 | USP9X |
| AKAP9 | CDKN2C | ERCC1 | GATA2 | KAT6B | MLLT10 | NUP98 | PTEN | SRC | VHL |
| AKT1 | CEBPA | ERCC2 | GATA3 | KDM5C | MMP2 | P8RM1 | PTGS2 | SSXI | WAS |
| AKT2 | CHEK1 | ERCC3 | GDNF | KDM6A | MN1 | P8X1 | PTPN11 | STK11 | WHSCI |
| AKT3 | CHEK2 | ERCC4 | GNA11 | KEAP1 | MPL | PAK3 | PTPRD | STK36 | WRN |
| ALK | CIC | ERCC5 | GNAQ | KIT | MRE11A | PALB2 | PTPRT | SUFU | WT1 |
| APC | CKS1B | ERG | GNAS | KLF6 | MSH2 | PARP1 | RADSO | SYK | XP01 |
| AR | CMPK1 | ESR1 | GPR124 | KOR | MSH6 | PAX? | RAF1 | SYNE1 | XPA |
| ARID1A | COL1A1 | ETS1 | GRM8 | KRAS | MTC | PAX3 | RALGDS | T8X22 | XPC |
| ARID2 | CRBN | ETV1 | HCAR1 | LAMP1 | MTOT | PAX8 | RARA | TAF1 | XRCC2 |
| ARNT | CREB1 | ETV4 | HFN1A | LCK | MTR | PAXS | RB1 | TAF1L | ZNF384 |
| ASXL1 | CREBBP | EXT1 | HIF1A | LIFR | MTRR | PDE4DIP | RECOL4 | TAL1 | ZNF521 |
| ATF1 | CRKT | EXT2 | HLF | LPHN3 | MUCI | PDGF8 | REL | TCF12 | |
| ATM | CRTC1 | EZH2 | HOOK3 | LPP | MUTYH | PDGFRA | RET | TCF3 | |

II.B.1.i. NOVOGENE™ NOVOPM™ Assay

In some aspects, TMB is determined using a NOVOGENE™ NOVOPM™ assay. In some aspects, TMB is determined using a NOVOGENE™ NOVOPM™ Cancer Panel assay. The NOVOGENE™ NOVOPM™ Cancer Panel assay is a comprehensive NGS cancer panel that analyzes the complete coding regions of 548 genes and the introns of 21 genes, representing about 1.5 Mb of DNA, and that are relevant for the diagnosis and/or treatment of solid tumors according to the National Comprehensive Cancer Network (NCCN) guidelines and medical literature. The assay detects SNV, InDel, fusion, and copy number variation (CNV) genomic abnormalities.

II.B.1.j. Other TMB Assays

In some aspects, TMB is determined using a TMB assay provided by CARIS® Life Sciences. In some aspects, TMB is determined using the PESONALIS® ACE ImmunoID assay. In some aspects, TMB is determined using the PGDX® CANCERXOME™-R assay.

In yet another particular aspect, the genomic profiling detects all mutation types, i.e., single nucleotide variants, insertions/deletions (indels), copy number variations, and rearrangements, e.g., translocations, expression, and epigenetic markers.

Comprehensive gene panels often contain predetermined genes selected based on the type of tumor to be analyzed. Accordingly, the genomic profile used to measure TMB status can be selected based on the type of tumor the subject has. In one aspect, the genomic profile can include a set of genes particular to a solid tumor. In another aspect, the genomic profile can include a set of genes particular to hematologic malignancies and sarcomas.

In one aspect, the genomic profile comprises one or more genes selected from the group consisting of ABL1, BRAF, CHEK1, FANCC, GATA3, JAK2, MITF, PDCD1LG2, RBMO, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4 (C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG11, CRLF2, FANCL, GNA11, KDM5C, MSH6, PIK3CA, RPTOR, TBX3, ALK, BTK, CSFIR, FAS, GNA13, KDM6A, MTOR, PIK3CB, RUNX1, TERC, AMER1(FAM123B), C11orf30 (EMSY), CTCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2RIA, SLIT2, TP53, ASXL1, CD274, DNMT3A, FGF6, HNFA, KRAS, NFKB1A, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP90AA1, LYN, NOTCH2, PRKC1, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, APO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRF11, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STAT3, and any combination thereof. In other aspects, the TMB analysis further comprises identifying a genomic alteration in one or more of ETV4, TMPRSS2, ETV5, BCR, ETV, ETV6, and MYB.

In another aspect, the genomic profile comprises one or more genes selected from the group consisting of ABL1, 12B, ABL2, ACTB, ACVR1, ACVR1B, AGO2, AKT1, AKT2, AKT3, ALK, ALOX, ALOX12B, AMER1, AMER1(FAM123B or WTX), AMER1(FAM123B), ANKRD11, APC, APHIA, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXIN1, AXIN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1 (BACH1), BRSK1, BTG1, BTG2, BTK, BTLA, C11orf 30 (EMSY), C11orf30, C11orf30 (EMSY), CAD, CALR, CARD11, CARMI, CASP8, CBFB, CBL, CCND1, CCND2, CCND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKNV2Ap16INK4A, CDKN2B, CDKN2C, CEBPA, CENPA, CHD2, CHD4, CHEK1, CHEK2, CIC, CIITA, CKS1B, CPS1, CREBBP, CRKL, CRLF2, CSDE1, CSFIR, CSF3R, CTCF, CLTA-4, CTNNB1, CTNNA1, CTNNB1, CUL3, CUL4A, CUX1, CXCR4, CYLD, CYP17A1, CYSLTR2, DAXX, DCUNID1, DDR1, DDR2, DDX3X, DH2, DICER1, DIS3, DNAJB1, DNM2, DNMT1, DNMT3A, DNMT3B, DOTIL, DROSHA, DTX1, DUSP2, DUSP4, DUSP9, E2F3, EBF1, ECT2L, EED, EGFL7, EGFR, EIFIAX, EIF4A2, EIF4E, ELF3, ELP2, EML4, EML4-ALK, EP300, EPAS1, EPCAM, EPHA3, EPHA5, EPHA7, EPHB1, EPHB4, ERBB2, ERBB3, ERBB4, ERCC1, ERCC2, ERCC3, ERCC4, ERCC5, ERF, ERG, ERRF11, ERRF11, ESR1, ETS1, ETV1, ETV4, ETV5, ETV6, EWSR1, EXOSC6, EZH1, EZH2, FAF1, FAM175A, FAM46C, FAM58A, FANCA, FANCC, FANCD2, FANCE, FANCF, FANCG, FANC1, FANCL, FAS, FAS (TNFRSF6), FAT1, FBXO11, FBXO31, FBXW7, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLCN, FL11, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 (C17orf 39), GID4 (C17orf39), GLI1, GL11, GNA11, GNA12, GNA13, GNAQ, GNAS, GPR124, GPS2, GREM1, GRIN2A, GRM3, GSK3B, GTSE1, H3F3A, H3F3B, H3F3C, HDAC, HDAC4, HDAC7, Hedgehog, HER-2/NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNFA, HOXB13, HRAS, HSD3B1, HSP90AA1, ICK, ICOSLG, ID3, IDH1, IDH2, IFNGR1, IGF1, IGF1R, IGF2, IKBKE, IKZF, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST 3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT2C (MLL3), KMT2D, KMT2D (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K, MAP2K1 (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX, MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE 11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1(PD-1), PDCD11, PDCD1LG2, PDCD1LG2 (PD-L2), PDGFRA, PDGFRB, PDK1, PDPK1, PGR, PHF6, PHOX2B, PIK3C2B, PIK3C2G, PIK3C3, PIK3CA, PIK3CB, PIK3CD, PIK3CG, PIK3R1, PIK3R2, PIK3R3, PIM, PLCG2, PLK2, PMAIP1, PMS, PMS2, PNRC, POLD, POLE, POT1, PPARG, PPM1D, PPP2, PPP2R1A, PPP2R2A, PPP4R2, PPP6C, PRDM1, PRDM14, PREX2, PRKAR1A, PRKC1, PRKD1, PRKDC, PRSS8, PTCH, PTEN, PTP4A1, PTPN1, PTPN2, PTPN6 (SHP-1), PTPRD, PTPRO, PTPRS, PTPRT, QKI, RIA, RAB35, RAC1, RAC2, RAD21, RAD50, RAD51, RAD51B, RAD51C, RAD51D, RAD52, RAD54L, RAF1, RANBP2, RARA, RASA1, RASGEFIA, RB1, RBMO, RECQL, RECQL4, REL, RELN, RET, RFWD2, RHEB, RHOA, RICTOR, RIT1, RNF43, ROS1, RPS6KA4, RPS6 KB1, RPS6KB2, RPTOR, RRAGC, RRAS, RRAS2, RTEL1, RUNX1, RUNX1T1, RXRA, RYBP, S1PR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQI, SLIT2, SLX4, SMAD2, SMAD3, SMAD4, SMARCA1, SMARCA4, SMARCBM, SMARCD, SMC1A, SMC3, SMO, SMYD3, SNCAIP, SOCS1, SOCS2, SOCS3, SOS1, SOX0, SOX17, SOX2, SOX9, SPEN, SPOP, SPRED, SPTA1, SRC, SRSF2, STAG2, STAT3, STAT4, STAT5A, STAT5B, STAT6, STK11, STK19, STK40, SUFU, SUZ12, SYK, TAF1, TAP1, TAP2, TBL1XR1, TBX3, TCEB1, TCF3, TCF3 (E2A), TCF7L2, TCL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF1A, TNFRSF4, TNFRSF7, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1(MASET or NSD2), WHSC1L1, WISP3, WT1, WWTR1, XBP1, XIAP, XPO1, ARCC2, YAP1, YES1, YY1API, ZBTB2, ZFHX3, ZMYM3, ZNF217, ZNF24 (ZSCAN3), ZNF703, ZRSR2, 0082, SEPT9, 81RC2, 81RC3, 81RC5, 8AI3, 8CLIO, 8CL118, 8CL11A, 8CL2, 8CL2L1, 8CL2L2, 8CL3, 8CL6, 8CL9, 8CR, 8LM, 8LNK, 8MPRIA, 8RD3, 8TK, 8U818, A8L2, ACVR2A, ADAMTS2, AFF1, AFF3, AKAP9, ARNT, ATF1, AURK8, AURKC, CASCS, CDH11, CDH2, CDH20, CDH5, CMPK1, COLIA1, CRBN, CREB1, CRTC1, CSMD3, CYP2C19, CYP2D6, DCC, DDIT3, DEK, DPYD, DST, EP400, EXT1, EXT2, FAM123B, FANC1, FL11, FN1, FOX01, FOX03, FOXP4, FZR1, G6PD, GDNF, GRM8, HCAR1, HFNIA, HIFA, HLF, HOOK3, HSP90A81, ICK, IGF2R, IKBKB, IL2, IL21R, IL6ST, ING4, ITGA10, ITGA9, ITGB2, ITGB3, KAT6A, KAT6B, KLF6, KOR, LCK, LIFR, LPHN3, LPP, LRP18, LTF, M8D1, MAF8, MAGEA1, MAG11, MAML2, MAPK8, MARK1, MARK4, MLL, MLL2, MLL3, MLLT0, MMP2, MN1, MTC, MTOT, MTR, MTRR, MUC1, MY8, MYH11, MYH9, NCOA1, NCOA2, NCOA4, NFK81, NFK82, NIN, NLRP1, NUMA1, NUP214, P8RM1, P8X1, PAX?, PAX3, PAX8, PAXS, PDE4DIP, PDGF8, PER1, PGAP3, PHOX28, PIK3C28, PKHD1, PLAG1, PLCG1, PLEKHGS, PML, POU5F1, PSIP1, PTGS2, RADSO, RALGDS, RHOH, RNASEL, RNF2, RNF213, RPS6KA2, RRM1, SAMD9, SBDS, SMUG1, SOHO, SOX11, SSX1, STK36, SYNE1, T8X22, TAF1L, TAL1, TCF12, TCF7L1, TFE3, TGF8R2, TGM7, TH8S1, TIMP3, TLR4, TLX1, TNK2, TPR, TRIM24, TRIM33, TRIP11, TRRAP, U8R5, UGTIA1, USP9X, WAS, WRN, XP01, XPA, XPC, ZNF384, ZNF521, and any combination thereof.

In another aspect, the genomic profiling assay comprises at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, or at least about 300 genes selected from the group consisting of ABL1, 12B, ABL2, ACTB, ACVR1, ACVR1B, AGO2, AKT1, AKT2, AKT3, ALK, ALOX, ALOX12B, AMER1, AMER1(FAM123B or WTX), AMER1(FAM123B), ANKRD11, APC, APHIA, AR, ARAF, ARFRP1, ARHGAP26 (GRAF), ARID1A, ARID1B, ARID2, ARID5B, ARv7, ASMTL, ASXL1, ASXL2, ATM, ATR, ATRX, AURKA, AURKB, AXIN1, AXIN2, AXL, B2M, BABAM1, BAP1, BARD1, BBC3, BCL10, BCL11B, BCL2, BCL2L1, BCL2L11, BCL2L2, BCL6, BCL7A, BCOR, BCORL1, BIRC3, BLM, BMPR1A, BRAF, BRCA1, BRCA2, BRD4, BRIP1, BRIP1(BACH1), BRSK1, BTG1, BTG2, BTK, BTLA, C11orf 30 (EMSY), C11orf30, C11orf30 (EMSY), CAD, CALR, CARD11, CARMI, CASP8, CBFB, CBL, CCND1, CCND2, CCND3, CCNE1, CCT6B, CD22, CD274, CD274 (PD-L1), CD276, CD36, CD58, CD70, CD79A, CD79B, CDC42, CDC73, CDH1, CDK12, CDK4, CDK6, CDK8, CDKN1A, CDKN1B, CDKN2A, CDKN2Ap14ARF, CDKN2Ap16INK4A, CDKN2B, CDKN2C, CEBPA, CENPA, CHD2, CHD4, CHEK1, CHEK2, CIC, CHTA, CKS1B, CPS1, CREBBP, CRKL, CRLF2, CSDE1, CSFIR, CSF3R, CTCF, CLTA-4, CTNNB1, CTNNA1, CTNNB1, CUL3, CUL4A, CUX1, CXCR4, CYLD, CYP17A1, CYSLTR2, DAXX, DCUNID1, DDR1, DDR2, DDX3X, DH2, DICER1, DIS3, DNAJB1, DNM2, DNMT1, DNMT3A, DNMT3B, DOT1L, DROSHA, DTX, DUSP2, DUSP4, DUSP9, E2F3, EBF1, ECT2L, EED, EGFL7, EGFR, EIFIAX, EIF4A2, EIF4E, ELF3, ELP2, EML4, EML4-ALK, EP300, EPAS, EPCAM, EPHA3, EPHA5, EPHA7, EPHB1, EPHB4, ERBB2, ERBB3, ERBB4, ERCC1, ERCC2, ERCC3, ERCC4, ERCC5, ERF, ERG, ERRF11, ERRF11, ESR1, ETS1, ETV1, ETV4, ETV5, ETV6, EWSR1, EXOSC6, EZH1, EZH2, FAF, FAM75A, FAM46C, FAM58A, FANCA, FANCC, FANCD2, FANCE, FANCF, FANCG, FANC1, FANCL, FAS, FAS (TNFRSF6), FAT1, FBXO11, FBXO31, FBXW7, FGF1, FGF0, FGF12, FGF14, FGF19, FGF2, FGF23, FGF3, FGF4, FGF5, FGF6, FGF7, FGF8, FGF9, FGFR1, FGFR2, FGFR3, FGFR4, FH, FHIT, FLCN, FLI1, FLT1, FLT3, FLT4, FLYWCH1, FOXA1, FOXL2, FOXO1, FOXO3, FOXP1, FRS2, FUBP1, FYN, GABRA6, GADD45B, GATA1, GATA2, GATA3, GATA4, GATA6, GEN1, GID4 (C17orf 39), GID4 (C17orf39), GLI1, GL11, GNA11, GNA12, GNA13, GNAQ, GNAS, GPR124, GPS2, GREMI, GRIN2A, GRM3, GSK3B, GTSE1, H3F3A, H3F3B, H3F3C, HDAC1, HDAC4, HDAC7, Hedgehog, HER-2/NEU; ERBB2, HGF, HIST1H1C, HIST1H1D, HIST1H1E, HIST1H2AC, HIST1H2AG, HIST1H2AL, HIST1H2AM, HIST1H2BC, HIST1H2BD, HIST1H2BJ, HIST1H2BK, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST2H3C, HIST2H3D, HIST3H3, HLA-A, HLA-B, HNFA, HOXB13, HRAS, HSD3B1, HSP90AA1, ICK, ICOSLG, ID3, IDH1, IDH2, IFNGR1, IGF1, IGF1R, IGF2, IKBKE, IKZF, IKZF2, IKZF3, IL10, IL7R, INHA, INHBA, INPP4A, INPP4B, INPP5D (SHIP), INPPL1, INSR, IRF1, IRF2, IRF4, IRF8, IRS1, IRS2, JAK1, JAK2, JAK3, JARID2, JUN, K14, KAT6A (MYST 3), KAT6A (MYST3), KDM2B, KDM4C, KDM5A, KDM5C, KDM6A, KDR, KEAP1, KEL, KIF5B, KIT, KLF4, KLHL6, KMT2A, KMT2A (MLL), KMT2B, KMT2C, KMT2C (MLL3), KMT2D, KMT2D (MLL2), KNSTRN, KRAS, LAMP1, LATS1, LATS2, LEF1, LMO1, LRP1B, LRRK2, LTK, LYN, LZTR1, MAF, MAFB, MAGED1, MAGI2, MALT1, MAP2K, MAP2K (MEK1), MAP2K2, MAP2K2 (MEK2), MAP2K4, MAP3, MAP3K1, MAP3K13, MAP3K14, MAP3K6, MAP3K7, MAPK1, MAPK3, MAPKAP1, MAX, MCL1, MDC1, MDM2, MDM4, MED12, MEF2B, MEF2C, MEK1, MEN1, MERTK, MET, MGA, MIB1, MITF, MKI67, MKNK1, MLH1, MLLT3, MPL, MRE 11A, MRE11A, MSH2, MSH3, MSH6, MSI1, MSI2, MST1, MST1R, MTAP, MTOR, MUTYH, MYC, MYCL, MYCL (MYC L1), MYCL (MYCL1), MYCL1, MYCN, MYD88, MYO18A, MYOD1, NBN, NCOA3, NCOR1, NCOR2, NCSTN, NEGR1, NF1, NF2, NFE2L2, NFKB1A, NKX2-1, NKX3-1, NOD1, NOTCH1, NOTCH2, NOTCH3, NOTCH4, NPM1, NRAS, NRG1, NSD1, NT5C2, NTHL1, NTRK1, NTRK2, NTRK3, NUF2, NUP93, NUP98, P2RY8, PAG1, PAK1, PAK3, PAK7, PALB2, PARK2, PARP1, PARP2, PARP3, PASK, PAX3, PAX5, PAX7, PBRM1, PC, PCBP1, PCLO, PDCD1, PDCD1(PD-1), PDCD11, PDCD1LG2, PDCD1LG2 (PD-L2), PDGFRA, PDGFRB, PDK1, PDPK1, PGR, PHF6, PHOX2B, PIK3C2B, PIK3C2G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, POT1, PPARG, PPMID, PPP2, PPP2R1A, PPP2R2A, PPP4R2, PPP6C, PRDM1, PRDM14, PREX2, PRKAR1A, PRKC1, PRKD1, PRKDC, PRSS8, PTCH1, PTEN, PTP4A1, PTPN1l, PTPN2, PTPN6 (SHP-1), PTPRD, PTPRO, PTPRS, PTPRT, QKI, RIA, RAB35, RAC1, RAC2, RAD21, RAD50, RAD51, RAD51B, RAD51C, RAD51D, RAD52, RAD54L, RAF1, RANBP2, RARA, RASA1, RASGEFA, RB1, RBM10, RECQL, RECQL4, REL, RELN, RET, RFWD2, RHEB, RHOA, RICTOR, RIT1, RNF43, ROS1, RPS6KA4, RPS6 KB1, RPS6KB2, RPTOR, RRAGC, RRAS, RRAS2, RTEL1, RUNX1, RUNX1T1, RXRA, RYBP, S1PR2, SDHA, SDHAF2, SDHB, SDHC, SDHD, SERP2, SESN1, SESN2, SESN3, SETBP1, SETD2, SETD8, SF3B1, SGK1, SH2B3, SH2D1A, SHOC2, SHQ1, SLIT2, SLX4, SMAD2, SMAD3, SMAD4, SMARCA1, SMARCA4, SMARCB1, SMARCDM, SMCIA, SMC3, SMO, SMYD3, SNCAIP, SOCS1, SOCS2, SOCS3, SOS1, SOX10, SOX17, SOX2, SOX9, SPEN, SPOP, SPRED1, SPTA1, SRC, SRSF2, STAG2, STAT3, STAT4, STAT5A, STAT5B, STAT6, STKI1, STK19, STK40, SUFU, SUZ12, SYK, TAF1, TAP1, TAP2, TBL1XR1, TBX3, TCEB1, TCF3, TCF3 (E2A), TCF7L2, TCL1A (TCL1), TEK, TERC, TERT, TERT Promoter, TET1, TET2, TFRC, TGFBR1, TGFBR2, TIPARP, TLL2, TMEM127, TMEM30A, TMPRSS2, TMSB4XP8 (TMSL3), TNFAIP3, TNFRSF1A, TNFRSF4, TNFRSF7, TOP1, TOP2A, TP53, TP53BP1, TP63, TRAF2, TRAF3, TRAF5, TRAF7, TSC1, TSC2, TSHR, TUSC3, TYK2, TYRO3, U2AF1, U2AF2, UPF1, VEGFA, VHL, VTCN1, WDR90, WHSC1, WHSC1(MMSET or NSD2), WHSCILI, WISP3, WT1, WWTR1, XBP1, MAP, XPO1, XRCC2, YAP1, YES1, YY1API, ZBTB2, ZFHX3, ZMYM3, ZNF217, ZNF24 (ZSCAN3), ZNF703, ZRSR2, 0082, SEPT9, 81RC2, 81RC3, 81RC5, 8AI3, 8CL10, 8CL118, 8CL11A, 8CL2, 8CL2L1, 8CL2L2, 8CL3, 8CL6, 8CL9, 8CR, 8LM, 8LNK, 8MPRIA, 8RD3, 8TK, 8U818, A8L2, ACVR2A, ADAMTS2, AFF1, AFF3, AKAP9, ARNT, ATF1, AURK8, AURKC, CASCS, CDH11, CDH2, CDH20, CDH5, CMPK1, COL1A1, CRBN, CREB1, CRTC1, CSMD3, CYP2C19, CYP2D6, DCC, DDIT3, DEK, DPYD, DST, EP400, EXT1, EXT2, FAM123B, FANC1, FLU, EN1, FOX01, FOX03, FOXP4, FZR1, G6PD, GDNF, GRM8, HCAR1, HFN1A, HIF1A, HLF, HOOK3, HSP90A81, ICK, IGF2R, IKBKB, IL2, IL21R, IL6ST, ING4, ITGA10, ITGA9, ITGB2, ITGB3, KAT6A, KAT6B, KLF6, KOR, LCK, LIFR, LPHN3, LPP, LRP18, LTF, M8D1, MAF8, MAGEA1, MAG11, MAML2, MAPK8, MARK1, MARK4, MLL, MLL2, MLL3, MLLT10, MA&P2, MN1, MTC, MTOT, MTR, MTRR, MUC1, MY8, MYH11, MYH9, NCOA1, NCOA2, NCOA4, NFK81, NFK82, NIN, NLRP1, NUMA1, NUP214, P8RM1, P8X1, PAX?, PAX3, PAX8, PAXS, PDE4DIP, PDGF8, PER1, PGAP3, PHOX28, PIK3C28, PKHD1, PLAG1, PLCG1, PLEKHGS, PML, POU5F1, PSIP1, PTGS2, RADSO, RALGDS, RHOH, RNASEL, RNF2, RNF213, RPS6KA2, RRM1, SAMD9, SBDS, SMUG1, SOHO, SOX11, SSX1, STK36, SYNE1, T8X22, TAFIL, TAL1, TCF12, TCF7L1, TFE3, TGF8R2, TGM7, TH8S1, TIMP3, TLR4, TLX1, TNK2, TPR, TRIM24, TRIM33, TRIP11, TRRAP, U8R5, UGT1A1, USP9X, WAS, WRN, XP01, XPA, XPC, ZNF384, ZNF521, and any combination thereof.

In another aspect, the genomic profile comprises one or more genes selected from the genes listed in Tables 2-15.

### II.B.2. TMB Status

In one aspect, TMB status based on genomic profiling is highly correlated with TMB status based on whole-exome or whole-genome sequencing. Evidence provided herein shows that the use of genomic profiling assays, such as the F1CDx assay, have concordance with whole-exome and/or whole genome sequencing assays. These data support the use of genomic profiling assays as a more efficient means of measuring TMB status, without forfeiting the prognostic qualities of TMB status.

TMB can be measured using a tissue biopsy sample or, alternatively, circulating tumor DNA (ctDNA), cfDNA (cell-free DNA), and/or a liquid biopsy sample. ctDNA can be used to measure TMB status according to whole-exome or whole-genome sequencing or genomic profiling using available methodologies, e.g., GRAIL, Inc.

In some aspects, a subject is identified as suitable for an anti-PD-1 therapy, as disclosed herein, based on the measurement of TMB status and identification of a high TMB. In some aspects, a TMB score is calculated as the total number of nonsynonymous missense mutations in a tumor, as measured by whole exome sequencing or whole genome sequencing. In one aspect, the high TMB has a score of at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500. In another aspect, the high TMB has a score of at least 215, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, or at least 250. In a particular aspect, the high TMB has a score of at least 243. In other aspects, the high TMB has a score of at least 244. In some aspects, the high TMB has a score of at least 245. In other aspects, the high TMB has a score of at least 246. In other aspects, the high TMB has a score of at least 247. In other aspects, the high TMB has a score of at least 248. In other aspects, the high TMB has a score of at least 249. In other aspects, the high TMB has a score of at least 250. In other aspects, the high TMB has a score of any integer between 200 and 300 or higher. In other aspects, the high TMB has a score of any integer between 210 and 290 or higher. In other aspects, the high TMB has a score of any integer between 220 and 280 or higher. In other aspects, the high TMB has a score of any integer between 230 and 270 or higher. In other aspects, the high TMB has a score of any integer between 235 and 265 or higher.

Alternatively, the high TMB can be a relative value rather than an absolute value. In some aspects, the subject's TMB status is compared to a reference TMB value. In one aspect, the subject's TMB status is within the highest fractile of the reference TMB value. In another aspect, the subject's TMB status is within the top tertile of the reference TMB value.

In some aspects, TMB status is expressed as the number of mutations per sample, per cell, per exome, or per length of DNA (e.g., Mb). In some aspects, a tumor has a high TMB status if the tumor has at least about 50 mutations/tumor, at least about 55 mutations/tumor, at least about 60 mutations/tumor, at least about 65 mutations/tumor, at least about 70 mutations/tumor, at least about 75 mutations/tumor, at least about 80 mutations/tumor, at least about 85 mutations/tumor, at least about 90 mutations/tumor, at least about 95 mutations/tumor, at least about 100 mutations/tumor, at least about 105 mutations/tumor, at least about 110 mutations/tumor, at least about 115 mutations/tumor, or at least about 120 mutations/tumor. In some aspects, a tumor has a high TMB status if the tumor has at least about 125 mutations/tumor, at least about 150 mutations/tumor, at least about 175 mutations/tumor, at least about 200 mutations/tumor, at least about 225 mutations/tumor, at least about 250 mutations/tumor, at least about 275 mutations/tumor, at least about 300 mutations/tumor, at least about 350 mutations/tumor, at least about 400 mutations/tumor, or at least about 500 mutations/tumor. In one particular aspect, a tumor has a high TMB status if the tumor has at least about 100 mutations/tumor.

In some aspects, a tumor has a high TMB status if the tumor has at least about 5 mutations per megabase of genes, e.g., genome sequenced according to a TMB assay, e.g., genome sequenced according to a FOUNDATIONONE® CDX™ assay, (mutations/Mb), at least about 6 mutations/Mb, at least about 7 mutations/Mb, at least about 8 mutations/Mb, at least about 9 mutations/Mb, at least about 10 mutations/Mb, at least about 11 mutations/Mb, at least about 12 mutations/Mb, at least about 13 mutations/Mb, at least about 14 mutations/Mb, at least about 15 mutations/Mb, at least about 20 mutations/Mb, at least about 25 mutations/Mb, at least about 30 mutations/Mb, at least about 35 mutations/Mb, at least about 40 mutations/Mb, at least about 45 mutations/Mb, at least about 50 mutations/Mb, at least about 75 mutations/Mb, or at least about 100 mutations/Mb. In certain aspects, a tumor has a high TMB status if the tumor has at least about 5 mutations/Mb. In certain aspects, a tumor has a high TMB status if the tumor has at least about 10 mutations/Mb. In some aspects, a tumor has a high TMB status if the tumor has at least about 11 mutations/Mb. In some aspects, a tumor has a high TMB status if the tumor has at least about 12 mutations/Mb. In some aspects, a tumor has a high TMB status if the tumor has at least about 13 mutations/Mb. In some aspects, a tumor has a high TMB status if the tumor has at least about 14 mutations/Mb. In certain aspects, a tumor has a high TMB status if the tumor has at least about 15 mutations/Mb.

Because the number of mutations varies by tumor type and other ways (see Q4 and Q5), the values associated with “TMB high” and “TMB low” can differ across tumor types.

### II.C. Antibodies

The present disclosure is directed to methods for treating a human subject afflicted with a cancer comprising administering to the subject a PD-1 inhibitor, e.g., an anti-PD-1 antibody or an anti-PD-L1 antibody. In some aspects, the subject is administered an anti-PD-1 monotherapy, e.g., wherein the subject is not administered one or more additional anti-cancer agent. In some aspects, the subject is administered a combination therapy, e.g., wherein the subject is administered an anti-PD-1 antibody and one or more additional anti-cancer agents. In certain aspects, the subject is administered a combination therapy comprising an anti-PD-1 antibody and an anti-CTLA-4 antibody.

In other aspects of the present disclosure, an anti-PD-L1 antibody is substituted for the anti-PD-1 antibody. In certain aspects, the methods comprise administering an anti-PD-L1 antibody to a subject. In some aspects, the subject is administered an anti-PD-L1 monotherapy. In some aspects, the subject is administered a combination therapy comprising an anti-PD-L1 antibody and a second anti-cancer agent, e.g., an anti-CTLA-4 antibody.

### II.C.1. Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANBO11; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one aspect, the anti-PD-1 antibody is administered at a dose of about 5 mg/kg body weight about once every 3 weeks. In another aspect, the anti-PD-1 antibody, e.g., nivolumab, is administered at a dose of about 3 mg/kg body weight about once every 2 weeks. In other aspects, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some aspects, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg. In one aspect, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another aspects, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some aspects, the anti-PD-1 antibody is administered as a flat dose of about 200 mg at about once every 3 weeks. In other aspects, the anti-PD-1 antibody is administered as a flat dose of about 200 mg at about once every 2 weeks. In other aspects, the anti-PD-1 antibody is administered as a flat dose of about 240 mg at about once every 2 weeks. In certain aspects, the anti-PD-1 antibody is administered as a flat dose of about 480 mg at about once every 4 weeks.

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks.

### II.C.2. Anti-PD-L1 Antibodies Useful for the Disclosure

In certain aspects, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15 suppl):TPS3113 (2018)), and CK-301 (Checkpoint Therapeutics; see Gorelik et al., AACR:Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular aspect, the anti-PD-L1 antibody is durvalumab. In other aspects, the anti-PD-L1 antibody is avelumab. In some aspects, the anti-PD-L1 antibody is atezolizumab.

In some aspects, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody is administered at a dose of about 15 mg/kg body weight at about once every 3 weeks. In other aspects, the anti-PD-L1 antibody is administered at a dose of about 10 mg/kg body weight at about once every 2 weeks.

In other aspects, the anti-PD-L1 antibody useful for the present disclosure is a flat dose. In some aspects, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody is administered as a flat dose of about 1200 mg at about once every 3 weeks. In other aspects, the anti-PD-L1 antibody is administered as a flat dose of about 800 mg at about once every 2 weeks. In other aspects, the anti-PD-L1 antibody is administered as a flat dose of about 840 mg at about once every 2 weeks.

In some aspects, atezolizumab is administered as a flat dose of about 1200 mg once about every 3 weeks. In some aspects, atezolizumab is administered as a flat dose of about 800 mg once about every 2 weeks. In some aspects, atezolizumab is administered as a flat dose of about 840 mg once about every 2 weeks.

In some aspects, avelumab is administered as a flat dose of about 800 mg once about every 2 weeks.

In some aspects, durvalumab is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

### II.C.3. Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (ka) of at least about 10³, about 10⁴, or about 10⁵ m⁻¹ s⁻¹; (c) a kinetic disassociation constant (kₐ) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab.

In particular aspects, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

In some aspects, the antibody that binds CTLA-4 is non-fucosylated. In some aspects, the antibody that binds CTLA-4 is a non-fucosylated antibody that comprises the 6 CDRs of ipilimumab. In some aspects, the antibody that binds CTLA-4 comprises the heavy chain variable region of ipilimumab and the light chain variable region of ipilimumab. In some aspects, the antibody that binds CTLA-4 is a non-fucosylated variant of ipilimumab. In some aspects, the antibody that binds CTLA-4 is a non-fucosylated anti-CTLA-4 antibody disclosed in International Publication No. WO 14/089113, which is incorporated by reference herein in its entirety.

In some aspects, the antibody is an activatable antibody disclosed in International Publication No. WO 18/085555 that when activated binds human CTLA-4. In some aspects, the activatable antibody comprises (a) a variable heavy domain and (b) a light chain, wherein the light chain comprises (i) a masking moiety (MM), (ii) a cleavable moiety (CM), and (iii) a variable light domain (VL). In some aspects, the light chain has the structural arrangement from N-terminus to C-terminus as follows: MM-CM-VL. In some aspects, the masking moiety inhibits binding of the antibody to CTLA-4, and the cleavable moiety is cleaved at the site of a tumor, removing the masking moiety, and activating the anti-CTLA-4 antibody. In some aspects, the antibody that binds CTLA-4 comprises (a) a variable heavy domain, comprising the variable heavy chain CDRs of ipilimumab; and (b) a light chain, wherein the light chain comprises (i) a masking moiety, (ii) a cleavable moiety, and (iii) a variable light domain, comprising the variable light chain CDRs of ipilimumab. In some aspects, the antibody that binds CTLA-4 comprises (a) the variable heavy domain of ipilimumab; and (b) a light chain, wherein the light chain comprises (i) a masking moiety, (ii) a cleavable moiety, and (iii) the variable light domain of ipilimumab. In some aspects, the light chain has the structural arrangement from N-terminus to C-terminus as follows: MM-CM-VL. In some aspects, the antibody that binds CTLA-4 comprises any antibody disclosed in International Publication No. WO 18/085555, which is incorporated by reference herein in its entirety.

In some aspects, the antibody that binds CTLA-4 is modified to enhance its activity in an acidic environment and/or to enhance its affinity for CTLA-4 in an acidic environment, as disclosed in International Publication No. WO 2020/214748. In some aspects, the antibody that binds CTLA-4 is modified to enhance its activity in an acidic environment. In some aspects, the antibody that binds CTLA-4 is modified to enhance its affinity for CTLA-4 in an acidic environment. In some aspects, the modified anti-CTLA-4 antibody comprises one or more mutations in the variable domain that enhance target binding at low/acidic pH (e.g., pH 6.0) compared with neutral pH (e.g., pH 7.4). In some aspects, the modified anti-CTLA-4 antibody comprises (a) a heavy chain variable region comprising (i) a histidine (H) substitution at one, two, three or more non-histidine positions, or (ii) at least one histidine residue in two or more of HC-CDR1, HC-CDR2, and HC-CDR3 of the anti-CTLA-4 antibody; (b) a light chain variable region comprising (i) a LC-CDR1 sequence having an acidic residue (e.g., aspartic acid or glutamic acid) substituted at one, two, three or more positions that were not previously aspartic acid or glutamic acid, or (ii) having one, two, three or more aspartic acid or glutamic acid residues in LC-CDRI; or (c) both (a) and (b). In some aspects of these aspects, the anti-CLTA-4 antibody is ipiliumumab or tremelimumab. In some aspects the anti-CTLA-4 antibody comprises an anti-CTLA-4 antibody disclosed in International Publication No. WO 2020/214748, which is incorporated by reference herein in its entirety.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding portion thereof cross-competes with ipilimumab and/or tremelimumab for binding to human CTLA-4.

In some aspects, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some aspects, the anti-CTLA-4 antibody or antigen-binding portion thereof is administered at a dose of 1 mg/kg or 3 mg/kg body weight once every 3, 4, 5, or 6 weeks. In one aspect, the anti-CTLA-4 antibody or antigen-binding portion thereof is administered at a dose of 3 mg/kg body weight once every 2 weeks. In another aspect, the anti-PD-1 antibody or antigen-binding portion thereof is administered at a dose of 1 mg/kg body weight once every 6 weeks.

In some aspects, the anti-CTLA-4 antibody or antigen-binding portion thereof is administered as a flat dose. In some aspects, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one aspect, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another aspect, the anti-CTLA-4 antibody or antigen-binding portion thereof is administered as a flat dose about once every 1, 2, 3, 4, 5, 6, 7, or 8 weeks.

In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses.

### II.C.4. Combination Therapies

In certain aspects, the anti-PD-1 antibody, the anti-PD-L1 antibody, and/or the anti-CTLA-4 antibody are administered at a therapeutically effective amount. In some aspects, the method comprises administering a therapeutically effective amount of anti-PD-1 antibody and an anti-CTLA-4 antibody. In other aspects, the method comprises administering a therapeutically effective amount of anti-PD-L1 antibody and an anti-CTLA-4 antibody. Any anti-PD-1, anti-PD-L1, or anti-CTLA-4 antibody disclosed herein can be used in the method. In certain aspects, the anti-PD-1 antibody comprises nivolumab. In some aspects, the anti-PD-1 antibody comprises pembrolizumab. In some aspects, the anti-PD-L1 antibody comprises atezolizumab. In some aspects, the anti-PD-L1 antibody comprises durvalumab. In some aspects, the anti-PD-L1 antibody comprises avelumab. In some aspects, the anti-CTLA-4 antibody comprises ipilimumab. In some aspects, the anti-CTLA-4 antibody comprises ipilimumab tremelimumab.

In some aspects, the (a) anti-PD-1 antibody or the anti-PD-L1 antibody and the (b) anti-CTLA-4 antibody are each administered once about every 2 weeks, once about every 3 weeks, once about every 4 weeks, once about every 5 weeks, or once about every 6 weeks. In some aspects, the anti-PD-1 antibody or the anti-PD-L1 antibody is administered once about every 2 weeks, once about every 3 weeks or once about every 4 weeks, and the anti-CTLA-4 antibody is administered once about every 6 weeks. In some aspects, the anti-PD-1 antibody or anti-PD-L1 antibody is administered on the same day as the anti-CTLA-4 antibody. In some aspects, the anti-PD-1 antibody or the anti-PD-L1 antibody is administered on a different day than the anti-CTLA-4 antibody.

In some aspects, the anti-CTLA-4 antibody is administered at a dose ranging from about 0.1 mg/kg to about 20.0 mg/kg body weight once about every 2, 3, 4, 5, 6, 7, or 8 weeks. In some aspects, the anti-CTLA-4 antibody is administered at a dose of about 0.1 mg/kg, about 0.3 mg/kg, about 0.6 mg/kg, about 0.9 mg/kg, about 1 mg/kg, about 3 mg/kg, about 6 mg/kg, about 9 mg/kg, about 10 mg/kg, about 12 mg/kg, about 15 mg/kg, about 18 mg/kg, or about 20 mg/kg. In certain aspects, the anti-CTLA-4 antibody is administered at a dose of about 1 mg/kg once about every 4 weeks. In some aspects, the anti-CTLA-4 antibody is administered at a dose of about 1 mg/kg once about every 6 weeks.

In some aspects, the anti-CTLA-4 antibody is administered at a flat dose. In some aspects, the anti-CTLA-4 antibody is administered at a flat dose ranging from at least about 40 mg to at least about 1600 mg. In some aspects, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some aspects, the CTLA-4 antibody is administered at a flat dose of at least about 220 mg, at least about 230 mg, at least about 240 mg, at least about 250 mg, at least about 260 mg, at least about 270 mg, at least about 280 mg, at least about 290 mg, at least about 300 mg, at least about 320 mg, at least about 360 mg, at least about 400 mg, at least about 440 mg, at least about 480 mg, at least about 520 mg, at least about 560 mg, or at least about 600 mg. In some aspects, the CTLA-4 antibody is administered at a flat dose of at least about 640 mg, at least about 720 mg, at least about 800 mg, at least about 880 mg, at least about 960 mg, at least about 1040 mg, at least about 1120 mg, at least about 1200 mg, at least about 1280 mg, at least about 1360 mg, at least about 1440 mg, or at least about 1600 mg. In some aspects, the anti-CTLA-4 antibody is administered in a flat dose at least once about every 2, 3, 4, 5, 6, 7, or 8 weeks.

In certain aspects, the anti-PD-1 antibody is administered at a dose of about 2 mg/kg once about every 3 weeks and the anti-CTLA-4 antibody is administered at a dose of about 1 mg/kg once about every 6 weeks. In some aspects, the anti-PD-1 antibody is administered at a dose of about 3 mg/kg once about every 2 weeks and the anti-CTLA-4 antibody is administered at a dose of about 1 mg/kg once about every 6 weeks. In some aspects, the anti-PD-1 antibody is administered at a dose of about 6 mg/kg once about every 4 weeks and the anti-CTLA-4 antibody is administered at a dose of about 1 mg/kg once about every 6 weeks.

In certain aspects, the anti-PD-1 antibody is administered at a flat dose of about 200 mg once about every 3 weeks and the anti-CTLA-4 antibody is administered at a dose of about 1 mg/kg once about every 6 weeks. In some aspects, the anti-PD-1 antibody is administered at a flat dose of about 200 mg once about every 2 weeks and the anti-CTLA-4 antibody is administered at a dose of about 1 mg/kg once about every 6 weeks. In some aspects, the anti-PD-1 antibody is administered at a flat dose of about 240 mg once about every 2 weeks and the anti-CTLA-4 antibody is administered at a dose of about 1 mg/kg once about every 6 weeks. In some aspects, the anti-PD-1 antibody is administered at a flat dose of about 480 mg once about every 4 weeks and the anti-CTLA-4 antibody is administered at a dose of about 1 mg/kg once about every 6 weeks.

In certain aspects, the anti-PD-1 antibody is administered at a flat dose of about 200 mg once about every 3 weeks and the anti-CTLA-4 antibody is administered at a flat dose of about 80 mg once about every 6 weeks. In some aspects, the anti-PD-1 antibody is administered at a flat dose of about 200 mg once about every 2 weeks and the anti-CTLA-4 antibody is administered at a dose of about 80 mg once about every 6 weeks. In some aspects, the anti-PD-1 antibody is administered at a flat dose of about 240 mg once about every 2 weeks and the anti-CTLA-4 antibody is administered at a dose of about 80 mg once about every 6 weeks. In some aspects, the anti-PD-1 antibody is administered at a flat dose of about 480 mg once about every 4 weeks and the anti-CTLA-4 antibody is administered at a dose of about 80 mg once about every 6 weeks.

In certain aspects, the anti-PD-L1 antibody is administered at a dose of about 10 mg/kg once about every 2 weeks and the anti-CTLA-4 antibody is administered at a dose of about 1 mg/kg once about every 6 weeks. In some aspects, the anti-PD-L1 antibody is administered at a dose of about 15 mg/kg once about every 3 weeks and the anti-CTLA-4 antibody is administered at a dose of about 1 mg/kg once about every 6 weeks.

In certain aspects, the anti-PD-L1 antibody is administered at a flat dose of about 800 mg once about every 2 weeks and the anti-CTLA-4 antibody is administered at a dose of about 1 mg/kg once about every 6 weeks. In some aspects, the anti-PD-L1 antibody is administered at a flat dose of about 1200 mg once about every 3 weeks and the anti-CTLA-4 antibody is administered at a dose of about 1 mg/kg once about every 6 weeks.

In certain aspects, the anti-PD-L1 antibody is administered at a flat dose of about 800 mg once about every 2 weeks and the anti-CTLA-4 antibody is administered at a flat dose of about 80 mg once about every 6 weeks. In some aspects, the anti-PD-L1 antibody is administered at a flat dose of about 1200 mg once about every 3 weeks and the anti-CTLA-4 antibody is administered at a dose of about 80 mg once about every 6 weeks.

In some aspects, the anti-PD-1 antibody, e.g., nivolumab, is administered at a dose of about 3 mg/kg and the anti-CTLA-4 antibody is administered at a dose of about 1 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks. In some aspects, the anti-PD-1 antibody, e.g., nivolumab, is administered at a dose of about 1 mg/kg and the anti-CTLA-4 antibody is administered at a dose of about 3 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks.

### II.C.5. Additional Anticancer Therapies

In some aspects of the present disclosure, the methods disclosed herein further comprise administering an anti-PD-1 antibody (or an anti-PD-L1 antibody) and an additional anticancer therapy. In certain aspects, the method comprising administering an anti-PD-1 antibody (or an anti-PD-L1 antibody), an anti-CTLA-4 antibody, and an additional anticancer therapy. The additional anticancer therapy can comprise any therapy known in the art for the treatment of a tumor in a subject and/or any standard-of-care therapy, as disclosed herein. In some aspects, the additional anticancer therapy comprises a surgery, a radiation therapy, a chemotherapy, an immunotherapy, or any combination thereof. In some aspects, the additional anticancer therapy comprises a chemotherapy, including any chemotherapy disclosed herein. In some aspect, the additional anticancer therapy comprises an immunotherapy. In some aspects, the additional anticancer therapy comprises administration of an antibody or antigen-binding portion thereof that specifically binds LAG-3, TIGIT, TIM3, NKG2a, OX40, ICOS, MICA, CD137, KIR, TGFβ, IL-10, IL-8, B7-H4, Fas ligand, CXCR4, mesothelin, CD27, GITR, or any combination thereof.

In some aspects, the additional anticancer agent comprises a therapeutic agent. In some aspects, the therapeutic agent binds a cancer antigen. In some aspects, the therapeutic agent binds a cancer ligand. In some aspects, the additional anticancer agent comprises a cell-based therapy. In some aspects, the additional anticancer agent comprises an immune cell therapy, e.g., a chimeric antigen receptor (CAR) T cell therapy.

### II.D. Tumors

In some aspects, the tumor is derived from a cancer selected from the group consisting of hepatocellular cancer, gastroesophageal cancer, melanoma, bladder cancer, lung cancer, kidney cancer, head and neck cancer, colon cancer, and any combination thereof. In certain aspects, the tumor is derived from a hepatocellular cancer, wherein the tumor has a low stromal signature score. In certain aspects, the tumor is derived from a gastroesophageal cancer, wherein the tumor has a low stromal signature score. In certain aspects, the tumor is derived from a melanoma, wherein the tumor has a low stromal signature score. In certain aspects, the tumor is derived from a bladder cancer, wherein the tumor has a low stromal signature score. In certain aspects, the tumor is derived from a lung cancer, wherein the tumor has a low stromal signature score. In certain aspects, the tumor is derived from a kidney cancer, wherein the tumor has a low stromal signature score. In certain aspects, the tumor is derived from a head and neck cancer, wherein the tumor has a low stromal signature score. In certain aspects, the tumor is derived from a colon cancer, wherein the tumor has a low stromal signature score.

In some aspects, the tumor is an early stage tumor, e.g., eligible for resection. In certain aspects, the subject has received one, two, three, four, five or more prior cancer treatments. In other aspects, the subject is treatment-naïve. In some aspects, the subject has progressed on other cancer treatments. In certain aspects, the prior cancer treatment comprised an immunotherapy. In other aspects, the prior cancer treatment comprised a chemotherapy. In some aspects, the tumor has reoccurred. In some aspects, the tumor is metastatic. In other aspects, the tumor is not metastatic. In some aspects, the tumor is locally advanced.

In some aspects, one or more tumor cells of the tumor express PD-L1, i.e., have surface expression of PD-L1. In some aspects, at least about 2% of tumor cells of the tumor express PD-L1. In some aspects, at least about 5% of tumor cells of the tumor express PD-L1. In some aspects, at least about 10% of tumor cells of the tumor express PD-L1. In some aspects, at least about 15% of tumor cells of the tumor express PD-L1. In some aspects, at least about 20% of tumor cells of the tumor express PD-L1. In some aspects, at least about 25% of tumor cells of the tumor express PD-L1. In some aspects, at least about 30% of tumor cells of the tumor express PD-L1. In some aspects, at least about 35% of tumor cells of the tumor express PD-L1. In some aspects, at least about 40% of tumor cells of the tumor express PD-L1. In some aspects, at least about 45% of tumor cells of the tumor express PD-L1. In some aspects, at least about 50% of tumor cells of the tumor express PD-L1. In some aspects, at least about 55% of tumor cells of the tumor express PD-L1. In some aspects, at least about 60% of tumor cells of the tumor express PD-L1. In some aspects, at least about 65% of tumor cells of the tumor express PD-L1. In some aspects, at least about 70% of tumor cells of the tumor express PD-L1. In some aspects, at least about 75% of tumor cells of the tumor express PD-L1. In some aspects, at least about 80% of tumor cells of the tumor express PD-L1. In some aspects, at least about 85% of tumor cells of the tumor express PD-L1. In some aspects, at least about 90% of tumor cells of the tumor express PD-L1. In some aspects, at least about 95% of tumor cells of the tumor express PD-L1.

In some aspects, the subject has received a prior therapy to treat the tumor and the tumor is relapsed or refractory. In certain aspects, the at least one prior therapy comprises a standard-of-care therapy. In some aspects, the at least one prior therapy comprises a surgery, a radiation therapy, a chemotherapy, an immunotherapy, or any combination thereof. In some aspects, the at least one prior therapy comprises a chemotherapy. In some aspects, the subject has received a prior immuno-oncology (I-O) therapy to treat the tumor and the tumor is relapsed or refractory. In some aspects, the subject has received more than one prior therapy to treat the tumor and the subject is relapsed or refractory. In other aspects, the subject has received either an anti-PD-1 or anti-PD-L1 antibody therapy.

In some aspects, the previous line of therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In one particular aspect, the platinum-based therapy comprises carboplatin.

In some aspects, the at least one prior therapy is selected from a therapy comprising administration of an anticancer agent selected from the group consisting of a platinum agent (e.g., cisplatin, carboplatin), a taxanes agent (e.g., paclitaxel, albumin-bound paclitaxel, docetaxel), vinorelbine, vinblastine, etoposide, pemetrexed, gemcitabine, bevacizumab (AVASTIN®), erlotinib (TARCEVA®), crizotinib (XALKORI®), cetuximab (ERBITUX®), and any combination thereof. In certain aspects, the at least one prior therapy comprises a platinum-based doublet chemotherapy.

In some aspects, the subject has experienced disease progression after the at least one prior therapy. In certain aspects, the subject has received at least two prior therapies, at least three prior therapies, at least four prior therapies, or at least five prior therapies. In certain aspects, the subject has received at least two prior therapies. In one aspect, the subject has experienced disease progression after the at least two prior therapies. In certain aspects, the at least two prior therapies comprises a first prior therapy and a second prior therapy, wherein the subject has experienced disease progression after the first prior therapy and/or the second prior therapy, and wherein the first prior therapy comprises a surgery, a radiation therapy, a chemotherapy, an immunotherapy, or any combination thereof, and wherein the second prior therapy comprises a surgery, a radiation therapy, a chemotherapy, an immunotherapy, or any combination thereof. In some aspects, the first prior therapy comprises a platinum-based doublet chemotherapy, and the second prior therapy comprises a single-agent chemotherapy. In certain aspects, the single-agent chemotherapy comprises docetaxel.

### II.E. Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some aspects, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. Therefore, in some aspects, the pharmaceutical composition for the present disclosure can further comprise recombinant human hyaluronidase enzyme, e.g., rHuPH20.

In some aspects, the method comprises administering an anti-PD-1 antibody (or an anti-PD-L1 antibody) and an anti-CTLA-4 antibody, wherein the anti-PD-1 antibody (or the anti-PD-L1 antibody) is administered in a fixed dose with the anti-CTLA-4 antibody in a single composition. In some aspects, the anti-PD-1 antibody is administered in a fixed dose with the anti-CTLA-4 antibody. In some aspects, the anti-PD-L1 antibody is administered in a fixed dose with the anti-CTLA-4 antibody in a single composition. In some aspects, the ratio of the anti-PD-1 antibody (or the anti-PD-L1 antibody) to the anti-CTLA-4 antibody is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

Treatment is continued as long as clinical benefit is observed or until unacceptable toxicity or disease progression occurs. Nevertheless, in certain aspects, the dosages of the anti-PD-1 antibody, the anti-PD-L1 antibody, and/or the anti-CTLA-4 antibody administered are significantly lower than the approved dosage, i.e., a subtherapeutic dosage, of the agent. The anti-PD-1 antibody, the anti-PD-L1 antibody, and/or the anti-CTLA-4 antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### III. Kits

Also within the scope of the present disclosure are kits comprising (a) an anti-PD-1 antibody or an anti-PD-L1 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from 0.1 to 10 mg/kg body weight of an anti-PD-1 antibody or a dosage ranging from 0.1 to 20 mg/kg body weight of an anti-PD-L1 antibody; and (b) instructions for using the anti-PD-1 antibody or the anti-PD-L1 antibody in the methods disclosed herein. This disclosure further provides a kit for treating a subject afflicted with a tumor, the kit comprising: (a) a dosage ranging from about 4 mg to about 500 mg of an anti-PD-1 antibody or a dosage ranging from about 4 mg to about 2000 mg of an anti-PD-L1 antibody; and (b) instructions for using the anti-PD-1 antibody or the anti-PD-L1 antibody in the methods disclosed herein. In some aspects, this disclosure provides a kit for treating a subject afflicted with a tumor, the kit comprising: (a) a dosage ranging from 200 mg to 800 mg of an anti-PD-1 antibody or a dosage ranging from 200 mg to 1800 mg of an anti-PD-L1 antibody; and (b) instructions for using the anti-PD-1 antibody or the anti-PD-L1 antibody in the methods disclosed herein.

In certain aspects for treating human patients, the kit comprises an anti-human PD-1 antibody disclosed herein, e.g., nivolumab or pembrolizumab. In certain aspects for treating human patients, the kit comprises an anti-human PD-L1 antibody disclosed herein, e.g., atezolizumab, durvalumab, or avelumab.

In some aspects, the kit further comprises an anti-CTLA-4 antibody. In certain aspects for treating human patients, the kit comprises an anti-human CTLA-4 antibody disclosed herein, e.g., ipilimumab, tremelimumab, MK-1308, or AGEN-1884.

In some aspects, the kit further includes a stromal gene panel assay disclosed herein. In some aspects, the kit further includes instructions to administer the anti-PD-1 antibody or the anti-PD-L1 antibody to a subject identified as having a low stromal signature score, according to the methods disclosed herein. In other aspects, the kit further includes an anti-CTLA-4 antibody and instructions to administer (a) the anti-PD-1 antibody or the anti-PD-L1 antibody and (b) the anti-CTLA-4 antibody to a subject identified as having a low stromal signature score, according to the methods disclosed herein.

All of the references cited above, as well as all references cited herein, are incorporated herein by reference in their entireties.

The following examples are offered by way of illustration and not by way of limitation.

### EXAMPLES

### Example 1: Assessment of Biomarkers in Relation to Clinical Outcomes in Nivolumab Plus Chemotherapy-Treated Patients with Advanced Gastric Cancer/Gastroesophageal Junction Cancer/Esophageal Adenocarcinoma

Gastric cancer (GC), including gastroesophageal junction (GEJ) cancer, is the fourth leading cause of cancer-related deaths worldwide with adenocarcinoma being the most common (>90%) histological type of gastric and GEJ cancer. (Janjigian et al., Lancet 398(10294):27-40 (2021); see, GLOBOCAN 2020. Cancer fact sheets, stomach. 2020; and Ajani et al., Nat. Rev. Dis. Primers 3(17036) (2017)). Adenocarcinoma accounts for approximately 65% and 40% of oesophageal cancer in North America and Europe, respectively. (Janjigian et al., Lancet 398(10294):27-40 (2021); see, Arnold et al., Gut 69(9):1564-1571 (2020)). Fluoropyrimidine in combination with platinum-based chemotherapy is the standard first-line treatment for unresectable advanced or metastatic human epidermal growth factor receptor 2 (HER2) negative gastric and GEJ adenocarcinoma and results in poor survival with a median overall survival (OS) of less than 1 year. (Janjigian et al., Lancet 398(10294):27-40 (2021); see, National Comprehensive Cancer Network (NCCN). NCCN Clinical Practice Guidelines in Oncology (NCCN Guidelines®). Gastric Cancer. Version 2.2021. https://www.nccn.org/professionals/physician_gls/pdf/gastric.pdf (accessed Mar. 17, 2021); Smyth et al., Ann. Oncol. 27(suppl 5):v38-v49 (2016); Wang et al., Cancer Commun. 39(1):10 (2019); Japanese Gastric Cancer A. Japanese gastric cancer treatment guidelines 2018 (5th edition), Gastric Cancer 24(1):1-21 (2021); Catenacci et al., Lancet Oncol. 18(11):1467-1482 (2017); Fuchs et al., Lancet Oncol. 20(3):420-435 (2019); Lordick et al., Lancet Oncol. 14(6):490-499 (2013); and Shah et al., JAMA Oncol. 3(5):620-627 (2017)). Gastric/GEJ and esophageal adenocarcinomas (EAC) have similarly poor clinical outcomes with systemic chemotherapy in an advanced setting. (Janjigian et al., Lancet 398(10294):27-40 (2021); see, Pape et al., J. Clin. Oncol. 38(suppl 4):308-308 (2020); and Chau et al., Ann. Oncol. 20(5):885-891 (2009)). Despite several agents having been evaluated as first-line treatment for HER2-negative gastric and GEJ adenocarcinoma, none have been shown to significantly prolong survival relative to chemotherapy. (Janjigian et al., Lancet 398(10294):27-40 (2021); see, Catenacci et al., Lancet Oncol. 18(11):1467-1482 (2017); Fuchs et al., Lancet Oncol. 20(3):420-435 (2019); Lordick et al., Lancet Oncol. 14(6):490-499 (2013); Shah et al., JAMA Oncol. 3(5):620-627 (2017); and Shitara et al., JAMA Oncol. 6(10):1571-1580 (2020)). Nivolumab (“NIVO”) binds to PD-1 receptors, which are expressed primarily on activated T cells, and thus prevents binding of the PD-L1 and PD-L2 ligands, which are expressed on tumor cells. NIVO in combination with chemotherapy demonstrated a superior OS versus chemotherapy in the first-line treatment of advanced or metastatic GC/GEJC/EAC in Clinical Trial NCT02872116. Clinically meaningful progression-free survival (PFS) benefit, a higher rate of durable objective responses, and an acceptable safety profile were also observed. (See, Janjigian et al., Lancet 398(10294):27-40 (2021)). NIVO in combination with chemotherapy is approved in many countries, including the United States, as a first-line treatment in patients with advanced or metastatic GC, GEJC, and EAC based on results from Clinical Trial NCT02872116. (OPDIVO® (nivolumab) [package insert]. Princeton, NJ: Bristol Myers Squibb).

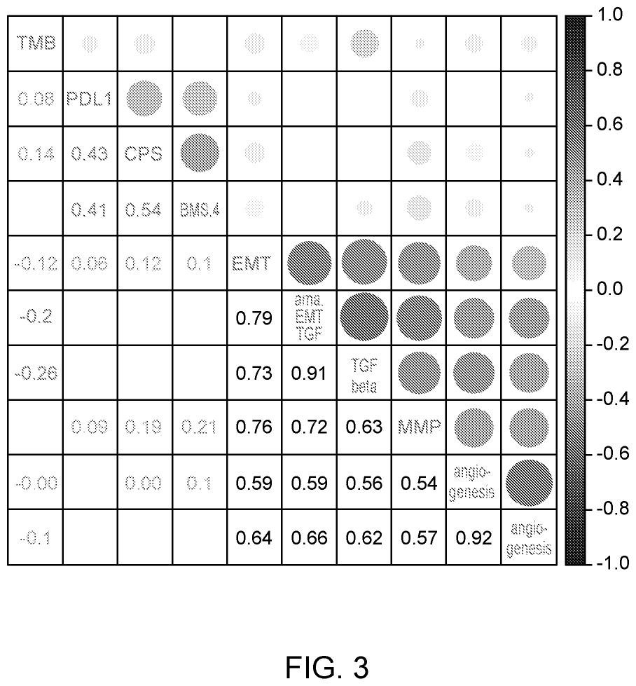

Biomarkers such as tumor mutational burden (TMB) and inflammatory gene expression signatures (GES) may be associated with increased efficacy of PD-1 inhibitors. (See Wang et al., Ann. Oncol. 30(9):1479-1486 (2019); Wu et al., Front. Oncol. 9(1161):1-12 (2019); and Lei et al., Clin. Cancer. Res. 27(14):3926-3935 (2021)). Stroma-related and angiogenesis pathways may also affect response to PD-1 inhibition. (See Wang et al., Nat. Commun. 9(3503):1-12 (2018); and Shiuan et al., Cancers 13(1475):1-16 (2021)). Such biomarkers may be correlated to each other (see FIG. 3). The present example is directed to findings from exploratory biomarker efficacy analyses by baseline TMB and GES status of nivolumab-treated patients with previously untreated advanced GC/EJC/EAC from Clinical Trial NCT02872116, a randomized phase 3 study.

### Study Design

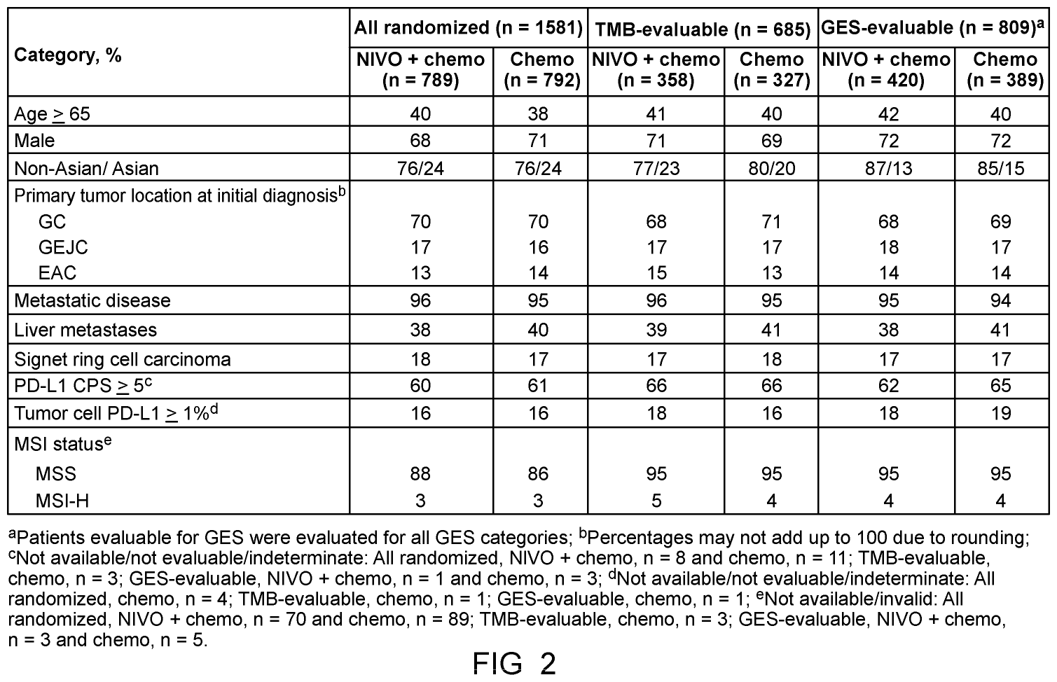

The present data is related to two treatment arms including two groups of subjects of Clinical Trial NCT02872116, which together had a total of 1581 subjects (FIG. 2). The first group comprised 789 subjects and the second group comprised 792 subjects. Baseline characteristics shown in FIG. 2 were balanced between the treatment arms and were consistent between all randomized patients and the TMB- and GES-evaluable populations. Subjects in the first group were administered nivolumab (360 mg every 3 weeks or 240 mg every 2 weeks) plus investigator's choice of chemotherapy (XELOX [capecitabine 1000 mg/m2 twice daily, days 1-14 and oxaliplatin 130 mg/m2, day 1, every 3 weeks] or FOLFOX [leucovorin 400 mg/m2, day 1, fluorouracil 400 mg/m2, day 1 and 1200 mg/m2, days 1-2, and oxaliplatin 85 mg/m2, day 1, every 2 weeks]) and subjects in the second group were administered chemotherapy alone, i.e., investigator's choice of chemotherapy (XELOX [capecitabine 1000 mg/m2 twice daily, days 1-14 and oxaliplatin 130 mg/m2, day 1, every 3 weeks] or FOLFOX [leucovorin 400 mg/m2, day 1, fluorouracil 400 mg/m2, day 1 and 1200 mg/m2, days 1-2, and oxaliplatin 85 mg/m2, day 1, every 2 weeks]). All treatments were administered intravenously except for capecitabine, which was administered orally. Treatment continued until documented disease progression, unacceptable toxicity, withdrawal of consent, or study end. Nivolumab was given for a maximum of 2 years. Chemotherapy was given per local standards. Patients were permitted to continue treatment beyond initial disease progression (per RECIST version 1.1) in the nivolumab plus chemotherapy treatment group, based on investigator judgment.

Dual primary endpoints of Clinical Trial NCT02872116 were OS (time from randomization to death) and PFS (time from randomization to the date of first documented tumor progression or death), evaluated in patients with PD-L1 CPS≥5. Secondary endpoints included OS (PD-L1 CPS≥1, all randomized), OS (PD-L1 CPS≥10), PFS (PD-L1 CPS≥10, ≥1, all randomized), and overall response rate (ORR). Exploratory endpoints included safety, quality of life, and biomarker assessments, which are discussed herein.

Data generated from Clinical Trial NCT02872116 contributed to the USFDA approval of nivolumab in combination with chemotherapy as a first-line treatment in patients with advanced or metastatic GC, GEJC, and EAC.

Eligible patients were 18 years of age or older, with previously untreated, unresectable advanced or metastatic gastric, GEJ, or oesophageal adenocarcinoma, regardless of PD-L1 expression. Other key inclusion criteria were measurable (at least one lesion) or evaluable disease per Response Evaluation Criteria in Solid Tumors (RECIST), version 1.1; Eastern Cooperative Oncology Group performance status of 0 or 1; adequate organ function; and availability to provide a fresh or archival tumor sample to evaluate PD-L1. Patients with prior adjuvant or neoadjuvant chemotherapy, radiotherapy, and/or chemoradiotherapy (administered at least 6 months before randomization) were allowed. Patients with known HER2-positive status; untreated central nervous system metastases peripheral neuropathy (>grade 1); active, known, or suspected autoimmune disease; positive test result for hepatitis B or hepatitis C virus; and known history of positive test for human immunodeficiency virus or known acquired immunodeficiency syndrome were excluded. (Janjigian et al., Lancet 398(10294):27-40 (2021).

Pretreatment tumor samples (fresh or archival if collected within 6 months without intervening systemic therapy) were collected and evaluated for PD-L1 immunohistochemostry (HC) at the time of randomization.

### Exploratory Biomarker Assessments

Samples were analyzed using (i) whole exome sequencing (WES) of baseline tumor tissue and matching blood to assess tissue tumor mutational burden (TMB) and (ii) RNA-sequencing (RNA-seq) of baseline tumor tissue to assess tumor gene expression signatures (GES). Tissue TMB was defined as the total number of somatic missense mutations. TMB-high was defined as ≥199 mutations/exome, and TMB-low as <199 mutations/exome (199 mutations/exome per whole exome sequencing is equivalent to 10 mutations/megabase per Foundation Medicine F1CDx panel). (Chang et al., Mol. Diagn. Ther. 23(4):507-520 (2019)). GES-high, -medium, and -low were defined by signature score tertiles. TMB and GES were assessed for their association with clinical outcomes including overall survival. Association of TMB and GES with OS was evaluated using an unstratified Cox model.

TABLE 16
GES gene lists.

| Category | GES | Gene list |
|---|---|---|
| Inflammation | 4-Gene Inflammatory¹ | CD274, LAG3, CD8A, STAT1 |
| Stroma-related | 51-Gene Stroma/Epithelial- | AEBP1, COL1A2, CRISPLD2, SPARC, COL3A1, COL5A1, |
| | to-Mesenchymal | VCAN, COL15A1, MMP2, PDGFRB, PCOLCE, OLFML2B, |
| | Transition/Transforming | COL6A3, THY1, FSTL1, GPR124, EDNRA, MXRA8, THBS2, |
| | Growth Factor Beta (51- | AXL, COL5A2, NID2, COL8A1, DCN, GGT5, ANGPTL2, |
| | Stroma)² | CD248, LAMA4, GLT8D2, FBN1, ELTD1, CCDC80, CD93, |
| | | RUNX1T1, LRRC32, MSRB3, HEG1, COL6A2, HSPA12B, |
| | | OLFML1, TSHZ3, ANTXR1, FILIP1L, KIAA1462, ADAMTS2, |
| | | ITGA11, WISP1, CDH11, ECM2, FAM26E, PODN |
| | 12-Gene Epithelial-to- | CDH1, CDH2, MMP1, MMP2, ITGA1, ITGA2, ITGA3, ITGA5, |
| | Mesenchymal Transition (12- | ITGA7, ITGA11, TGFB1, TFGBI |
| | EMT) | |
| | 9-Gene Transforming Growth | TGFB1, TGFBR2, ACTA2, COL4A1, TAGLN, SH3PXD2A, |
| | Factor Beta (9-TGF-β) | TWIST1, ZEB1, ZEB2 |
| | 2-Gene Matrix | MMP2, MMP9 |
| | Metallopeptidase (2-MMP) | |
| Angiogenesis | 16-Gene Angiogenesis (16- | VEGFA, CD34, ANGPTL4, KDR, TEK, NDUFA4L2, ANGPT2, |
| | Angio)² | ESM1, CXCR7, SEMA5B, FLT1, TIE1, CDH6, DLL4, FLT4, |
| | | ENPEP |
| | 6-Gene Angiogenesis (6- | VEGFA, KDR, ESM1, PECAM1, ANGPTL4, CD34 |
| | Angio)^{3, 4} | |

¹Lei et al., Clin. Cancer. Res. 27(14): 3926-3935 (2021);
²Cristescu et al., Clin. Cancer. Res. (2022) [Epub ahead of print];
³Brauer et al., Clin. Cancer. Res. 19(13): 3681-3692 (2013);
⁴McDermott et al., Nat. Med. 24(6): 749-757 (2018).

GES included a 4-gene inflammatory (inflam) (Lei et al., Clin. Cancer. Res. 27(14):3926-3935 (2021)), a 12-gene epithelial mesenchymal transition (EMT), 6-gene angiogenesis (angio) (McDermott et al., Nat. Med. 24(6):749-757 (2018)), and other stroma- and angiogenesis-related signatures. (See Table 16).

### Exploratory Biomarker Analysis

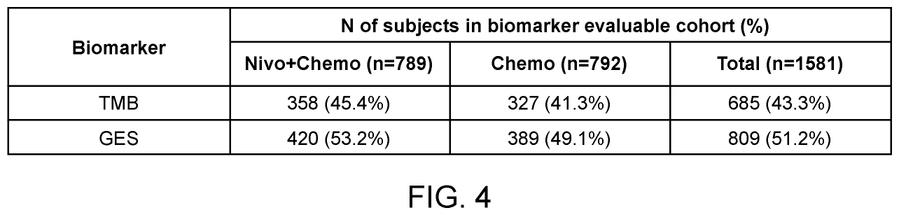

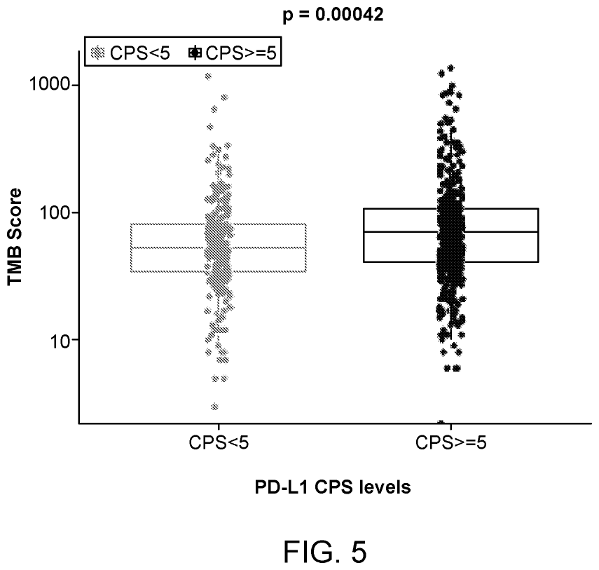

Of 1581 patients randomized to receive NIVO+chemo or chemo, 685 (430%) were evaluable for TMVB (NIVO+chemo 45%, chemo 41%) and 809 (51%) were evaluable for GES (NIVO+chemo 53%, chemo 49%) (FIG. 4). TMB in general was found to be higher in patients with a PD-ligand (L)1l combined positive score (CPS) ≥5 (see FIG. 5). In patients with a PD-L1 CPS ≥5, OS benefit with NIVO+chemo vs. chemo was seen in TMVB-H patients (HR 0.44), which accounted for 10% of the total population, and non-TMB-H patients (HR 0.75) (see Table 17). Similar results were observed when excluding MSI-H patients, which are discussed herein. OS benefit was observed across multiple GES subgroups, although low EMT and angio signature tertiles were associated with greater OS benefit (see Table 17). Similar results were observed with other stroma- and angiogenesis-related GES for all randomized patients and patients with PD-L1 CPS <5, which are discussed herein.

TABLE 17
Overall survival benefit by TMB and GES status.

| | PD-L1 CPS ≥ 5 | | | |
|---|---|---|---|---|
| | | GES | | |
| | TMB | Inflam | EMT | Angio |
| All evaluable, | 451 (100) | 513 (100) | | |
| n (%) | | | | |
| HR | 0.71 | 0.72 | | |
| (95% CI) | (0.58-0.87) | (0.59-0.87) | | |
| High, n (%) | 43 (10) | 171 (33) | 171 (33) | 171 (33) |
| HR | 0.44 | 0.65 | 0.88 | 0.86 |
| (95% CI) | (0.21-0.93) | (0.45-0.93) | (0.64-1.22) | (0.61-1.20) |
| Medium, n (%) | — | 171 (33) | 171 (33) | 171 (33) |
| HR | — | 0.65 | 0.80 | 0.69 |
| (95% CI) | | (0.47-0.91) | (0.57-1.12) | (0.50-0.97) |
| Low, n (%) | 408 (90) | 171 (33) | 171 (33) | 171 (33) |
| HR | 0.75 | 0.77 | 0.57 | 0.62 |
| (95% CI) | (0.61-0.93) | (0.55-1.07) | (0.41-0.80) | (0.44-0.86) |
| | All randomized | | | |
| | | GES | | |
| | TMB | Inflam | EMT | Angio |
| All evaluable, | 685 (100) | 809 (100) | | |
| n (%) | | | | |
| HR | 0.79 | 0.78 | | |
| (95% CI) | (0.67-0.93) | (0.67-0.91) | | |
| High, n (%) | 57 (8) | 270 (33) | 270 (33) | 270 (33) |
| HR | 0.48 | 0.70 | 0.88 | 0.91 |
| (95% CI) | (0.25-0.93) | (0.53-0.93) | (0.68-1.15) | (0.70-1.19) |
| Medium, n (%) | — | 269 (33) | 269 (33) | 269 (33) |
| HR | — | 0.83 | 0.84 | 0.79 |
| (95% CI) | | (0.64-1.08) | (0.65-1.10) | (0.61-1.03) |
| Low, n (%) | 628 (92) | 270 (33) | 270 (33) | 270 (33) |
| HR | 0.83 | 0.75 | 0.68 | 0.66 |
| (95% CI) | (0.70-0.99) | (0.58-0.97) | (0.52-0.88) | (0.51-0.86) |

HR data are NIVO + chemo vs chemo.

### Tumor Mutational Burden

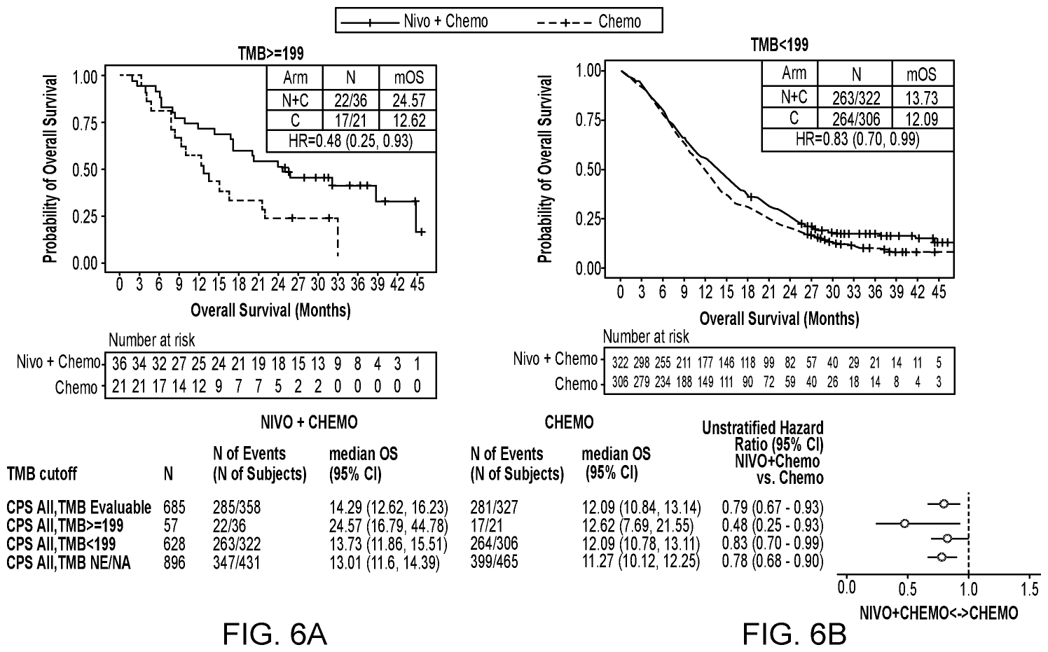

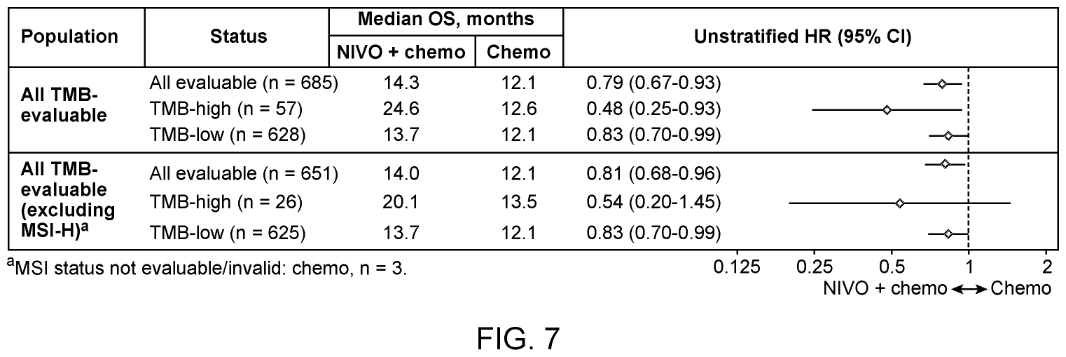

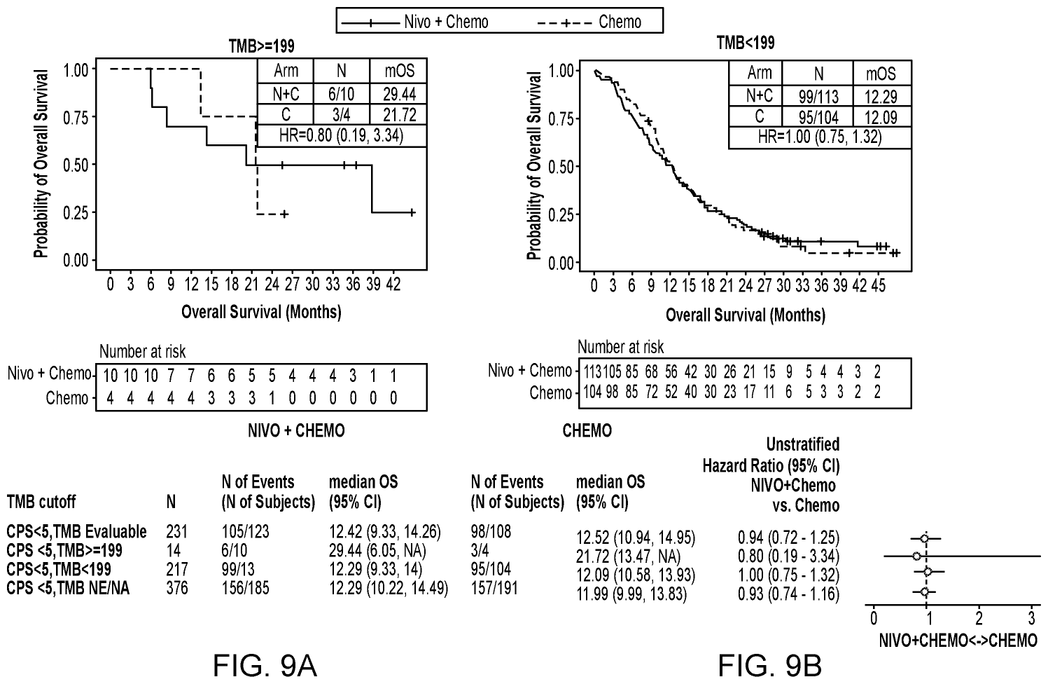

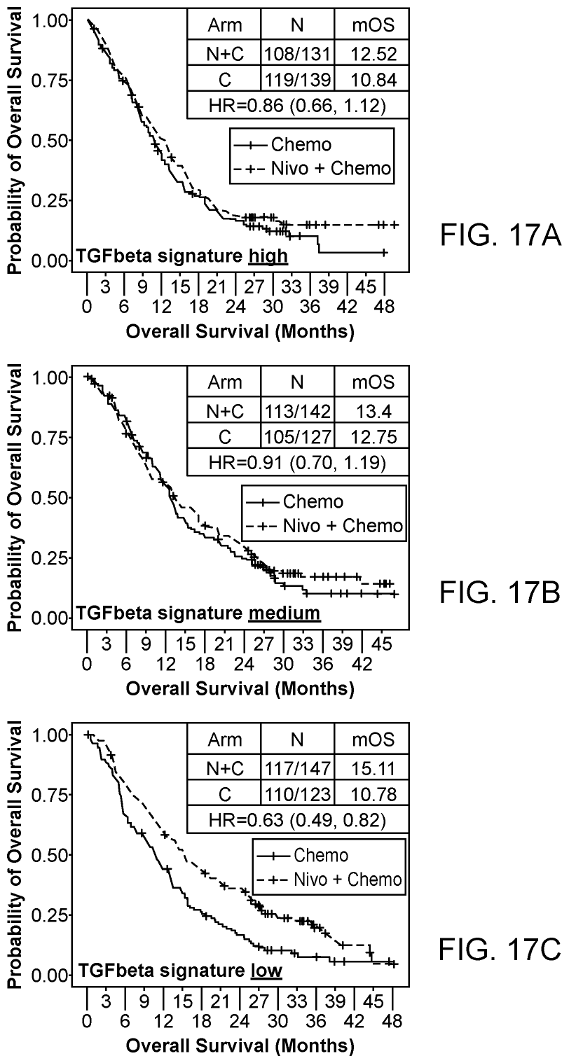

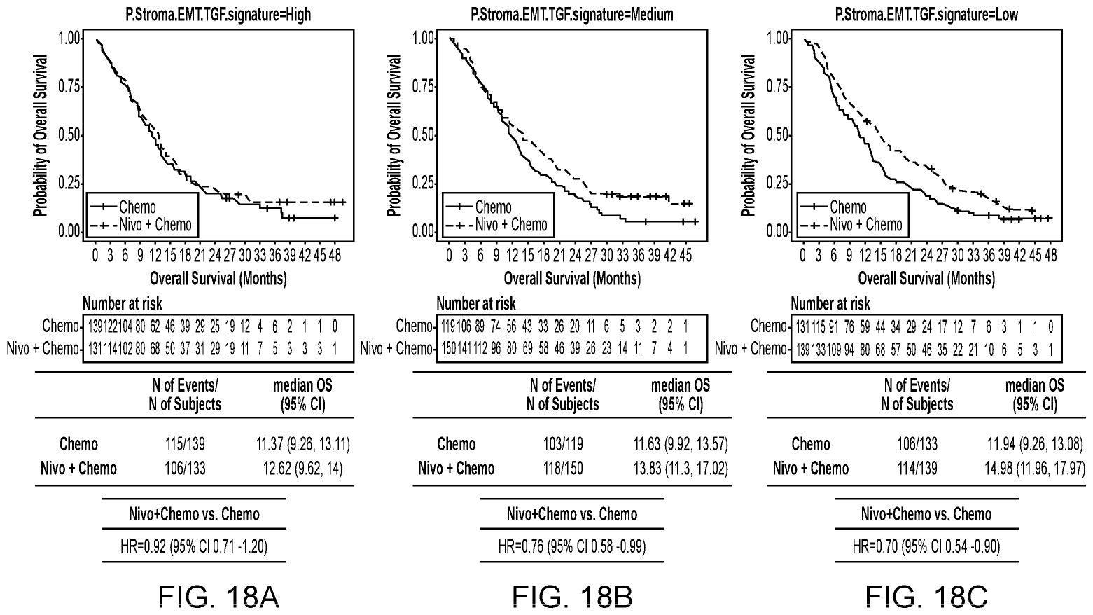

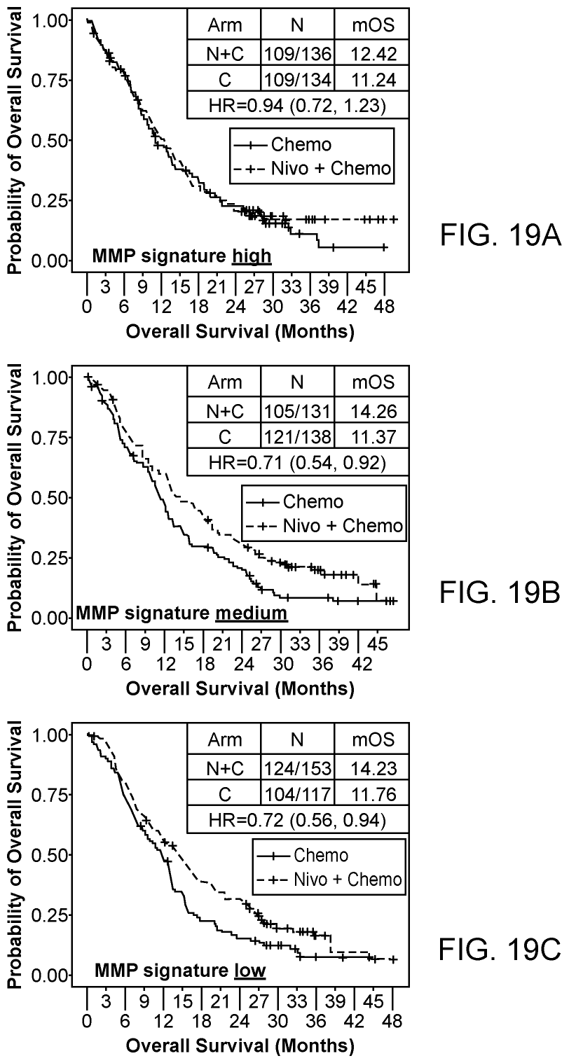

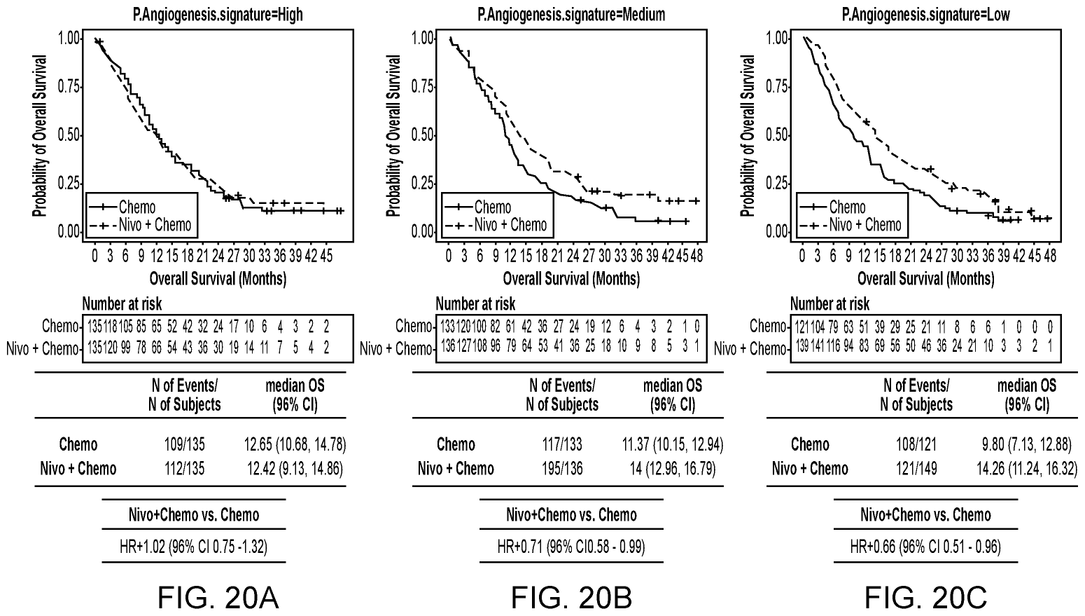

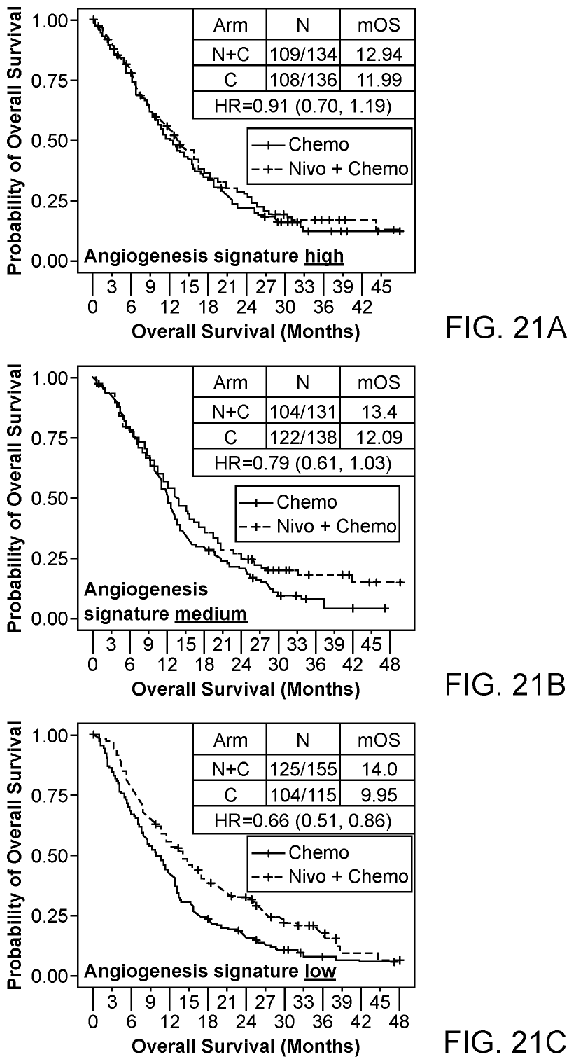

OS benefit with NIVO+chemo vs chemo was observed regardless of TMVB status (see FIGS. 6A-61B). The magnitude of benefit appeared higher in patients with TMB-high tumors, more than half of whom also had MSI-H tumors. Results were consistent when patients with MSI-H tumors were excluded. (See FIG. 7).

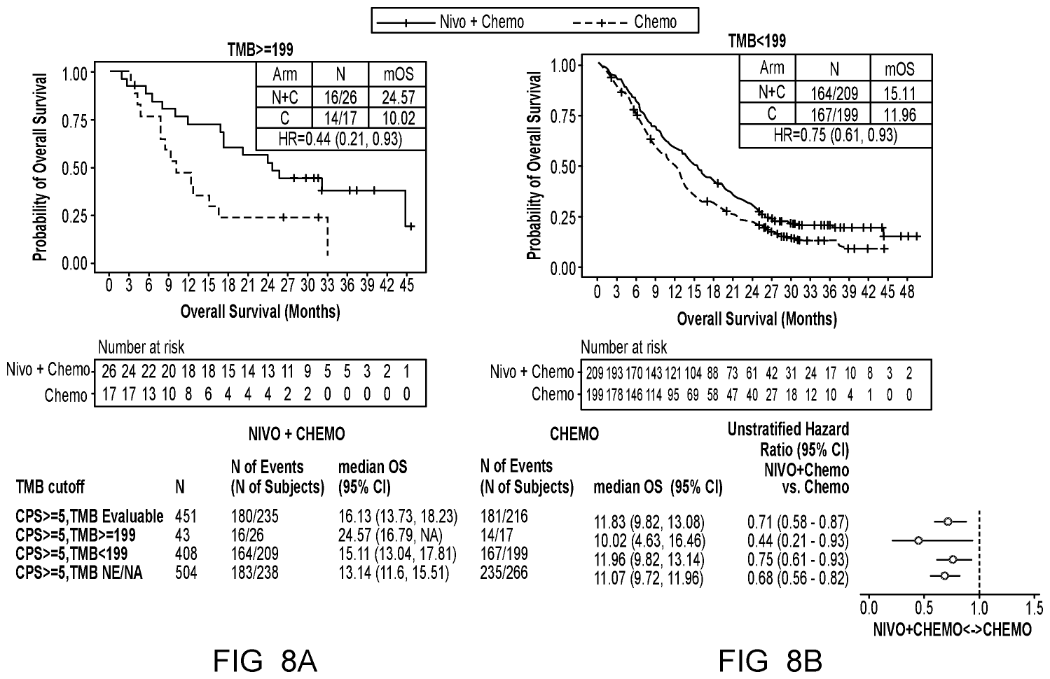

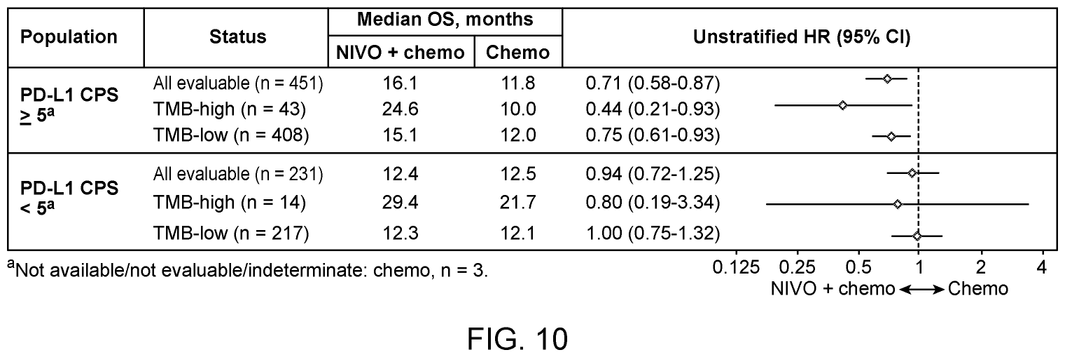

Similar observations of the OS benefit with NIVO+chemo vs chemo were made in the CPS>=5 and CPS<5 subgroups (see FIGS. 8A-81B and 9A-91B). TMB-high status was associated with numerically lower HRs for NIVO+chemo vs chemo in patients with PD-L1 CPS ≥5 and <5, similar to the all TMB-evaluable population (see FIG. 10).

### 4-Gene Inflammatory Gene Expression Signature

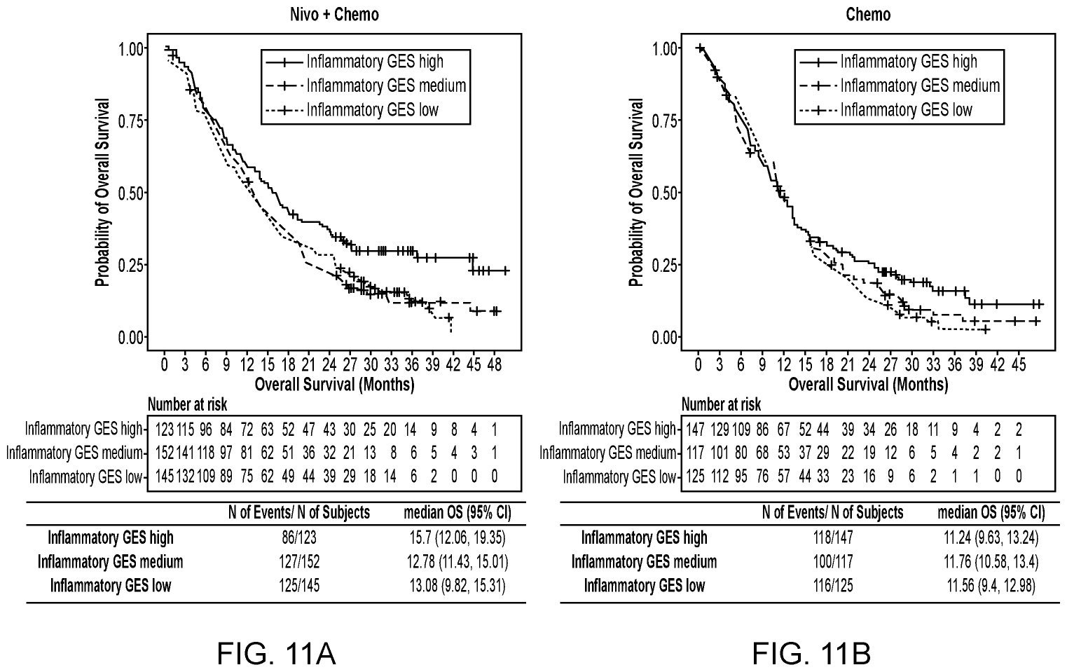

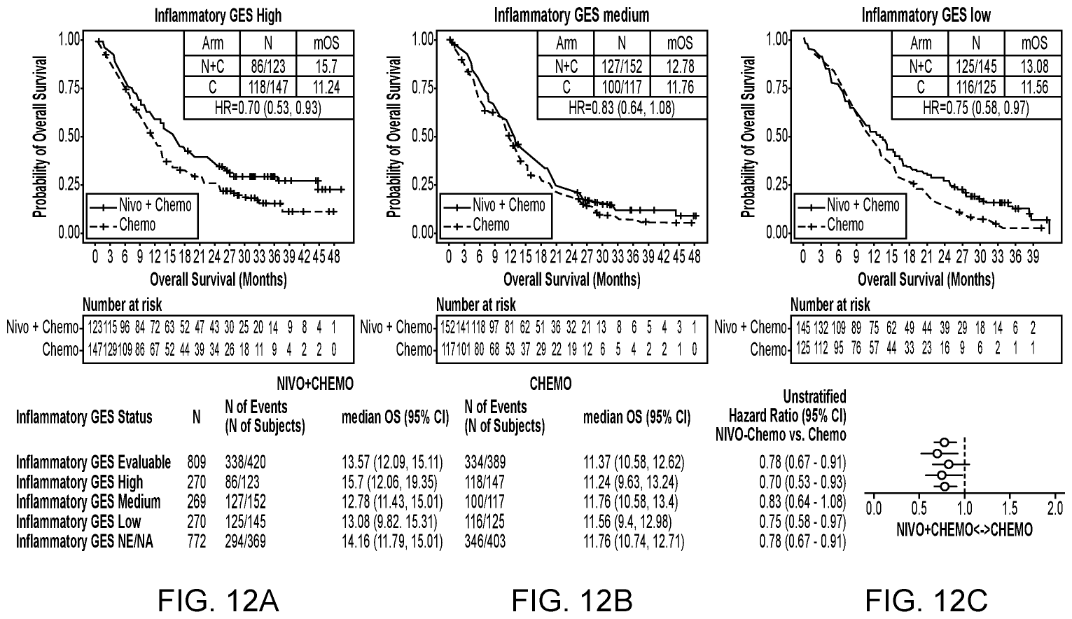

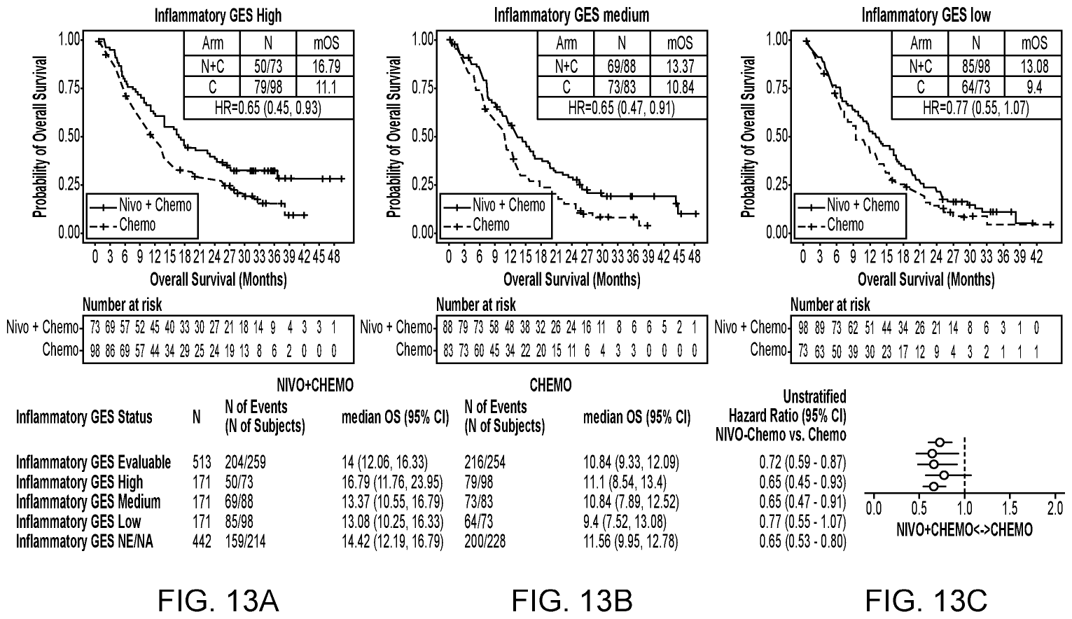

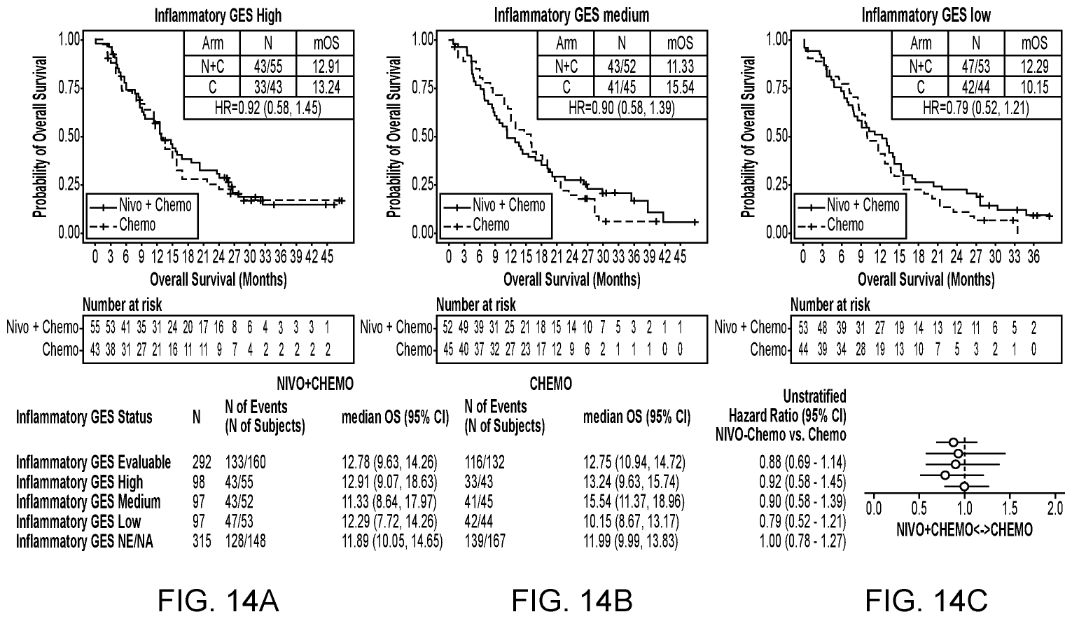

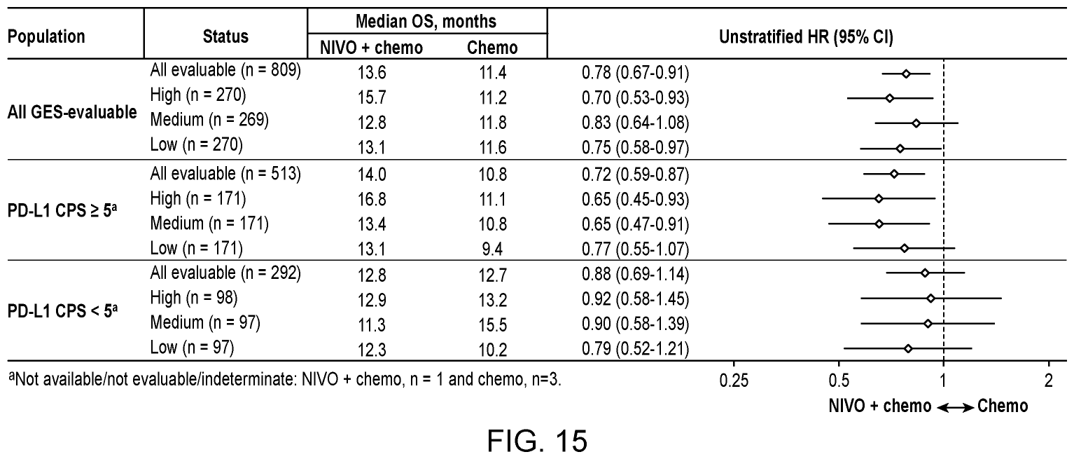

OS benefit with NIVO+chemo vs chemo was observed across 4-gene inflammatory GES subgroups (see FIGS. 11A-11B). No apparent association was observed between 4-gene inflammatory GES status and magnitude of OS benefit (see FIGS. 12A-12C). Results were consistent in patients with PD-L1 CPS ≥5 and <5 (see FIGS. 13A-13C and FIGS. 14A-14C). (See FIG. 15).

### Stroma-Related and Angiogenesis Gene Expression Signatures

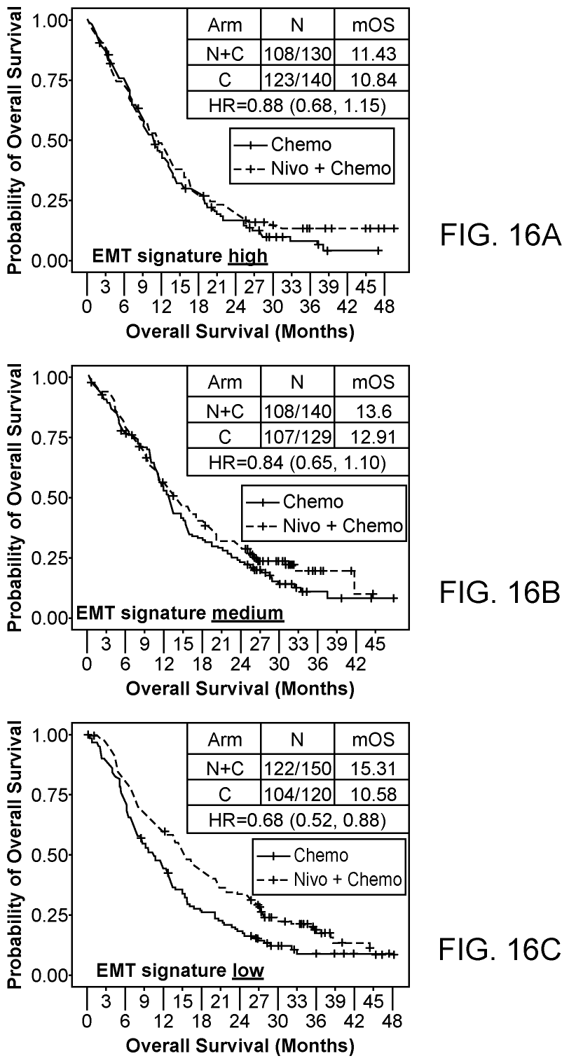

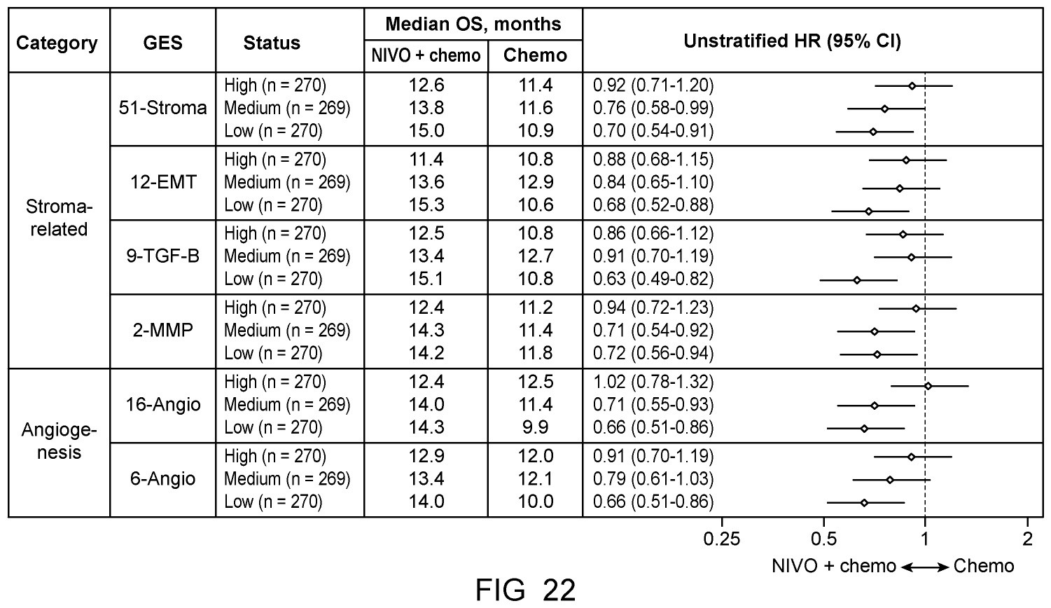

OS favored NIVO+chemo vs chemo across most stroma-related and angiogenesis GES subgroups in GES-evaluable patients. Low levels of stroma-related and angiogenesis gene expression signatures were found to be associated with better OS benefits (see FIGS. 16A-16C, 17A-17C, 18A-18C, 19A-19C, 20A-20C, and 21A-21C). Lower stroma-related and angiogenesis GES appeared to be associated with numerically lower HRs for NIVO+chemo vs chemo. (See FIG. 22).

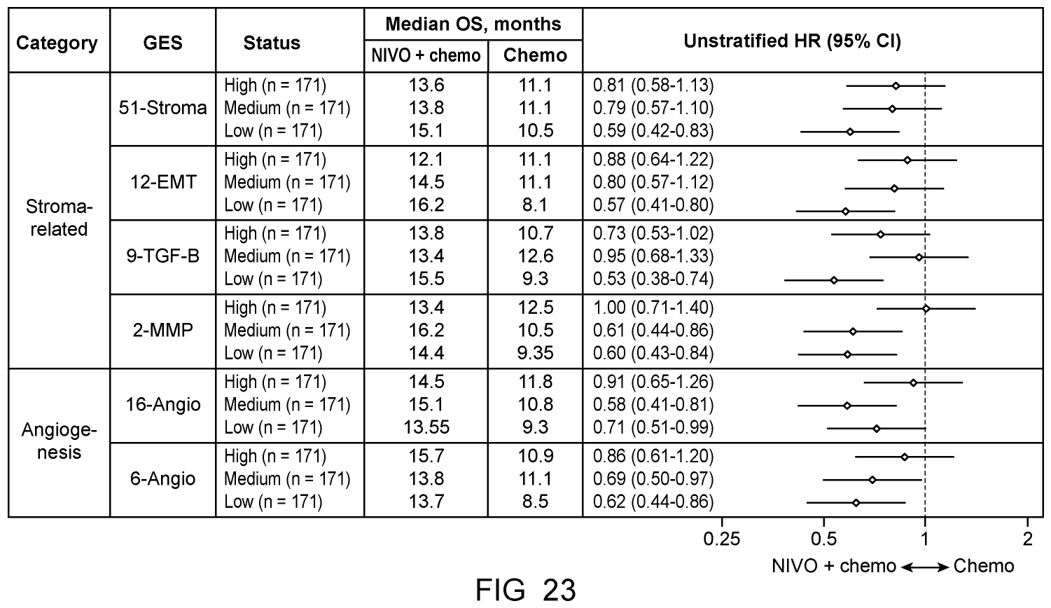

Lower stroma-related and angiogenesis GES appeared to be associated with numerically lower HRs for NIVO+chemo vs chemo in patients with PD-L1 CPS >5, similar to the all GES-evaluable population. (See FIG. 23).

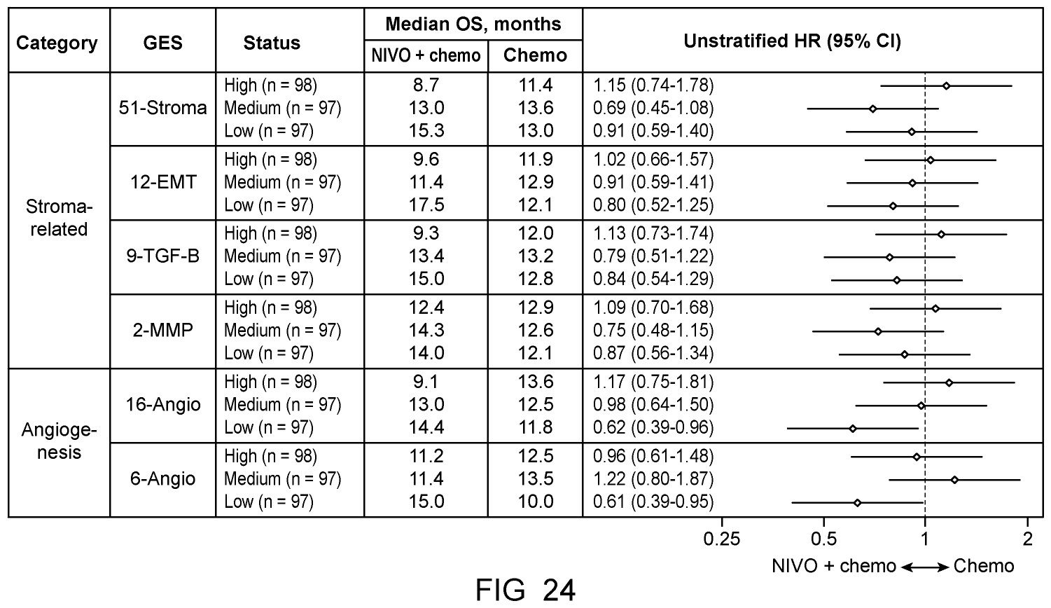

Lower stroma-related and angiogenesis GES appeared to be associated with numerically lower HRs for NIVO+chemo vs chemo in patients with PD-L1 CPS <5, similar to the all GES-evaluable population. (See FIG. 24).

### SUMMARY

In previously untreated advanced gastric cancer/gastroesophageal junction cancer/esophageal adenocarcinoma, a high tumor mutational burden was observed to be associated with clinical benefit and increased survival with immuno-oncology therapy. In addition, a low stromal expression signature and a low angiogenesis expression signature were each observed to be associated with clinical benefit and increased survival with immuno-oncology therapy in previously untreated advanced gastric cancer/gastroesophageal junction cancer/esophageal adenocarcinoma.

### Example 2: Assessment of Biomarkers in Relation to Clinical Outcomes in Nivolumab-Treated Patients with Advanced Esophageal Squamous-Cell Carcinoma

An open-label, phase 3 trial was conducted to evaluate the efficacy and safety of both an immune checkpoint inhibitor in combination with chemotherapy and a dual immune checkpoint inhibitor combination in previously untreated patients with advanced esophageal squamous-cell carcinoma. Adults with previously untreated, unresectable advanced, recurrent, or metastatic esophageal squamous-cell carcinoma were randomly assigned in a 1:1:1 ratio to receive nivolumab plus chemotherapy, nivolumab plus the monoclonal antibody ipilimumab, or chemotherapy. The primary end points were overall survival and progression-free survival, as determined by blinded independent central review. Hierarchical testing was performed first in patients with tumor-cell programmed death ligand 1 (PD-L1) expression of 1% or greater and then in the overall population (all randomly assigned patients).

Eligible patients were at least 18 years of age; had unresectable advanced, recurrent, or metastatic esophageal squamous-cell carcinoma, regardless of PD-L1 expression status; had disease that was not amenable to curative treatments; and had not received previous systemic therapy for advanced disease. Patients had histologically confirmed esophageal squamous-cell or adenosquamous-cell carcinoma and had measurable disease, according to Response Evaluation Criteria in Solid Tumors (RECIST), version 1.1.

Patients were randomly assigned in a 1:1:1 ratio to receive nivolumab (administered intravenously at a dose of 240 mg every 2 weeks) plus chemotherapy (consisting of a 4-week cycle of intravenous fluorouracil at a dose of 800 mg per square meter of body-surface area on days 1 through 5 and intravenous cisplatin at a dose of 80 mg per square meter on day 1); nivolumab (administered intravenously at a dose of 3 mg per kilogram of body weight every 2 weeks) plus ipilimumab (administered intravenously at a dose of 1 mg per kilogram every 6 weeks); or chemotherapy alone. Treatment continued until disease progression, unacceptable toxic effects, withdrawal of consent, or the end of the trial. Patients could receive nivolumab or nivolumab plus ipilimumab for a maximum of 2 years.

The primary end points were overall survival and progression-free survival, as determined by blinded independent central review on the basis of RECIST, version 1.1. The secondary end points included the percentage of patients with an objective response, which was also assessed by blinded independent central review on the basis of RECIST, version 1.1. According to the hierarchical testing procedure, the end points were assessed first in patients with tumor-cell PD-L1 expression of 1% or greater and then in the overall population (i.e., all randomly assigned patients in the trial). Key prespecified exploratory end points were the duration of response (as assessed by blinded independent central review), overall survival in subgroups defined according to tumor-cell PD-L1 expression and PD-L1 combined positive score, patient-reported outcomes, and safety. PD-L1 combined positive score was defined as the number of PD-L1-expressing tumor cells, lymphocytes, and macrophages divided by the total number of viable tumor cells and multiplied by 100.

Adverse events were assessed in all the patients who had received at least one dose of the assigned treatment throughout the treatment and follow-up periods; these events were graded according to the National Cancer Institute Common Terminology Criteria for Adverse Events, version 4.0. Patient-reported outcomes were evaluated with the use of the Functional Assessment of Cancer Therapy-Esophageal (FACT-E) questionnaire, which includes the item, “I am bothered by side effects of treatment” (single GP5 item). The threshold for clinically meaningful change for the FACT-E total score was 9.5 points.

### Results

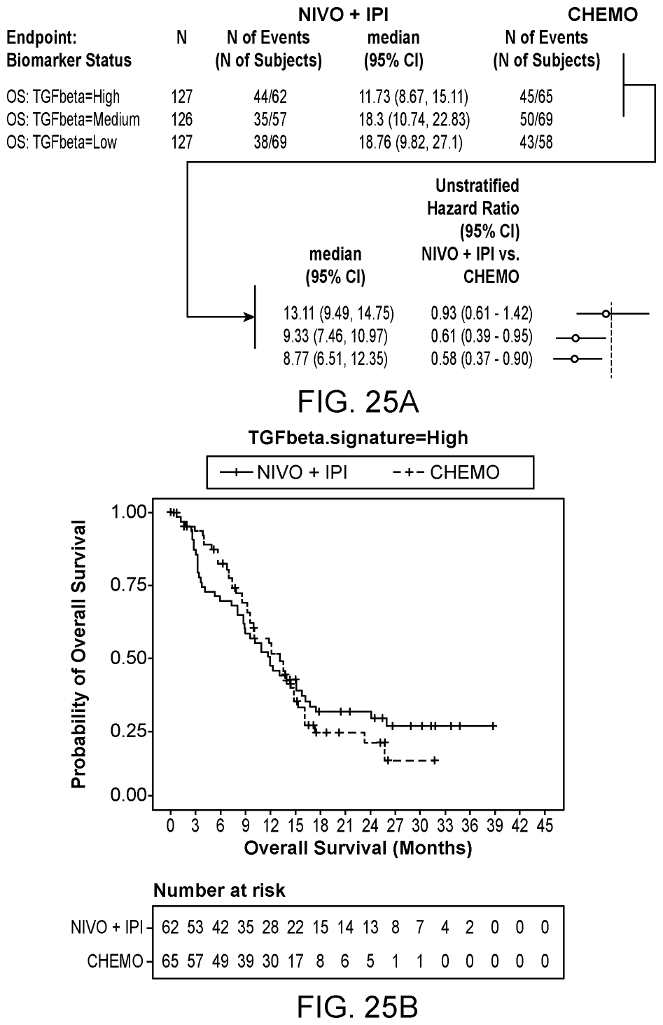

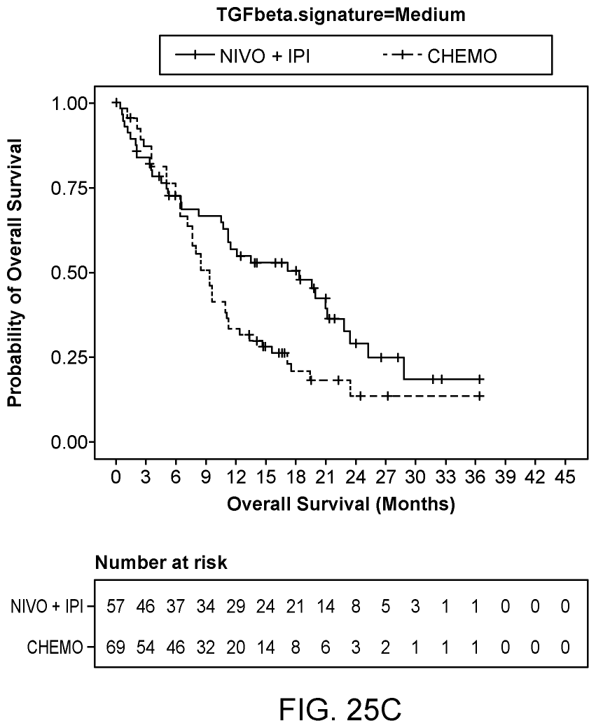

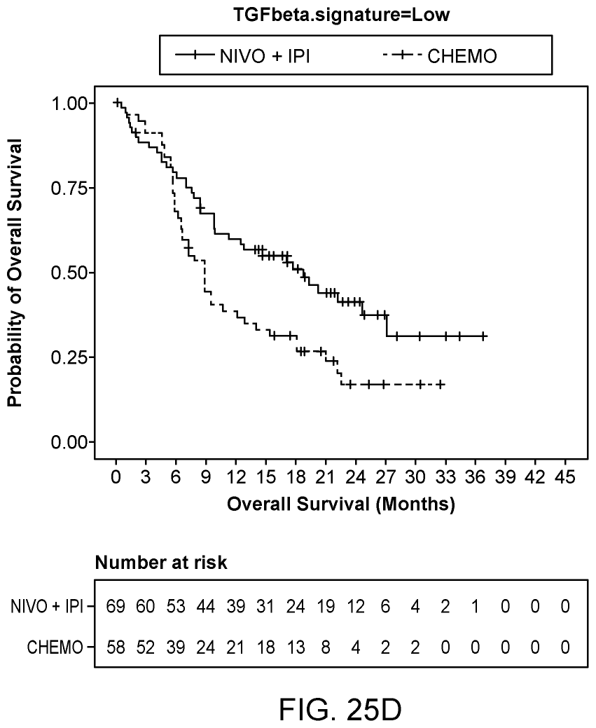

Patients were stratified into three groups based on 9-TGF-β signature, and monitored for probability of overall survival. Patients that exhibited a low 9-TGF-β signature had a higher median disease-free survival (18.76 months) following treatment with nivolumab+ipilimumab as compared to patients treated with chemotherapy (8.77 months) (FIGS. 25A and 25D). Similarly, patients that exhibited a medium 9-TGF-β signature had a similarly high median overall survival (18.3 months) following treatment with nivolumab+ipilimumab as compared to patients treated with chemotherapy (9.33 months) (FIGS. 25A and 25C). Conversely, patients that had a high 9-TGF-β signature exhibited a median overall survival when treated with nivolumab+ipilimumab (11.73 months) that was similar to the levels observed in pateints administered chemotherapy (13.11 months; FIGS. 25A and 25B). These data suggest that patients with a low or medium 9-TGF-β signature are more likely to be responsive to a combination therapy comprising nivolumab and ipilimumab than patients with a high 9-TGF-β signature.

### Example 3: Assessment of Biomarkers in Relation to Clinical Outcomes in Nivolumab-Treated Patients with Resected Esophageal or Gastroesophageal Junction Cancer

A randomized, double-blind, placebo-controlled phase 3 trial was conducted to evaluate a checkpoint inhibitor as adjuvant therapy in patients with esophageal or gastroesophageal junction cancer. Patients were enrolled that had resected esophageal or gastroesophageal junction cancer, and had received neoadjuvant chemoradiotherapy. These patients were enrolled regardless of PD-L1 expression. The inclusion criteria stipulated that at the initial diagnosis, the patients had stage II or III esophageal or gastroesophageal junction cancer and histologically confirmed predominant adenocarcinoma or squamous-cell carcinoma. The patients completed neoadjuvant chemoradiotherapy, followed by complete resection, and were rendered free of disease (defined as no vital tumor present within 1 mm of the proximal, distal, or circumferential resection margins [R0]).

Other key inclusion criteria were residual pathological disease (i.e., the absence of a pathological complete response) with a tumor and node classification of at least ypT1 or ypN1 in the resected specimens (yp denotes the pathological stage after neoadjuvant therapy), an ECOG performance-status score of 0 or 1 (scores range from 0 to 5, with higher scores indicating greater disability), and a complete resection performed within 4 to 16 weeks before randomization.

After neoadjuvant chemoradiotherapy and surgery and within 4 to 16 weeks after surgery, patients were randomly assigned in a 2:1 ratio to receive either nivolumab (administered intravenously at a dose of 240 mg over 30 minutes every 2 weeks for 16 weeks, followed by 480 mg over 30 minutes every 4 weeks beginning at week 17), or placebo (according to the same schedule). Randomization was stratified according to tumor-cell PD-L1 expression (≥1% or <1%, indeterminate, or could not be evaluated), pathological lymph-node status (≥ypN1 or ypN0), and histologic type (squamous-cell carcinoma or adenocarcinoma). The use of nivolumab or placebo continued until disease recurrence, unacceptable toxic effects, or withdrawal of consent occurred. The maximum duration of the trial intervention period was 1 year. Dose modifications were not permitted, but nivolumab or placebo could be interrupted or delayed for a maximum of 6 weeks during the first 16 weeks or for a maximum of 10 weeks during the remainder of the trial intervention period.

The primary end point was disease-free survival (the time from the date of randomization to the first date of disease recurrence or death, whichever occurred first, before subsequent anticancer therapy). Recurrence was defined as the appearance of one or more new lesions (local, regional, or distant in location from the primary resected site, confirmed by imaging or by cytologic or pathological evaluation) as assessed by the investigators. The secondary end points were overall survival and survival at 1, 2, and 3 years. Exploratory end points included safety, distant metastasis-free survival, and patient-reported outcomes (evaluated with the Functional Assessment of Cancer Therapy-Esophageal [FACT-E]scale and the three-level version of the European Quality of Life-5 Dimensions questionnaire [EQ-5D-3L]).

Disease recurrence was evaluated with the use of contrast-enhanced computed tomography (CT) or magnetic resonance imaging at baseline and every 12 weeks from the first date of administration of nivolumab or placebo (±7 days) in the first year, every 12 weeks (±14 days) in the second year, and according to local standards (a minimum of one imaging assessment every 6 to 12 months) between years 3 and 5 (until distant recurrence). If a new lesion was equivocal or unclear, either because of the lesion size or an ambiguous cause, the suspected lesion was confirmed by means of cytologic or histopathologic assessment or by a follow-up imaging evaluation within 4 weeks (if biopsy was not possible). If cytologic or histopathologic assessment or repeat imaging confirmed recurrence, then recurrence was recorded according to the date of the initial imaging. In cases of clinically clear recurrence, the diagnosis could be made on the basis of imaging alone. Lymph-node metastasis was determined by means of CT on the basis of a lymph-node diameter of at least 1 cm in the short axis.

Tumor-cell PD-L1 expression, defined as the percentage of viable tumor cells with partial or complete membrane staining in at least 100 viable tumor cells, was evaluated at two central laboratories with the use of the PD-L1 TIC 28-8 pharmDX assay with the Dako Autostainer Link 48 system (Dako, Agilent Technologies), according to the manufacturer's instructions. A combined positive score was generated as part of a post hoc exploratory analysis by using a formula to rescore the PD-L1-stained slides. The combined positive score was defined as the number of PD-L1-positive tumor cells (with partial or complete membrane staining), lymphocytes, and macrophages (with membrane staining, intracellular staining, or both) divided by the total number of viable tumor cells and multiplied by 100.

### Results

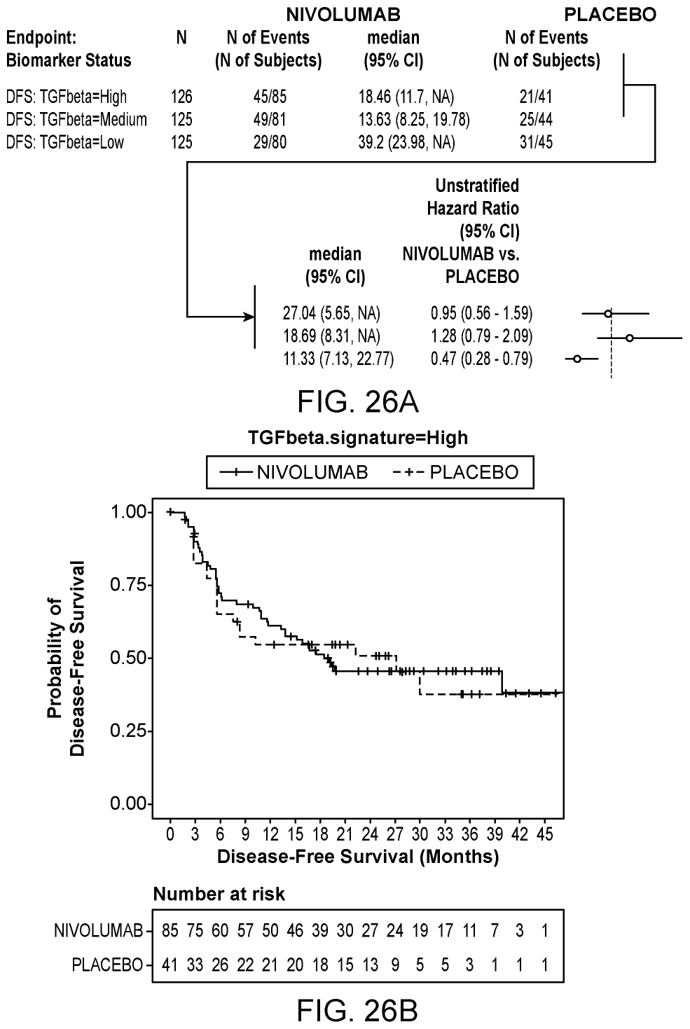

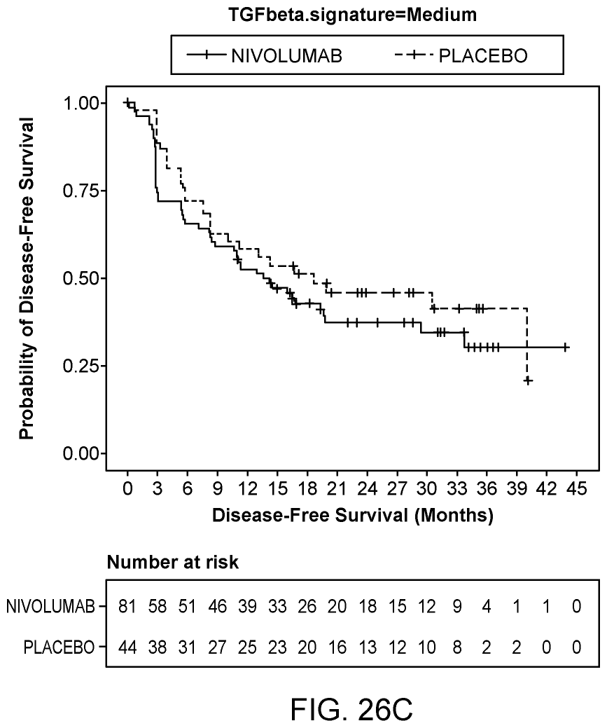

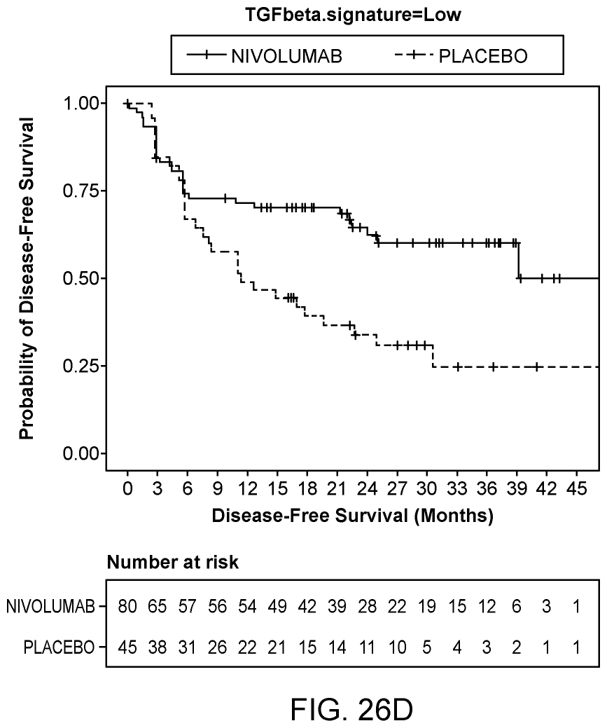

Patients were stratified into three groups based on 9-TGF-β signature, and monitored for probability of disease-free survival. Patients that exhibited a low 9-TGF-β signature had median disease-free survival (39.2 months) following treatment with nivolumab as compared to patients treated with the placebo (11.33 months) (FIGS. 26A and 26D). Conversely, patients that had a high (FIGS. 26A and 26B) or medium (FIGS. 26A and 26C) 9-TGF-β signature exhibited a median disease-free survival when treated with nivolumab that was similar to the levels observed in pateints administered the placebo. These data suggest that patients with a low 9-TGF-β signature are more likely to be responsive to nivolumab therapy than patients with a high 9-TGF-β signature.

1. (canceled)

2. A method for treating a human subject afflicted with a tumor comprising administering (i) an anti-PD-1 antibody or an antigen-binding portion thereof or (ii) an anti-PD-L1 antibody or an antigen-binding portion thereof to the subject, wherein the subject is identified as exhibiting a low stromal signature score prior to the administration;
wherein the stromal signature score is determined by measuring the expression of a panel of stroma-related genes (“stromal gene panel”) in a tumor sample obtained from the subject; and wherein the stromal gene panel comprises at least four genes selected from CDH1, CDH2, MMP1, MMP2, ITGA1, ITGA2, ITGA3, ITGA5, ITGA7, ITGA11, TGFB1, and TGFB1.

3. (canceled)

4. The method of claim 2, wherein the stromal gene panel
(i) comprises CDH1, CDH2, AIP1, MMP2, ITGA1, ITGA2, ITGA3, ITGA5, ITGA7, ITGA11, TGFB1, and TGFB1;
(ii) consists essentially of CDH1, CDH2, MMP1, MMP2, ITGA1, ITGA2, ITGA3, ITGA5, ITGA7, ITGA11, TGFB1, and TGFB1; or
(iii) consists essentially of (i) CDH1, CDH2, MMP1, MMP2, ITGA1, ITGA2, ITGA3, ITGA5, ITGA7, ITGA11, TGFB1, and TGFB1; and (ii) 1 additional stromal gene, 2 additional stromal genes, 3 additional stromal genes, 4 additional stromal genes, 5 additional stromal genes, 6 additional stromal genes, 7 additional stromal genes, 8 additional stromal genes, 9 additional stromal genes, 10 additional stromal genes, 11 additional stromal genes, 12 additional stromal genes, 13 additional stromal genes, 14 additional stromal genes, or 15 additional stromal genes

5. (canceled)

6. The method of claim 4 wherein the additional stromal gene comprises TGFBR2, ACTA2, COL4A1, TAGLN, SH3PXD2A, TWIST1, ZEB1, ZEB2, MMP9, or any combination thereof.

7. (canceled)

8. The method of claim 2, wherein the stromal gene panel consists of CDH1, CDH2, MMP1, MMP2, ITGA1, ITGA2, ITGA3, ITGA5, ITGA7, ITGA11, TGFB1, and TGFB1.

9. (canceled)

10. A method for treating a human subject afflicted with a tumor comprising administering (i) an anti-PD-1 antibody or an antigen-binding portion thereof or (ii) an anti-PD-L1 antibody or an antigen-binding portion thereof to the subject, wherein the subject is identified as exhibiting a low stromal signature score prior to the administration;
wherein the stromal signature score is determined by measuring the expression of a panel of stroma-related genes (“stromal gene panel”) in a tumor sample obtained from the subject; and wherein the stromal gene panel comprises at least four genes selected from TGFB1, TGFBR2, ACTA2, COL4A1, TAGLN, SH3PXD2A, TWIST1, ZEB1, and ZEB2.

11-17. (canceled)

18. A method for treating a human subject afflicted with a tumor comprising administering (i) an anti-PD-1 antibody or an antigen-binding portion thereof or (ii) an anti-PD-L1 antibody or an antigen-binding portion thereof to the subject, wherein the subject is identified as exhibiting a low stromal signature score prior to the administration;
wherein the stromal signature score is determined by measuring the expression of a panel of stroma-related genes (“stromal gene panel”) in a tumor sample obtained from the subject; and wherein the stromal gene panel comprises MMP2 and MIP9.

19-23. (canceled)

24. The method of claim 2, wherein the stromal gene panel consists of less than about 20 stromal genes.

25. The method of claim 2, wherein the low stromal signature score is characterized by a stromal signature score that is lower than an average stromal signature score, wherein the average stromal signature score is determined by averaging or computationally deriving from the stroma signature scores in tumor samples obtained from a population of subjects afflicted with the tumor.

26. The method of claim 25, wherein the average stromal signature score is determined by averaging or computationally deriving from the stroma signature scores in tumor samples obtained from the population of subjects.

27. (canceled)

28. The method of claim 25, wherein the low stromal signature score is characterized by a stromal signature score that is less than about 75% that of the average stromal signature score.

29. (canceled)

30. The method of claim 2, wherein the tumor sample comprises a tumor tissue biopsy.

31. The method of claim 2, wherein the tumor sample comprises a formalin-fixed, paraffin-embedded tumor tissue or a fresh-frozen tumor tissue.

32. The method of claim 2, wherein the expression of the stromal genes in the stromal gene panel is determined by detecting the presence of stromal gene mRNA, the presence of a protein encoded by the stromal gene, or both.

33-39. (canceled)

40. The method of claim 2, wherein the anti-PD-1 antibody or the antigen-binding portion thereof comprises nivolumab, pembrolizumab, or an antigen-binding portion thereof.

41-42. (canceled)

43. The method of claim 2, wherein the anti-PD-1 antibody or the antigen-binding portion thereof is administered (i) at a dose of about 3 mg/kg body weight once about every 2 weeks, (ii) at a flat dose of about 240 mg once about every two weeks, or (iii) at a flat dose of about 480 mg once about every four weeks.

44-66. (canceled)

67. The method ofany one of claim 2, further comprising administering an antibody or an antigen binding portion thereof that binds specifically to cytotoxic T-lymphocyte-associated protein 4 (CTLA-4) (“an anti-CTLA-4 antibody”).

68-69. (canceled)

70. The method of claim 67, wherein the anti-CTLA-4 antibody or the antigen-binding portion thereof comprises ipilimumab, tremelimumab, or an antigen-binding portion thereof.

71-72. (canceled)

73. The method of claim 67, wherein the anti-CTLA-4 antibody or the antigen-binding portion thereof is administered at a dose of 1 mg/kg body weight once every 6 weeks or once about every 4 weeks.

74-77. (canceled)

78. The method of claim 2, wherein the tumor is derived from a hepatocellular cancer, gastroesophageal cancer, melanoma, bladder cancer, lung cancer, kidney cancer, head and neck cancer, colon cancer, rectal cancer, or any combination thereof.

79-96. (canceled)

97. The method of claim 2, further comprising measuring the TMB status of a biological sample obtained from the subject prior to the administering.

98-119. (canceled)
